SEQUENCE LISTING (18 sequences)

SEQ ID NO: 1 - protein, 918 aa, Homo sapiens
MEHGTLLAQPGLWTRDTSWALLYFLCYILPQTAPQVLRIGGIFETVENEPVNVEELAFKFAVTSINRNRTLMPNTTLTYDIQRINLFDSFEASRRACDQLALGVAALFGPSHSSSVSAVQSICNALEVPHIQTRWKHPSVDNKDLFYINLYPDYAAISRAILDLVLYYNWKTVTVVYEDSTGLIRLQELIKAPSRYNIKIKIRQLPSGNKDAKPLLKEMKKGKEFYVIFDCSHETAAEILKQILFMGMMTEYYHYFFTTLDLFALDLELYRYSGVNMTGFRLLNIDNPHVSSIIEKWSMERLQAPPRPETGLLDGMMTTEAALMYDAVYMVAIASHRASQLTVSSLQCHRHKPWRLGPRFMNLIKEARWDGLTGHITFNKTNGLRKDFDLDIISLKEEGTEKAAGEVSKHLYKVWKKIGIWNSNSGLNMTDSNKDKSSNITDSLANRTLIVTTILEEPYVMYRKSDKPLYGNDRFEGYCLDLLKELSNILGFIYDVKLVPDGKYGAQNDKGEWNGMVKELIDHRADLAVAPLTITYVREKVIDFSKPFMTLGISILYRKPNGTNPGVFSFLNPLSPDIWMYVLLACLGVSCVLFVIARFTPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVRDGSTMTFFKKSKISTYEKMWAFMSSRQQTALVRNSDEGIQRVLTTDYALLMESTSIEYVTQRNCNLTQIGGLIDSKGYGVGTPIGSPYRDKITIAILQLQEEGKLHMMKEKWWRGNGCPEEDNKEASALGVENIGGIFIVLAAGLVLSVFVAIGEFIYKSRKNNDIEQAFCFFYGLQCKQTHPTNSTSGTTLSTDLECGKLIREERGIRKQSSVHTV

SEQ ID NO: 2 - protein, 908 aa, Homo sapiens
MKIIFPILSNPVFRRTVKLLLCLLWIGYSQGTTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSPDIWMYILLAYLGVSCVLFVIARFSPYEWYNPHPCNPDSDVVENNFTLLNSFWFGVGALMQQGSELMPKALSTRIVGGIWWFFTLIIISSYTANLAAFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQNIGGIFIVLAAGLVLSVFVAVGEFLYKSKKNAQLEKRSFCSAMVEELRMSLKCQRRLKHKPQAPVIVKTEEVINMHTFNDRRLPGKETMA

SEQ ID NO: 3 - protein, 919 aa, Homo sapiens [sequence omitted]

SEQ ID NO: 4 - protein, 956 aa, Homo sapiens
MPRVSAPLVLLPAWLVMVACSPHSLRIAAILDDPMECSRGERLSITLAKNRINRAPERLGKAKVEVDIFELLRDSEYETAETMCQILPKGVVAVLGPSSSPASSSIISNICGEKEVPHFKVAPEEFVKFQFQRFTTLNLHPSNTDISVAVAGILNFFNCTTACLICAKAECLLNLEKLLRQFLISKDTLSVRMLDDTRDPTPLLKEIRDDKTATIIIHANASMSHTILLKAAELGMVSAYYTYIFTNLEFSLQRMDSLVDDRVNILGFSIFNQSHAFFQEFAQSLNQSWQENCDHVPFTGPALSSALLFDAVYAVVTAVQELNRSQEIGVKPLSCGSAQIWQHGTSLMNYLRMVELEGLTGHIEFNSKGQRSNYALKILQFTRNGFRQIGQWHVAEGLSMDSHLYASNISDTLFNTTLVVTTILENPYLMLKGNHQEMEGNDRYEGFCVDMLKELAEILRFNYKIRLVGDGVYGVPEANGTWTGMVGELIARKADLAVAGLTITAEREKVIDFSKPFMTLGISILYRVHMGRKPGYFSFLDPFSPGVWLFMLLAYLAVSCVLFLVARLTPYEWYSPHPCAQGRCNLLVNQYSLGNSLWFPVGGFMQQGSTIAPRALSTRCVSGVWWAFTLIIISSYTANLAAFLTVQRMDVPIESVDDLADQTAIEYGTIHGGSSMTFFQNSRYQTYQRMWNYMYSKQPSVFVKSTEEGIARVLNSNYAFLLESTMNEYYRQRNCNLTQIGGLLDTKGYGIGMPVGSVFRDEFDLAILQLQENNRLEILKRKWWEGGKCPKEEDHRAKGLGMENIGGIFVVLICGLIVAIFMAMLEFLWTLRHSEATEVSVCQEMVTELRSIILCQDSIHPRRRRAAVPPPRPPIPEERRPRGTATLSNGKLCGAGEPDQLAQRLAQEAALVARGCTHIRVCPECRRFQGLRARPSPARSEESLEWEKTTNSSEPE

SEQ ID NO: 5 - protein, 980 aa, Homo sapiens
MPAELLLLLIVAFASPSCQVLSSLRMAAILDDQTVCGRGERLALALAREQINGIIEVPAKARVEVDIFELQRDSQYETTDTMCQILPKGVVSVLGPSSSPASASTVSHICGEKEIPHIKVGPEETPRLQYLRFASVSLYPSNEDVSLAVSRILKSFNYPSASLICAKAECLLRLEELVRGFLISKETLSVRMLDDSRDPTPLLKEIRDDKVSTIIIDANASISHLILRKASELGMTSAFYKYILTTMDFPILHLDGIVEDSSNILGFSMFNTSHPFYPEFVRSLNMSWRENCEASTYLGPALSAALMFDAVHVVVSAVRELNRSQEIGVKPLACTSANIWPHGTSLMNYLRMVEYDGLTGRVEFNSKGQRTNYTLRILEKSRQGHREIGVWYSNRTLAMNATTLDINLSQTLANKTLVVTTILENPYVMRRPNFQALSGNERFEGFCVDMLRELAELLRFRYRLRLVEDGLYGAPEPNGSWTGMVGELINRKADLAVAAFTITAEREKVIDFSKPFMTLGISILYRVHMGRKPGYFSFLDPFSPAVWLFMLLAYLAVSCVLFLAARLSPYEWYNPHPCLRARPHILENQYTLGNSLWFPVGGFMQQGSEIMPRALSTRCVSGVWWAFTLIIISSYTANLAAFLTVQRMEVPVESADDLADQTNIEYGTIHAGSTMTFFQNSRYQTYQRMWNYMQSKQPSVFVKSTEEGIARVLNSRYAFLLESTMNEYHRRLNCNLTQIGGLLDTKGYGIGMPLGSPFRDEITLAILQLQENNRLEILKRKWWEGGRCPKEEDHRAKGLGMENIGGIFIVLICGLIIAVFVAVMEFIWSTRRSAESEEVSVCQEMLQELRHAVSCRKTSRSRRRRRPGGPSRALLSLRAVREMRLSNGKLYSAGAGGDAGSAHGGPQRLLDDPGPPSGARPAAPTPCTHVRVCQECRRIQALRASGAGAPPRGLGVPAEATSPPRPRPGPAGPRELAEHE

SEQ ID NO: 6 - protein, 530 aa, synthetic construct
  source 1..530: note = extracellular domain from Gluk2
TTHVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSP

SEQ ID NO: 7 - protein, 163 aa, synthetic construct
  source 1..163: note = extracellular domain from Gluk2
AFLTVERMESPIDSADDLAKQTKIEYGAVEDGATMTFFKKSKISTYDKMWAFMSSRRQSVLVKSNEEGIQRVLTSDYAFLMESTTIEFVTQRNCNLTQIGGLIDSKGYGVGTPMGSPYRDKITIAILQLQEEGKLHMMKEKWWRGNGCPEEESKEASALGVQN

SEQ ID NO: 8 - protein, 13 aa, synthetic construct
  source 1..13: note = peptide identified by mass spec
MESPIDSADDLAK

SEQ ID NO: 9 - protein, 938 aa, synthetic construct
  source 1..938: note = SEQ ID NO: 7 from EP3505935, corresponding to NR1 subunit
MSTMHLLTFALLFSCSFARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYNWNHIILLVSDDHEGRAAQKRLETLLEERESKAEKVLQFDPGTKNVTALLMEARELEARVIILSASEDDAATVYRAAAMLNMTGSGYVWLVGEREISGNALRYAPDGIIGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIVTIHQEPFVYVKPTMSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKKEIPRSTLDSFMQPFQSTLWLLVGLSVHVVAVMLYLLDRFSPFGRFKVNSEEEEEDALTLSSAMWFSWGVLLNSGIGEGAPRSFSARILGMVWAGFAMIIVASYTANLAAFLVLDRPEERITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSRSNAPATLTFENMAGVFMLVAGGIVAGIFLIFIEIAYKRHKDARRKQMQLAFAAVNVWRKNLQDRKSGRAEPDPKKKATFRAITSTLASSFKRRRSSKDTSTGGGRGALQNQKDTVLPRRAIEREEGQLQLCSRHRES

SEQ ID NO: 10 - protein, 557 aa, Homo sapiens
  source 1..557: note = SEQ ID NO: 2 from US 9,250,250, corresponding to LGI1
MESERSKRMGNACIPLKRIAYFLCLLSALLLTEGKKPAKPKCPAVCTCTKDNALCENARSIPRTVPPDVISLSFVRSGFTEISEGSFLFTPSLQLLLFTSNSFDVISDDAFIGLPHLEYLFIENNNIKSISRHTFRGLKSLIHLSLANNNLQTLPKDIFKGLDSLTNVDLRGNSFNCDCKLKWLVEWLGHTNATVEDIYCEGPPEYKKRKINSLSSKDFDCIITEFAKSQDLPYQSLSIDTFSYLNDEYVVIAQPFTGKCIFLEWDHVEKTFRNYDNITGTSTVVCKPIVIETQLYVIVAQLFGGSHIYKRDSFANKFIKIQDIEILKIRKPNDIETFKIENNWYFVVADSSKAGFTTIYKWNGNGFYSHQSLHAWYRDTDVEYLEIVRTPQTLRTPHLILSSSSQRPVIYQWNKATQLFTNQTDIPNMEDVYAVKHFSVKGDVYICLTRFIGDSKVMKWGGSSFQDIQRMPSRGSMVFQPLQINNYQYAILGSDYSFTQVYNWDAEKAKFVKFQELNVQAPRSFTHVSINKRNFLFASSFKGNTQIYKHVIVDLSA

SEQ ID NO: 11 - protein, 961 aa, Homo sapiens
  source 1..961: note = SEQ ID NO: 1 from US 8,685,656, corresponding to GABA B
MLLLLLLAPLFLRPPGAGGAQTPNATSEGCQIIHPPWEGGIRYRGLTRDQVKAINFLPVDYEIEYVCRGEREVVGPKVRKCLANGSWTDMDTPSRCVRICSKSYLTLENGKVFLTGGDLPALDGARVDFRCDPDFHLVGSSRSICSQGQWSTPKPHCQVNRTPHSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKIYDPSINCTVDEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHVRYIQNSQPNLNNLTAVGCSLALAAVFPLGLDGYHIGRNQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSRKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSEAQDTMKTGSSTNNNEEEKSRLLEKENRELEKIIAEKEERVSELRHQLQSRQQLRSRRHPPTPPEPSGGLPRGPPEPPDRLSCDGSRVHLLYK

SEQ ID NO: 12 - protein, 456 aa, Homo sapiens
  source 1..456: note = SEQ ID NO: 1 from US 11,041,005, corresponding to GABA A (alpha 1)
MRKSPGLSDCLWAWILLLSTLTGRSYGQPSLQDELKDNTTVFTRILDRLLDGYDNRLRPGLGERVTEVKTDIFVTSFGPVSDHDMEYTIDVFFRQSWKDERLKFKGPMTVLRLNNLMASKIWTPDTFFHNGKKSVAHNMTMPNKLLRITEDGTLLYTMRLTVRAECPMHLEDFPMDAHACPLKFGSYAYTRAEVVYEWTREPARSVVVAEDGSRLNQYDLLGQTVDSGIVQSSTGEYVVMTTHFHLKRKIGYFVIQTYLPCIMTVILSQVSFWLNRESVPARTVFGVTTVLTMTTLSISARNSLPKVAYATAMDWFIAVCYAFVFSALIEFATVNYFTKRGYAWDGKSVVPEKPKKVKDPLIKKNNTYAPTATSYTPNLARGDPGLATIAKSATIEPKEVKPETKPPEPKKTFNSVSKIDRLSRIAFPLLFGIFNLVYWATYLNREPQLKAPTPHQ

SEQ ID NO: 13 - protein, 473 aa, Homo sapiens [sequence omitted]

SEQ ID NO: 14 - protein, 475 aa, Homo sapiens [sequence omitted]

SEQ ID NO: 15 - protein, 2758 aa, Homo sapiens
MSDKMSSFLHIGDICSLYAEGSTNGFISTLGLVDDRCVVQPETGDLNNPPKKFRDCLFKLCPMNRYSAQKQFWKAAKPGANSTTDAVLLNKLHHAADLEKKQNETENRKLLGTVIQYGNVIQLLHLKSNKYLTVNKRLPALLEKNAMRVTLDEAGNEGSWFYIQPFYKLRSIGDSVVIGDKVVLNPVNAGQPLHASSHQLVDNPGCNEVNSVNCNTSWKIVLFMKWSDNKDDILKGGDVVRLFHAEQEKFLTCDEHRKKQHVFLRTTGRQSATSATSSKALWEVEVVQHDPCRGGAGYWNSLFRFKHLATGHYLAAEVDPDFEEECLEFQPSVDPDQDASRSRLRNAQEKMVYSLVSVPEGNDISSIFELDPTTLRGGDSLVPRNSYVRLRHLCTNTWVHSTNIPIDKEEEKPVMLKIGTSPVKEDKEAFAIVPVSPAEVRDLDFANDASKVLGSIAGKLEKGTITQNERRSVTKLLEDLVYFVTGGTNSGQDVLEVVFSKPNRERQKLMREQNILKQIFKLLQAPFTDCGDGPMLRLEELGDQRHAPFRHICRLCYRVLRHSQQDYRKNQEYIAKQFGFMQKQIGYDVLAEDTITALLHNNRKLLEKHITAAEIDTFVSLVRKNREPRFLDYLSDLCVSMNKSIPVTQELICKAVLNPTNADILIETKLVLSRFEFEGVSSTGENALEAGEDEEEVWLFWRDSNKEIRSKSVRELAQDAKEGQKEDRDVLSYYRYQLNLFARMCLDRQYLAINEISGQLDVDLILRCMSDENLPYDLRASFCRLMLHMHVDRDPQEQVTPVKYARLWSEIPSEIAIDDYDSSGASKDEIKERFAQTMEFVEEYLRDVVCQRFPFSDKEKNKLTFEVVNLARNLIYFGFYNFSDLLRLTKILLAILDCVHVTTIFPISKMAKGEENKGNNDVEKLKSSNVMRSIHGVGELMTQVVLRGGGFLPMTPMAAAPEGNVKQAEPEKEDIMVMDTKLKIIEILQFILNVRLDYRISCLLCIFKREFDESNSQTSETSSGNSSQEGPSNVPGALDFEHIEEQAEGIFGGSEENTPLDLDDHGGRTFLRVLLHLTMHDYPPLVSGALQLLFRHFSQRQEVLQAFKQVQLLVTSQDVDNYKQIKQDLDQLRSIVEKSELWVYKGQGPDETMDGASGENEHKKTEEGNNKPQKHESTSSYNYRVVKEILIRLSKLCVQESASVRKSRKQQQRLLRNMGAHAVVLELLQIPYEKAEDTKMQEIMRLAHEFLQNFCAGNQQNQALLHKHINLFLNPGILEAVTMQHIFMNNFQLCSEINERVVQHFVHCIETHGRNVQYIKFLQTIVKAEGKFIKKCQDMVMAELVNSGEDVLVFYNDRASFQTLIQMMRSERDRMDENSPLMYHIHLVELLAVCTEGKNVYTEIKCNSLLPLDDIVRVVTHEDCIPEVKIAYINFLNHCYVDTEVEMKEIYTSNHMWKLFENFLVDICRACNNTSDRKHADSILEKYVTEIVMSIVTTFFSSPFSDQSTTLQTRQPVFVQLLQGVFRVYHCNWLMPSQKASVESCIRVLSDVAKSRAIAIPVDLDSQVNNLFLKSHSIVQKTAMNWRLSARNAARRDSVLAASRDYRNIIERLQDIVSALEDRLRPLVQAELSVLVDVLHRPELLFPENTDARRKCESGGFICKLIKHTKQLLEENEEKLCIKVLQTLREMMTKDRGYGEKLISIDELDNAELPPAPDSENATEELEPSPPLRQLEDHKRGEALRQVLVNRYYGNVRPSGRRESLTSFGNGPLSAGGPGKPGGGGGGSGSSSMSRGEMSLAEVQCHLDKEGASNLVIDLIMNASSDRVFHESILLAIALLEGGNTTIQHSFFCRLTEDKKSEKFFKVFYDRMKVAQQEIKATVTVNTSDLGNKKKDDEVDRDAPSRKKAKEPTTQITEEVRDQLLEASAATRKAFTTFRREADPDDHYQPGEGTQATADKAKDDLEMSAVITIMQPILRFLQLLCENHNRDLQNFLRCQNNKTNYNLVCETLQFLDCICGSTTGGLGLLGLYINEKNVALINQTLESLTEYCQGPCHENQNCIATHESNGIDIITALILNDINPLGKKRMDLVLELKNNASKLLLAIMESRHDSENAERILYNMRPKELVEVIKKAYMQGEVEFEDGENGEDGAASPRNVGHNIYILAHQLARHNKELQSMLKPGGQVDGDEALEFYAKHTAQIEIVRLDRTMEQIVFPVPSICEFLTKESKLRIYYTTERDEQGSKINDFFLRSEDLFNEMNWQKKLRAQPVLYWCARNMSFWSSISFNLAVLMNLLVAFFYPFKGVRGGTLEPHWSGLLWTAMLISLAIVIALPKPHGIRALIASTILRLIFSVGLQPTLFLLGAFNVCNKIIFLMSFVGNCGTFTRGYRAMVLDVEFLYHLLYLVICAMGLFVHEFFYSLLLFDLVYREETLLNVIKSVTRNGRSIILTAVLALILVYLFSIVGYLFFKDDFILEVDRLPNETAVPETGESLASEFLFSDVCRVESGENCSSPAPREELVPAEETEQDKEHTCETLLMCIVTVLSHGLRSGGGVGDVLRKPSKEEPLFAARVIYDLLFFFMVIIIVLNLIFGVIIDTFADLRSEKQKKEEILKTTCFICGLERDKFDNKTVTFEEHIKEEHNMWHYLCFIVLVKVKDSTEYTGPESYVAEMIKERNLDWFPRMRAMSLVSSDSEGEQNELRNLQEKLESTMKLVTNLSGQLSELKDQMTEQRKQKQRIGLLGHPPHMNVNPQQPA

SEQ ID NO: 16 - protein, 195 aa, Homo sapiens
  source 1..195: note = SEQ ID NO: 7 from US 6,387,639, corresponding to MA2
PLALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEALSPATVPCISPELLAHLLGQAMAHAPQPLLPMRYRKLRVFSGSAVPAPEEESFEVWLEQATEIVKEWP

SEQ ID NO: 17 - protein, 336 aa, Homo sapiens
  source 1..336: note = SEQ ID NO: 1 from US 10,962,554, corresponding to IGLON5
MPPPAPGARLRLLAAAALAGLAVISRGLLSQSLEFNSPADNYTVCEGDNATLSCFIDEHVTRVAWLNRSNILYAGNDRWTSDPRVRLLINTPEEFSILITEVGLGDEGLYTCSFQTRHQPYTTQVYLIVHVPARIVNISSPVTVNEGGNVNLLCLAVGRPEPTVTWRQLRDGFTSEGEILEISDIQRGQAGEYECVTHNGVNSAPDSRRVLVTVNYPPTITDVTSARTALGRAALLRCEAMAVPPADFQWYKDDRLLSSGTAEGLKVQTERTRSMLLFANVSARHYGNYTCRAANRLGASSASMRLLRPGSLENSAPRPPGLLALLSALGWLWWRM

SEQ ID NO: 18 - protein, 801 aa, Homo sapiens
  source 1..801: note = SEQ ID NO: 1 from US 9,719,993, corresponding to DPPX
MKEKAMIKTAKMQGNVMELVGSNPPQRNWKGIAIALLVILVICSLIVTSVILLTPAEDNSLSQKKKVTVEDLFSEDFKIHDPEAKWISDTEFIYREQKGTVRLWNVETNTSTVLIEGKKIESLRAIRYEISPDREYALFSYNVEPIYQHSYTGYYVLSKIPHGDPQSLDPPEVSNAKLQYAGWGPKGQQLIFIFENNIYYCAHVGKQAIRVVSTGKEGVIYNGLSDWLYEEEILKTHIAHWWSPDGTRLAYAAINDSRVPIMELPTYTGSIYPTVKPYHYPKAGSENPSISLHVIGLNGPTHDLEMMPPDDPRMREYYITMVKWATSTKVAVTWLNRAQNVSILTLCDATTGVCTKKHEDESEAWLHRQNEEPVFSKDGRKFFFIRAIPQGGRGKFYHITVSSSQPNSSNDNIQSITSGDWDVTKILAYDEKGNKIYFLSTEDLPRRRQLYSANTVGNFNRQCLSCDLVENCTYFSASFSHSMDFFLLKCEGPGVPMVTVHNTTDKKKMFDLETNEHVKKAINDRQMPKVEYRDIEIDDYNLPMQILKPATFTDTTHYPLLLVVDGTPGSQSVAEKFEVSWETVMVSSHGAVVVKCDGRGSGFQGTKLLHEVRRRLGLLEEKDQMEAVRTMLKEQYIDRTRVAVFGKDYGGYLSTYILPAKGENQGQTFTCGSALSPITDFKLYASAFSERYLGLHGLDNRAYEMTKVAHRVSALEEQQFLIIHPTADEKIHFQHTAELITQLIRGKANYSLQIYPDESHYFTSSSLKQHLYRSIINFFVECFRIQDKLLTVTAKEDEEED

(19) United States
(12) Patent Application Publication
(10) Pub. No.: US 2023/0053910 A1
(43) Pub. Date: Feb. 23, 2023

(54) Method and reagents for the detection of an autoantibody

(71) Applicants: Institut d'investigacions Biomèdiques August Pi i Sunyer (IDIBAPS), Barcelona (ES); Institució Catalana de Recerca i Estudis Avançats (ICREA), Barcelona (ES); Fundació Clinic per a la Recerca Biomèdica (FCRB), Barcelona (ES)

(72) Inventors: Josep Dalmau, Barcelona (ES); Lidia Sabater, Barcelona (ES); Francesc Graus, Barcelona (ES)

(73) Assignees: Institut d'investigacions Biomèdiques August Pi i Sunyer (IDIBAPS), Barcelona (ES); Institució Catalana de Recerca i Estudis Avançats (ICREA), Barcelona (ES); Fundació Clinic per a la Recerca Biomèdica (FCRB), Barcelona (ES)

(21) Appl. No.: 17/818,802

(22) Filed: Aug. 10, 2022

(30) Foreign Application Priority Data
Aug. 10, 2021 (EP) 21190690.4

(51) Int. Cl. G01N 33/564; G01N 33/68

(52) U.S. Cl. CPC G01N 33/564; G01N 33/6854

(57) ABSTRACT

A method involves detecting in a sample an autoantibody binding specifically to one or more from Gluk1, Gluk2, Gluk3, Gluk4 and Gluk5, preferably Gluk2. An autoantibody binds specifically to one or more from Gluk1, Gluk2, Gluk3, Gluk4 and Gluk5, preferably Gluk2. A diagnostically useful carrier with a solid phase with an immobilized polypeptide contains one or more from Gluk1, Gluk2, Gluk3, Gluk4 and Gluk5, preferably Gluk2 or a variant thereof. The autoantibody can be used for diagnosing a neurological autoimmune disease or a cancer.

### CROSS-REFERENCE TO RELATED APPLICATIONS

This application claims priority to European Application No. 21190690.4, filed on Aug. 10, 2021, the content of which is hereby incorporated by reference in its entirety.

### REFERENCE TO A SEQUENCE LISTING

The present application is accompanied by an XML file as a computer readable form containing the sequence listing entitled, “004088US_SL”, created on Aug. 8, 2022, with a file size of 32,395 bytes, the content of which is hereby incorporated by reference in its entirety.

### BACKGROUND OF THE INVENTION

### Field of the Invention

The present invention relates to a method comprising the step detecting in a sample an autoantibody binding specifically to one or more from the group comprising Gluk1, Gluk2, Gluk3, Gluk4, and Gluk5, preferably Gluk2, an autoantibody binding specifically to one or more from the group comprising Gluk1, Gluk2, Gluk3, Gluk4 and Gluk5, preferably Gluk2, a diagnostically useful carrier with a solid phase with an immobilized polypeptide comprising one or more from the group comprising Gluk1, Gluk2, Gluk3, Gluk4 and Gluk5, preferably Gluk2 or a variant thereof and a use of the autoantibody for diagnosing a neurological autoimmune disease or a cancer.

### Description of Related Art

Neurological autoimmune diseases are rare but are potentially treatable. Their hallmark it the occurrence of neurological symptoms and an autoimmune response, usually the emergence of an autoantibody binding specifically to structures in the brain such as polypeptides having important functions for neurotransmission. They can affect any area of the nervous system, including the central, peripheral and autonomic nervous system. Although the system involvement is often multifocal, like encephalomyelitis, it can involve a single system, e. g. cerebellar degeneration.

Autoimmune encephalitis is major neurological autoimmune disease, in particular anti-NMDA receptor autoimmune encephalitis which affects 1.5 patient per million per year. Symptoms such as paranoia, psychosis and violent behavior usually appear psychiatric in nature at first, but as the disease progresses, this may be accompanied by seizures, impaired cognition, memory deficit and speech problems. At a later stage treatment in an intensive care unit may be required if patients suffer from medically urgent symptoms including cerebellar ataxia, autonomic dysfunction and catatonia. An autoantibody to the NR1 subunit of the NMDA receptor was discovered as the cause (Dalmau et al. (2008): Anti-NMDA-receptor encephalitis: case series and analysis of the effects of antibodies, Lancet Neurology, Volume 7, Issue 12).

Early diagnosis and treatment of neurological autoimmune diseases is important because any delay can result in rapid progression and irreversible neurological damage. By contrast, early and appropriate treatment, usually by some form of immunosuppressive therapy, may reverse some of the damages. In some cases, a complete recovery is possible. For example, 80% of patients suffering from NMDA receptor encephalitis have a good outcome with treatment, although long-term mental problems or a recurrence are possible.

Diagnosing neurological autoimmune diseases is often difficult, though. One of the reasons is the absence of a particular clinical pattern and absence of specific imaging and laboratory abnormalities. A combination of clinical and laboratory evaluations has to be deployed to reach diagnosis early. Treatment of underlying cancer, if present, is important in the treatment of the neurological condition.

Neurological autoantibodies are helpful for the diagnosis. Their presence, alone or in combination with other indicators, helps to establish the autoimmune nature of the disease, helping the clinician to differentiate the new neurological symptoms of a neurological autoimmune disease from a neurological condition which is the result of an infection, treatment-related-complications like toxic neuropathies drug abuse and psychiatric diseases. Moreover, they are of help in detecting the recurrence of the disease in already seropositive patients.

However, many patients suspected of suffering from a neurological autoimmune disease will not have any antibodies detectable by state-of-the-art tests, not in the least because many diagnostically relevant antibodies are still unknown. There is the danger that these patients may be undiagnosed or even be misdiagnosed. In the past, some of them have been sent to psychiatric wards while, with appropriate immunosuppressive treatment, they could have led reasonably normal lives.

### SUMMARY OF THE INVENTION

Therefore, the problem underlying the present invention is to provide reagents and methods for the diagnosis of a neurological autoimmune disease and/or cancer, preferably associated with the presence of an autoantibody binding specifically to one or more from the group comprising Gluk1, Gluk2, Gluk3, Gluk4 and Gluk5, preferably Gluk2.

Another problem underlying the present invention is to distinguish an autoimmune encephalitis and an encephalitis caused by an infection.

Another problem underlying the present invention is to provide an assay with an increased diagnostic reliability, in particular with regard to specificity and/or sensitivity, for the diagnosis for a neurological autoimmune disease or cancer, optionally in combination with state-of-the-art assays.

The problem underlying the present invention is solved by the subject matter as described below.

In a first aspect, the problem underlying the present invention is solved by a method comprising the step detecting in a sample an autoantibody binding specifically to one or more from the group comprising Gluk1, Gluk2, Gluk3, Gluk4 and Gluk5, preferably Gluk2.

In a second aspect, the problem underlying the present invention is solved by a method for isolating an antibody binding to one or more from the group comprising Gluk1, Gluk2, Gluk3, Gluk4 and Gluk5, preferably Gluk2, comprising the steps
a) immobilizing on a carrier one or more polypeptides each comprising one or more antigens from the group comprising Gluk1, Gluk2, Gluk3, Gluk4 and Gluk5, preferably Gluk2 or a variant thereof,
b) contacting a sample comprising antibodies with the polypeptide(s) under conditions compatible with formation of a complex, wherein said antibody binds to said polypeptide,
c) separating the complex formed in step a) from the sample, and
d) separating the antibody from the polypeptide.

In a third aspect, the problem underlying the present invention is solved by a method comprising the steps
a) immobilizing one or more polypeptide(s) each comprising one or more antigens from the group comprising Gluk1, Gluk2, Gluk3, Gluk4 and Gluk5, preferably Gluk2 or a variant thereof on a carrier,
b) contacting the carrier with a liquid, wherein a candidate drug is present and/or the liquid does not comprise a sample from a subject to be diagnosed, comprising an antibody binding specifically to one or more from the group comprising Gluk1, Gluk2, Gluk3, Gluk4 and Gluk5, preferably Gluk2,
c) contacting the carrier with a means for detecting an immobilized antibody, and
d) detecting the presence, preferably in quantitative manner.

In a fourth aspect, the problem is solved by an autoantibody binding specifically to one or more, preferably one antigen from the group comprising Gluk1, Gluk2, Gluk3, Gluk4 and Gluk5, preferably Gluk2, more preferably in a solution comprising one or more, more preferably all from the group comprising an artificial buffer, a preservative and an artificial anticoagulant.

In a fifth aspect, a diagnostically useful carrier with a solid phase with one or more polypeptides each comprising one or more antigens from the group comprising Gluk1, Gluk2, Gluk3, Gluk4 and Gluk5, preferably Gluk2 or a variant thereof, immobilized on the solid phase, and a) a negative control and/or b) at least one additional diagnostically useful antigen, wherein the polypeptide and the negative control or additional antigen are spatially separate on the carrier, or

a first diagnostically useful carrier with a solid phase comprising one or more polypeptides each comprising one or more antigens from the group comprising Gluk1, Gluk2, Gluk3, Gluk4 and Gluk5, preferably Gluk2 or a variant thereof, immobilized on the solid phase, and a second diagnostically useful carrier comprising a solid phase comprising an immobilized a) negative control and/or b) at least one additional immobilized polypeptide comprising an antigen or a variant thereof.

In a preferred embodiment, the at least one additional antigen is at least one antigen, preferably all antigens from the group comprising NMDAR, LgI1, AMPA1, AMPA2, CASPR2, GABA B, GABA A, DPPX, IGLON5, Hu, Yo, CV2/CRMP5, Ri, Ma2, Amphiphysin, Recoverin, RGS8, DAGLA, STX1B, AK5, AP3B2, Flotillin1+2, GRM1, GRM2, GRM5, GLURD2, ITPR1, KCNA2, NCDN, Septin 3+5+6+7+11 and Sez6L2.

In a sixth aspect, the problem is solved by a use of a) the autoantibody according to the present invention or b) of the combination of one or more from the group comprising Gluk1, Gluk2, Gluk3, Gluk4 and Gluk5, preferably Gluk2 and a means for detecting or capturing an autoantibody, preferably IgG antibody or c) the carrier according to the present invention for diagnosing a neurological autoimmune disease or a cancer.

In a seventh aspect, the problem is solved by a kit comprising a polypeptide comprising one or more from the group comprising Gluk1, Gluk2, Gluk3, Gluk4 and Gluk5, preferably Gluk2 or a variant thereof and a diagnostically useful carrier, preferably according to the present invention, wherein
a) the polypeptide is immobilized on a diagnostically useful carrier, preferably according to the present invention, and the kit further comprises a means for detecting an autoantibody, preferably immobilized, binding specifically to one or more from the group comprising Gluk1, Gluk2, Gluk3, Gluk4 and Gluk5, preferably Gluk2,
b) the polypeptide and the carrier are configured for immobilizing the polypeptide on the surface of the carrier, preferably via an affinity tag and a ligand binding to the affinity tag, and the kit further comprises a means for detecting an immobilized antibody binding specifically to one or more from the group comprising Gluk1, Gluk2, Gluk3, Gluk4 and Gluk5, preferably Gluk2,
c) the carrier is immobilized with a means for capturing an antibody and the kit further comprises a means for detecting a captured antibody binding specifically to the transporter, which is preferably polypeptide comprising a detectable label,
d) the carrier and a means for capturing an antibody are configured for immobilizing the means for capturing an antibody on the surface of the carrier, preferably via an affinity tag and a ligand binding to the affinity tag, and the kit further comprises a means for detecting a captured antibody binding specifically to one or more from the group comprising Gluk1, Gluk2, Gluk3, Gluk4 and Gluk5, preferably Gluk2, which is preferably the polypeptide comprising a detectable label

and preferably one or more, more preferably all from the group comprising a recombinant antibody binding to one or more from the group comprising Gluk1, Gluk2, Gluk3, Gluk4 and Gluk5, preferably Gluk2 or a variant thereof, an isolated autoantibody binding to one or more from the group comprising Gluk1, Gluk2, Gluk3, Gluk4 and Gluk5, preferably Gluk2, a chemical solution reactive with a detectable label, a positive control, a negative control, a water-tight vessel for incubating a sample with a carrier or reagent, a wash buffer, and a calibrator, preferably a set of calibrators.

In an eighth aspect, the problem is solved by a use of a polypeptide comprising one or more from the group comprising Gluk1, Gluk2, Gluk3, Gluk4 and Gluk5, preferably Gluk2 or a variant thereof or an autoantibody binding specifically to one or more from the group comprising Gluk1, Gluk2, Gluk3, Gluk4 and Gluk5, preferably Gluk2 or a recombinant antibody binding specifically to a polypeptide from the group comprising Gluk1, Gluk2, Gluk3, Gluk4 and Gluk5, preferably Gluk2, for the manufacture of a kit or medical device, preferably diagnostic device, for the diagnosis of a disease.

In a ninth aspect, the problem is solved by a use of an autoantibody binding specifically to one or more from the group comprising Gluk1, Gluk2, Gluk3, Gluk4 and Gluk5, preferably Gluk2 or a recombinant antibody binding specifically to one or more from the group comprising Gluk1, Gluk2, Gluk3, Gluk4 and Gluk5, preferably Gluk2, which is preferably recognized by secondary antibodies to human IgG class immunoglobulins, as a positive control for the detection of an autoantibody binding specifically to one or more from the group comprising Gluk1, Gluk2, Gluk3, Gluk4 and Gluk5, preferably Gluk2 in a sample.

In a tenth aspect, the problem is solved by an ex vivo method for removing an autoantibody binding specifically to one or more from the group comprising Gluk1, Gluk2, Gluk3, Gluk4 and Gluk5, preferably Gluk2 from blood, preferably serum of a patient.

In a preferred embodiment, the polypeptide is a recombinant, isolated and/or purified polypeptide.

In a preferred embodiment, the antibody or autoantibody is a mammalian, preferably human autoantibody or the sample is a mammalian, preferably human sample comprising a representative set of antibodies, preferably selected from the group comprising whole blood, plasma, serum, cerebrospinal fluid and saliva.

In a preferred embodiment, the autoantibody or complex is detected using a detection method selected from the group comprising immunodiffusion, immunoelectrophoresis, light scattering immunoassays, agglutination, labeled immunoassays such as those from the group comprising radiolabeled immunoassay, enzyme immunoassays, more preferably ELISA, chemiluminescence immunoassays, preferably electrochemiluminescence immunoassay, and immunofluorescence, preferably indirect immunofluorescence.

In a preferred embodiment, the carrier is selected from the group comprising a glass slide, preferably for microscopy, a biochip, a microtiter plate, a lateral flow device, a test strip, a membrane, preferably a line blot, a chromatography column and a bead, preferably a magnetic or fluorescent bead.

The invention also includes the following embodiments:

1. A method comprising the step detecting in a sample, preferably a human sample, an autoantibody binding specifically to one or more from the group comprising Gluk1, Gluk2, Gluk3, Gluk4 and Gluk5, preferably Gluk2.

2. A method for isolating an antibody binding to a one or more from the group comprising Gluk1, Gluk2, Gluk3, Gluk4 and Gluk5, preferably Gluk2, comprising the steps

a) immobilizing on a carrier one or more polypeptides each comprising a one or more antigen from the group comprising Gluk1, Gluk2, Gluk3, Gluk4 and Gluk5, preferably Gluk2, or a variant thereof,

b) contacting a sample comprising antibodies with the polypeptide under conditions compatible with formation of a complex, wherein said antibody binds to said polypeptide,

c) separating the complex formed in step a) from the sample, and

d) separating the antibody from the polypeptide.

3. A method comprising the steps

a) immobilizing one or more polypeptides each comprising one or more antigen from the group comprising Gluk1, Gluk2, Gluk3, Gluk4 and Gluk5, preferably Gluk2 or a variant thereof on a carrier,

b) contacting the carrier with a liquid, wherein a candidate drug is present and/or the liquid does not comprise a sample from a subject to be diagnosed, comprising an antibody binding specifically to Gluk1, Gluk2, Gluk3, Gluk4 and Gluk5, preferably Gluk2,

c) contacting the carrier with a means for detecting an immobilized antibody, and

d) detecting the presence, preferably in quantitative manner.

4. An autoantibody binding specifically to one or more from the group comprising Gluk1, Gluk2, Gluk3, Gluk4 and Gluk5, preferably Gluk2, which is preferably in a solution comprising one or more, more preferably all from the group comprising an artificial buffer, a preservative and an artificial anticoagulant.

5. A diagnostically useful carrier with a solid phase with one or more polypeptides each comprising one or more antigen from the group comprising Gluk1, Gluk2, Gluk3, Gluk4 and Gluk5, preferably Gluk2 or a variant thereof, immobilized on the solid phase, and a) a negative control and/or b) at least one additional diagnostically useful antigen, wherein the polypeptide and the negative control or additional antigen are spatially separate on the carrier,

or a first diagnostically useful carrier with a solid phase comprising one or more polypeptides each comprising one or more antigen from the group comprising Gluk1, Gluk2, Gluk3, Gluk4 and Gluk5, preferably Gluk2 or a variant thereof, immobilized on the solid phase, and a second diagnostically useful carrier comprising a solid phase with an immobilized a) negative control and/or b) at least one additional immobilized polypeptide comprising an antigen or a variant thereof.

6. The carrier according to embodiment 5, wherein the at least one additional antigen is at least one antigen, preferably all antigens from the group comprising NMDAR, LgI1, AMPA1, AMPA2, CASPR2, GABA B, GABA A, DPPX, IGLON5, Hu, Yo, CV2/CRMP5, Ri, Ma2, Amphiphysin, Recoverin, RGS8, DAGLA, STX1B, AK5, AP3B2, Flotillin1+2, GRM1, GRM2, GRM5, GLURD2, ITPR1, KCNA2, NCDN, Septin 3+5+6+7+11, GLUK2 and Sez6L2 or a variant thereof, preferably from the group comprising NMDA, AMPA1, AMPA2, CASPR2, LGI1, IGLON5, DPPX and GABA B or a variant thereof, more preferably NMDAR or a variant thereof.

7. A use of a) the autoantibody according to embodiment 4 or b) of the combination of one or more polypeptides each comprising one or more antigens from the group comprising Gluk1, Gluk2, Gluk3, Gluk4 and Gluk5, preferably Gluk2, or a variant thereof and a means for detecting or capturing an autoantibody or c) the carrier according to any of embodiments 5 to 6 for diagnosing a neurological autoimmune disease or a cancer.

8. A kit comprising a polypeptide comprising one or more from the group comprising Gluk1, Gluk2, Gluk3, Gluk4 and Gluk5, preferably Gluk2 or a variant thereof and a diagnostically useful carrier, preferably according to any of embodiments 5 to 6, wherein

a) the polypeptide is immobilized on a diagnostically useful carrier, preferably according to any of embodiments 5 to 6, and the kit further comprises a means for detecting an autoantibody, preferably immobilized, binding specifically to one or more from the group comprising Gluk1, Gluk2, Gluk3, Gluk4 and Gluk5, preferably Gluk2,

b) the polypeptide and the carrier are configured for immobilizing the polypeptide on the surface of the carrier, preferably via an affinity tag and a ligand binding to the affinity tag, and the kit further comprises a means for detecting an immobilized antibody binding specifically to one or more from the group comprising Gluk1, Gluk2, Gluk3, Gluk4 and Gluk5, preferably Gluk2,

c) the carrier is immobilized with a means for capturing an antibody and the kit further comprises a means for detecting an immobilized antibody binding specifically to one or more from the group comprising Gluk1, Gluk2, Gluk3, Gluk4 and Gluk5, preferably Gluk2, preferably the polypeptide comprising a detectable label, or

d) the carrier and a means for capturing an antibody are configured for immobilizing the means for capturing an antibody on the surface of the carrier, preferably via an affinity tag and a ligand binding to the affinity tag, and the kit further comprises a means for detecting an immobilized antibody binding specifically to one or more from the group comprising Gluk1, Gluk2, Gluk3, Gluk4 and Gluk5, preferably Gluk2, preferably the polypeptide comprising a detectable label

and preferably one or more, more preferably all from the group comprising a recombinant antibody binding to one or more from the group comprising Gluk1, Gluk2, Gluk3, Gluk4 and Gluk5, preferably Gluk2 or a variant thereof, an isolated autoantibody binding to one or more from the group comprising Gluk1, Gluk2, Gluk3, Gluk4 and Gluk5, preferably Gluk2, a chemical solution reactive with a detectable label, a positive control, a negative control, a water-tight vessel for incubating a sample with a carrier or reagent, a wash buffer, and a calibrator, preferably a set of calibrators.

9. A use of a polypeptide comprising one or more from the group comprising Gluk1, Gluk2, Gluk3, Gluk4 and Gluk5, preferably Gluk2 or a variant thereof, or an autoantibody binding specifically to one or more from the group comprising Gluk1, Gluk2, Gluk3, Gluk4 and Gluk5, preferably Gluk2, or a recombinant antibody binding specifically to one or more from the group comprising Gluk1, Gluk2, Gluk3, Gluk4 and Gluk5, preferably Gluk2, for the manufacture of a kit or medical device, preferably diagnostic device, for the diagnosis of a disease.

10. A use of an autoantibody binding specifically to one or more from the group comprising Gluk1, Gluk2, Gluk3, Gluk4 and Gluk5, preferably Gluk2 or a recombinant antibody binding specifically to one or more from the group comprising Gluk1, Gluk2, Gluk3, Gluk4 and Gluk5, preferably Gluk2, which is preferably recognized by secondary antibodies to human IgG class immunoglobulins, as a positive control for the detection of an antibody binding specifically to one or more from the group comprising Gluk1, Gluk2, Gluk3, Gluk4 and Gluk5, preferably Gluk2 in a sample.

11. An ex vivo method for removing an autoantibody binding specifically to one or more from the group comprising Gluk1, Gluk2, Gluk3, Gluk4 and Gluk5, preferably Gluk2 from blood, preferably serum of a patient.

12. The method, carrier, use or kit according to any of the preceding embodiments, wherein the polypeptide is a recombinant, isolated and/or purified polypeptide.

13. The method, carrier, use or kit according to any of the preceding embodiments, wherein the antibody or autoantibody is a mammalian, preferably human autoantibody or the sample is a mammalian, preferably human sample comprising a representative set of antibodies, preferably selected from the group comprising whole blood, plasma, serum, cerebrospinal fluid and saliva.

14. The method, carrier, use or kit according to any of the preceding embodiments, wherein the autoantibody or complex is detected using a detection method selected from the group comprising immunodiffusion, electrophoresis, light scattering immunoassays, agglutination, labeled immunoassays such as those from the group comprising radiolabeled immunoassay, enzyme immunoassays, more preferably ELISA, chemiluminescence immunoassays, preferably electrochemiluminescence immunoassay, and immunofluorescence, preferably indirect immunofluorescence.

15. The method, carrier, use or kit according to any of the preceding embodiments, wherein the carrier is selected from the group comprising a glass slide, preferably for microscopy, a biochip, a microtiter plate, a lateral flow device, a test strip, a membrane, preferably a line blot, a chromatography column and a bead, preferably a magnetic or fluorescent bead.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a first section of brain immunostaining with CSF from a patient of case 1.

FIG. 1B shows a second section of brain immunostaining with CSF from a patient of case 1.

FIG. 1C shows a third section of brain immunostaining with CSF from a patient of case 1.

FIG. 1D shows a first section of brain immunostaining with CSF from a patient of case 2.

FIG. 1E shows a second section of brain immunostaining with CSF from a patient of case 2.

FIG. 1F shows a third section of brain immunostaining with CSF from a patient of case 2.

FIG. 1G shows a first section of brain immunostaining with CSF from a patient with antibodies against AMPAR and GluK2.

FIG. 1H shows a second section of brain immunostaining with CSF from a patient with antibodies against AMPAR and GluK2.

FIG. 1I shows a third section of brain immunostaining with CSF from a patient with antibodies against AMPAR and GluK2.

FIG. 1J shows a first section of brain immunostaining with CSF from a patient with anti-AMPAR.

FIG. 1K shows a second section of brain immunostaining with CSF from a patient with anti-AMPAR.

FIG. 1L shows a third section of brain immunostaining with CSF from a patient with anti-AMPAR.

FIG. 1M shows a first section of brain immunostaining with CSF from a patient with NMDAR encephalitis.

FIG. 1N shows a second section of brain immunostaining with CSF from a patient with NMDAR encephalitis.

FIG. 1O shows a third section of brain immunostaining with CSF from a patient with NMDAR encephalitis.

FIG. 2A shows an axial brain MRI of patient 1 at symptom onset.

FIG. 2B shows a sagittal brain MRI of patient 1 at symptom onset.

FIG. 2C shows an axial brain MRI of patient 1 two weeks later.

FIG. 2D shows a sagittal brain MRI of patient 1 two weeks later.

FIG. 2E shows an axial brain MRI of patient 1 at a last follow-up.

FIG. 2F shows a first axial brain MRI of patient 2 at symptom onset.

FIG. 2G shows a second axial brain MRI of patient 2 at symptom onset.

FIG. 2H shows a third axial brain MRI of patient 2 at symptom onset.

FIG. 2I shows a fourth axial brain MRI of patient 2 at symptom onset.

FIG. 2J shows an axial brain MRI of patient 2 after 2 years later.

FIG. 2K shows a first axial brain MRI of patient 5 at symptom onset.

FIG. 2L shows a second axial brain MRI of patient 5 at symptom onset.

FIG. 2M shows a third axial brain MRI of patient 5 at symptom onset.

FIG. 2N shows a fourth axial brain MRI of patient 5 at symptom onset.

FIG. 2O shows an axial brain MRI of patient 5 after 5 weeks later.

FIG. 2P shows an axial brain MRI of patient 4 at presentation.

FIG. 2Q shows a first sagittal brain MRI of patient 4 at presentation.

FIG. 2R shows a second sagittal brain MRI of patient 4 at presentation.

FIG. 2S shows an axial brain MRI of patient 4 after disease onset.

FIG. 2T shows a sagittal brain MRI of patient 4 after disease onset.

FIG. 3A shows an immunoblot of immunoprecipitation of GluK2.

FIG. 3B shows a cell-based assay with HEK293 cells expressing GluK2 immunolabelled with patient's serum antibodies.

FIG. 3C shows a cell-based assay with a commercial antibody against Myc-tag to confirm the expression of GluK2.

FIG. 3D shows the merged reactivities of FIGS. 3B and 3C.

FIG. 3E shows a cell-based assay using serum from a healthy subject that demonstrates lack of reactivity with GluK2.

FIG. 3F shows a cell-based assay with a commercial antibody against Myc-tag to confirm the lack of reactivity with GluK2.

FIG. 3G shows the merged reactivities of FIGS. 3E and 3F.

FIG. 3H shows an immunoblot of immunoprecipitation of GluK2 from live HEK293 cells expressing GluK2 and patients' or control sera.

FIG. 3I shows an immunostaining of the cerebellum of a wild-type mouse with CSF from patient 1.

FIG. 3J shows an immunostaining of the cerebellum of a wild-type mouse with CSF from patient 3.

FIG. 3K shows an immunostaining of the cerebellum of a wild-type mouse with CSF from patient 5.

FIG. 3L shows an immunostaining of the cerebellum of a wild-type mouse with CSF from patient 10.

FIG. 3M shows an immunostaining of the cerebellum of a wild-type mouse with CSF from patient 14.

FIG. 3N shows an immunostaining of the cerebellum of a GluK2 knockout mouse with CSF from patient 1.

FIG. 3O shows an immunostaining of the cerebellum of a GluK2 knockout mouse with CSF from patient 3.

FIG. 3P shows an immunostaining of the cerebellum of a GluK2 knockout mouse with CSF from patient 5.

FIG. 3Q shows an immunostaining of the cerebellum of a GluK2 knockout mouse with CSF from patient 10.

FIG. 3R shows an immunostaining of the cerebellum of a GluK2 knockout mouse with CSF from patient 14.

FIG. 4A shows a patient's serum reactivity with cerebellum, corresponding to patient's serum preabsorbed with HEK293 cells not expressing GluK2.

FIG. 4B shows a patient's serum reactivity with cerebellum, corresponding to patient's serum preabsorbed with HEK293 cells expressing GluK2.

FIG. 4C shows a patient's serum reactivity with live hippocampal neurons, corresponding to patient's serum preabsorbed with HEK293 cells not expressing GluK2.

FIG. 4D shows a patient's serum reactivity with live hippocampal neurons, corresponding to patient's serum preabsorbed with HEK293 cells expressing GluK2.

FIG. 4E shows a patient's serum reactivity with live cell-based assay expressing GluK2, corresponding to patient's serum preabsorbed with HEK293 cells not expressing GluK2.

FIG. 4F shows a patient's serum reactivity with live cell-based assay expressing GluK2, corresponding to patient's serum preabsorbed with HEK293 cells expressing GluK2.

FIG. 5A shows a progressive decrease of GluK2 clusters in representative dendrites of cultures of rat hippocampal neurons caused by the CSF of a representative patient (case 1), but not control CSF.

FIG. 5B shows a graph quantifying the effects using CSF from patient 1 on total neuronal surface.

FIG. 5C shows a graph quantifying the effects using CSF from patient 1 on PSD95.

FIG. 5D shows a graph quantifying the effects using CSF from patient 1 on synaptic GluK2.

FIG. 5E shows a graph quantifying the effects using CSF from patient 5 on total neuronal surface.

FIG. 5F shows a graph quantifying the effects using CSF from patient 5 on PSD95.

FIG. 5G shows a graph quantifying the effects using CSF from patient 5 on synaptic GluK2.

FIG. 6A shows current responses of cells treated for 30 minutes and current responses of cells treated for 5 hours with the indicated samples.

FIG. 6B shows the average and S.E.M. of glutamate-evoked normalized peak currents for cells untreated (basal), incubated with control serum, or 2 patients' serums.

FIG. 6C shows binding of patients' antibodies to HEK cells expressing GluK2 after incubations of 30 minutes and 5 hours.

### DETAILED DESCRIPTION OF THE INVENTION

The present invention is based on the inventors' surprising finding that antibodies to Gluk1, Gluk2, Gluk3, Gluk4 and Gluk5 exist and can be detected in samples from patients suffering from a neurological autoimmune disease and/or cancer, but not in samples from healthy subjects. Hence, they can be used to aid in the diagnosis of diseases or to diagnose diseases.

The kainate receptors are tetrameric ionotropic glutamate receptors that may include GluK1, GluK2, GluK3, GluK4 or GluK5, previously known as GluR5, GluR6, GluR7, KA1 and KA2. GluK1, GluK2 and GluK3 form functional homo- and heterotetrametric receptors, whereas GluK4 and GluK5 form functional receptors only when co-expressed with GluK1 to GluK3. As in the case of the GluA2 subunit of the AMPARs, GluK1 and GluK2 present Q/R editing at the second transmembrane domain (TM2). This Q-to-R substitution abolishes Ca²⁺ permeability while increases Cl⁻ permeation of the channel. In addition, Q residues account for larger conductance and inward rectification.

The kainate receptors are unconventional members of the glutamate receptor family in that, different from NMDAR or AMPAR, they are not predominantly found in excitatory postsynaptic complexes. In addition to function as ionotropic receptors, kainate receptors act as modulators for synaptic transmission and neuronal excitability interacting with metabotropic signalling pathways. They play a crucial role as presynaptic regulators of neurotransmitter release at both excitatory and inhibitory synapses by mechanisms not completely understood, are able to facilitate short-term and long-term plasticity and can act as modulators of GABAergic transmission.

In a preferred embodiment, Gluk1 is represented by SEQ ID NO: 1 or NP_000821.1 or P39086, preferably SEQ ID NO: 1. In a preferred embodiment, Gluk2 is represented by SEQ ID NO: 2 or NP_068775.1 or Q13002, preferably SEQ ID NO: 2. In a preferred embodiment, Gluk3 is represented by SEQ ID NO: 3 or NP_000822.2 or Q13003, preferably SEQ ID NO: 3. In a preferred embodiment, Gluk4 is represented by SEQ ID NO: 4 or NP_055434.2. In a preferred embodiment, Gluk5 is represented by SEQ ID NO: 5 or NP_002079.3. While Gluk1, Gluk2 and Gluk3 can be expressed as homomers consisting of Gluk1, Gluk2 or Gluk3 subunits only, Gluk4 and Gluk5 each need to be co-expressed as heteromers comprising a) a Gluk4 or Gluk5 subunit and b) a Gluk1, a Gluk2 or a Gluk3 subunit. The sequences referred herein represent the version available from data bases under the respective code are the ones available online on the filing or the first priority date of this application, whatever is earlier.

In a preferred embodiment, the NMDAR receptor is represented by its NR1 subunit, characterized by SEQ ID NO: 9 (corresponding to Sequence 7 of EP3505935), and this is the polypeptide to which an autoantibody to NMDAR binds or is used to detect such an autoantibody, for example in the form of a polypeptide comprised by a carrier.

In a preferred embodiment, LGI1 is characterized by SEQ ID NO: 10 (corresponding to Sequence 2 of U.S. Pat. No. 9,250,250), and this is the polypeptide to which an autoantibody to it binds or is used to detect such an autoantibody, for example in the form of a polypeptide comprised by a carrier.

In a preferred embodiment, GABA B is characterized by SEQ ID NO: 11 (B1 subunit) (corresponding to Sequence 1 of U.S. Pat. No. 8,685,656), and this is the polypeptide to which an autoantibody to it binds or is used to detect such an autoantibody, for example in the form of a polypeptide comprised by a carrier.

In a preferred embodiment, GABA A is characterized by SEQ ID NO: 12 of (alpha1) or SEQ ID NO: 13 (beta3) or SEQ ID NO: 14 (gamma2) subunit (corresponding to Sequences 1, 2, and 5 of U.S. Pat. No. 11,041,005, respectively), and these are polypeptides to which an autoantibody binds or one or more of them is used to detect such an autoantibody, for example in the form of one or more polypeptides comprised by a carrier.

In a preferred embodiment, ITPR1 is characterized by SEQ ID NO: 15 (amino acids 1-1251 of data base code Q14643 as disclosed in EP3018478), and this is the polypeptide to which an autoantibody to it binds or is used to detect such an autoantibody, for example in the form of a polypeptide comprised by a carrier.

In a preferred embodiment, MA2 is characterized by SEQ ID NO: 16 (corresponding to Sequence 7 of U.S. Pat. No. 6,387,639), and this is the polypeptide to which an autoantibody to it binds or is used to detect such an autoantibody, for example in the form of a polypeptide comprised by a carrier.

In a preferred embodiment, IGLON5 is characterized by SEQ ID NO: 17 (corresponding to Sequence 1 of U.S. Pat. No. 10,962,554), and this is the polypeptide to which an autoantibody to it binds or is used to detect such an autoantibody for example in the form of a polypeptide comprised by a carrier.

In a preferred embodiment, DPPX is characterized by SEQ ID NO: 18 (corresponding to Sequence 1 of U.S. Pat. No. 9,719,993), and this is the polypeptide to which an autoantibody to it binds or is used to detect such an autoantibody for example in the form of a polypeptide comprised by a carrier.

In a preferred embodiment, Flotillin1 and Flotillin2 are characterized by data base codes O75955 and Q14254, respectively, and these are polypeptides to which an autoantibody binds or one or more of them is used to detect such an autoantibody, for example in the form of two polypeptides comprised by a carrier.

Further antigens, to which autoantibodies bind and which may be immobilized on the carrier in addition to Gluk1, Gluk2, Gluk3, Gluk4 and Gluk5, preferably gluk2, and may be used to detect such autoantibodies, for example in the form of a polypeptide comprising an antigen, include AMPA1, AMPA2, CASPR2 (U.S. Pat. No. 9,188,587 BB), GABA B, GABA A, DPPX, IGLON5, Hu, Yo, CV2/CRMP5, Ri, Ma2, Amphiphysin, Recoverin, RGS8, DAGLA, STX1B, AK5, AP3B2, Flotillin1+2, GRM1, GRM2, GRM5, GLURD2, ITPR1 (EP14003703.7), ATP1A3, NBC1 (EP14003958.7), Neurochondrin (EP15001186), KCNA2, Septin 3+5+6+7+11 and Sez6L2. Preferably, the antigen is associated with a neurological autoimmune disease, preferably autoimmune encephalitis. Preferably two, three, four, five, six, seven, eight, nine, ten, fifteen, twenty or more such antigens are used in addition, more preferably NMDAR is one of them. Variants binding to an autoantibody associated with the neurological autoimmune disease may be used. These antigens are described in the state of the art, for example in Darnell/Posner, Paraneoplastic Syndromes, Oxford University Press, 2011.

In a preferred embodiment, the term “a polypeptide comprising one or more from the group comprising Gluk1, Gluk2, Gluk3, Gluk4 and Gluk5”, as used herein, comprises one or more from Gluk1, Gluk2, Gluk3, Gluk4 and Gluk5, which, in the case of two or more polypeptides, are separate polypeptides, spatially separate or in a mixture, or part of one polypeptide, wherein the two or more of Gluk1, Gluk2, Gluk3, Gluk4 and Gluk5 are part of a fusion polypeptide. A polypeptide may be designed such that it is folded or forms a folded complex together with another polypeptide, for example if both polypeptides comprise separate subunits of a receptor such as GABA A or the complex comprising flotillin1 and flotillin 2. In the case of Gluk1, Gluk2 and Gluk3, the complex may be a homomeric complex. In the case of Gluk4 or Gluk5 it comprises a) one of Gluk4 or Gluk5 and b) one of Gluk1, Gluk2 and Gluk3. A variant of each of Gluk1, Gluk2, Gluk3, Gluk4 and Gluk5 may be used in each case. In a preferred embodiment, the polypeptide comprises a Gluk1 homomer or a variant thereof. In a preferred embodiment, the polypeptide comprises a Gluk2 homomer or a variant thereof. In a preferred embodiment, the polypeptide comprises a Gluk3 homomer or a variant thereof. In a preferred embodiment, the polypeptide comprises a Gluk4 heteromer or a variant thereof. In a preferred embodiment, the polypeptide comprises a Gluk5 heteromer or a variant thereof.

In a preferred embodiment, the method according to the present invention comprises the step providing the carrier according to the present invention. A polypeptide comprising one or more from the group comprising Gluk1, Gluk2, Gluk3, Gluk4 and Gluk5, preferably Gluk2 or a variant thereof may be immobilized on a solid surface of the carrier or the means may be configured for immobilizing the polypeptide on the carrier, which may be done at a later stage, but before removing the sample and washing the carrier. The carrier may then be contacted with the sample suspected of comprising the antibody under conditions allowing for binding of any antibodies to one or more from the group comprising Gluk1, Gluk2, Gluk3, Gluk4 and Gluk5, preferably Gluk2. The sample may then be removed and the carrier may be washed to remove any remaining sample. A means for detecting the autoantibody carrying a detectable label such as a fluorescent dye may then be contacted with the carrier under conditions allowing formation of a complex between any bound autoantibody and the means. The carrier may be washed again. Finally, the presence of the autoantibody is detected by checking whether the means such as a secondary antibody may be detected. Preferably an ELISA or an immunofluorescence assay using a mammalian cell or tissue expressing a polypeptide comprising one or more from the group comprising Gluk1, Gluk2, Gluk3, Gluk4 and Gluk5, preferably Gluk2 or a variant thereof is used. In the case of immunofluorescence, a distinct pattern determined by the expression pattern of the transporter in the cell or the tissue indicates the presence of the antibody, as carried out or shown in the examples. In the case of an ELISA or another semi-quantitative or quantitative detection method a value above a cut off value determined as known in the art indicates the presence of the antibody.

A competitive assay, a capture bridge assay, an immunometric assay, a class-specific second antibody on the solid phase, a direct or indirect class capture assay may also be used. The principle of each of these formats is detailed in The Immunoassay Handbook, 3^{rd} edition, edited by David Wild, Elsevier, 2005. Briefly, in a competitive format, the antibody to be detected may compete with a recombinant antibody for binding sites on the antigen, which is one or more from the group comprising Gluk1, Gluk2, Gluk3, Gluk4 and Gluk5, preferably Gluk2 or a variant thereof. Alternatively, a sample comprising the antibody may be preincubated with the antigen, followed by exposure to immobilized antigen or antigen configured for immobilization in one reaction and may be exposed to the antigen without pre-incubation with the antigen in another reaction to show specific binding. In a capture bridge assay, two antigen molecules bind to two antigen binding sites on the (auto)antibody to be detected. One of the antigen molecules is labeled and the other one immobilized or configured for immobilization, preferably via an affinity tag and a ligand binding specifically to said affinity tag. In an immunometric assay, the antibody to be detected binds to the antigen, which is immobilized after or before the binding.

The antibody is detected using a means for detecting an antibody such as a labeled secondary antibody. In a direct class capture assay, the antibody to be detected is immobilized using a means for capturing an antibody and detected using a labeled antigen. In an indirect class capture assay, the antibody to be detected is immobilized using a means for capturing an antibody. It is detected using at least one, preferably two molecules of the antigen which bind to the antibody to be detected, and a means for detecting an antibody such as a labeled secondary antibody.

In a preferred embodiment, the carrier and a means for capturing and/or a means for detecting an antibody are configured for immobilizing the means on the solid surface of the carrier. A particularly preferred way for the configuration or the immobilization is modifying the carrier and/or the means such that they comprise an affinity tag and a ligand to the affinity tag. Alternatively, the carrier or the means may comprise reactive chemical groups such as thiol, amino, epoxide, ester and anhydride groups. In a preferred embodiment, the term “immobilized”, as used herein, refers to a molecule bound to a solid carrier insoluble in an aqueous solution, more preferably via a covalent or non-covalent bond, electrostatic interactions, encapsulation, unspecific absorption, printing or entrapment, for example by denaturing a globular polypeptide in a gel, or via hydrophobic interactions, most preferably via one or more covalent bonds. Various suitable carriers, for example paper, polystyrene, metal, silicon or glass surfaces, microfluidic channels, membranes, beads such as magnetic beads, column chromatography media, biochips, polyacrylamide gels and the like have been described in the literature, for example in Kim, D., and Herr, A. E. (2013), Protein immobilization techniques for microfluidic assays, Biomicrofluidics 7(4), 041501. This way, the immobilized molecule, together with the insoluble carrier, may be separated from an aqueous solution in a straightforward manner, for example by centrifugation or decanting. An immobilized molecule may be immobilized in a reversible or irreversible manner. For example, the immobilization is reversible if the molecule interacts with the carrier via ionic interactions that can be masked by addition of a high concentration of salt or if the molecule is bound via a cleavable covalent bond such as a disulfide bridge which may be cleaved by addition of thiol-containing reagents. By contrast, the immobilization is irreversible if the molecule is tethered to the carrier via a covalent bond that cannot be cleaved in aqueous solution, for example a bond formed by reaction of an epoxide group and an amine group as frequently used to couple lysine side chains to affinity columns. The protein may be indirectly immobilized, for example by immobilizing an antibody or other entity having affinity to the molecule, followed by formation of a complex to the effect that the molecule-antibody complex is immobilized. Various ways to immobilize molecules are described in the literature, for example in Kim, D., Herr, and A. E. (2013), Protein immobilization techniques for microfluidic assays, Biomicrofluidics 7(4), 041501. In addition, various reagents and kits for immobilization reactions are commercially available, for example from Pierce Biotechnology.

According to the present invention, a cell may be provided, which comprises one or more expression vectors comprising a nucleotide sequence encoding a polypeptide comprising one or more from the group comprising Gluk1, Gluk2, Gluk3, Gluk4 and Gluk5, preferably Gluk2 or a variant thereof under the control of a promotor, optionally a strong and/or inducible promotor. In the case of Gluk1, Gluk2 or Gluk3, the cell may comprise one vector only comprising a nucleotide sequence encoding the respective polypeptide, since homomers may be formed. In the case of Gluk4 and Gluk5, the cell comprises at least two vectors, one comprising a nucleotide sequence encoding Gluk1, Gluk2 and Gluk3 and one comprising a nucleotide sequence encoding Gluk4 or Gluk5. The vector may encode for an N-terminal or C-terminal affinity tag fused to one or more from the group comprising Gluk1, Gluk2, Gluk3, Gluk4 and Gluk5, preferably Gluk2 in a polypeptide comprising one or more from the group comprising Gluk1, Gluk2, Gluk3, Gluk4 and Gluk5, preferably Gluk2, preferably via a linker sequence. The cell may be cultured under conditions allowing for the expression of Gluk1, Gluk2, Gluk3, Gluk4 and/or Gluk5, preferably the Gluk2. Any expressed Gluk1, Gluk2, Gluk3, Gluk4 and Gluk5, preferably Gluk2 may then be purified, preferably using the affinity tag. However, a non-purified Gluk1, Gluk2, Gluk3, Gluk4 and Gluk5, preferably Gluk2 may also be used in some embodiments. Subsequently it is immobilized on the carrier according to the present invention. In a preferred embodiment, the cell, nucleic acid or vector may be used for the manufacture of a kit for the diagnosis of a neurological autoimmune disease.

According to the present invention, a medical or diagnostic device such as the diagnostically useful carrier may be prepared by expressing a recombinant polypeptide comprising a variant of one or more from the group comprising Gluk1, Gluk2, Gluk3, Gluk4 and Gluk5, preferably Gluk2 comprising an affinity tag, optionally with an artificial linker, which may include a protease cleavage site, in a cell such as a eukaryotic or prokaryotic cell. Contacting the polypeptide with a ligand binding specifically to the affinity tag, which ligand is immobilized on a solid phase, washing the solid phase such that non-specifically bound material from the cell is removed and eluting the expressed variant from the solid phase, preferably by adding an excess of non-immobilized ligand. The variant may then be immobilized on the device. Optionally, the affinity tag may be removed by contacting the variant with a protease, preferably a protease recognizing the protease cleavage site, before the immobilization. The affinity tag may be selected from the group of tags comprising His, immobilized nickel, glutathione, chitin, 18A, ACP, Aldehyde, Avi, BCCP, Calmodulin, Chitin binding protein, E-Tag, ELK16, FLAG, flash, poly glutamate, poly aspartate, GST, GFP, HA, Isope, maltose binding protein, myc, nus, NE, ProtA, ProtC, Tho1d4, S-Tag, SnoopTag, SpyTag, SofTag, Streptavidin, Strep-tag II, T7 Epitope Tag, TAP, TC, Thioredoxin, Ty, V5, VSV, biotin, Xpress Tag and a recombinant antibody binding to the ligand to an affinity tag. Useful proteases include, but are not limited to TEV, Thrombin, Factor Xa or Enteropeptidase. Suitable linkers are part of vectors, for example pET vector series (Novagen).

In a preferred embodiment, the absence or presence of two or more antibodies to two or more antigens is detected simultaneously, i.e. at the same time.

In another preferred embodiment, the presence or absence of at least one autoantibody other than an antibody binding specifically to one or more from the group comprising Gluk1, Gluk2, Gluk3, Gluk4 and Gluk5, preferably Gluk2 is detected, preferably from the group comprising an autoantibody binding specifically to NMDAR, an autoantibody binding specifically to LgI1, an autoantibody binding specifically to AMPA1, an autoantibody binding specifically to AMPA2, an autoantibody binding specifically to CASPR2, an autoantibody binding specifically to GABA B, an autoantibody binding specifically to GABA A, an autoantibody binding specifically to DPPX, an autoantibody binding specifically to IGLON5, an autoantibody binding specifically to Hu, an autoantibody binding specifically to Yo, an autoantibody binding specifically to CRMP5, an autoantibody binding specifically to Ri, an autoantibody binding specifically to Ma2, an autoantibody binding specifically to Amphiphysin, an autoantibody binding specifically to Recoverin, an autoantibody binding specifically to RGS8, an autoantibody binding specifically to DAGLA, an autoantibody binding specifically to NSF, an autoantibody binding specifically to STX1B, an autoantibody binding specifically to DNM1, an autoantibody binding specifically to VAMP2, an autoantibody binding specifically to Anna-3, an autoantibody binding specifically to Zic-4, an autoantibody binding specifically to SOX1 (U.S. Pat. No. 7,314,721), an autoantibody binding specifically to PCA2, an autoantibody binding specifically to Tr, an autoantibody binding specifically to glutamic acid decarboxylase, an autoantibody binding specifically to AK5, an autoantibody binding specifically to AP3B2, an autoantibody binding specifically to Flotillin1/2, an autoantibody binding specifically to GRM1, an autoantibody binding specifically to GRM2, an autoantibody binding specifically to GRM5, an autoantibody binding specifically to GLURD2, an autoantibody binding specifically to ITPR1, an autoantibody binding specifically to KCNA2, an autoantibody binding specifically to NCDN, an autoantibody binding specifically to Septin 3+5+6+7+11 and an autoantibody binding specifically to Sez6L2. In a preferred embodiment, the detection of the presence of any of these autoantibodies aids in the diagnosis or implies a diagnosis of a neurological autoimmune disease. In a preferred embodiment, two or more autoantibodies are detected in the same sample and preferably essentially simultaneously. The carrier according to the present invention may be configured for detecting two or more autoantibodies in the same sample, preferably essentially simultaneously. In a preferred embodiment, the carrier, polypeptide or kit according to the present invention is used to detect the presence or absence of two or more autoantibodies, among them one binding specifically to Gluk1, Gluk2, Gluk3, Gluk4 or Gluk5, preferably Gluk5, more preferably to diagnose or aid in the diagnosis of a subject or determine whether the sample is from a subject with an increased likelihood to suffer in the past, presence and/or future from a disease such as a cancer and/or a neurological autoimmune disease, preferably a neurological autoimmune disease.

In a preferred embodiment, the presence or absence of two or more antibodies is detected and can be distinguished. In other words, a signal indicating the presence of an antibody indicates which of the two antibodies is present. In a preferred embodiment, the absence or presence of two or more antibodies is detected in spatially separate reactions, more preferably in different reaction mixtures in separate vessels. In another preferred embodiment, a signal indicating the presence of one of the two autoantibodies may be distinguished from a signal indicating the presence of the other autoantibody. This may be achieved by using different detectable labels, more preferably distinguishable fluorophores. For example, a fluorophore emitting green light and another one emitting red light may be used.

In a preferred embodiment, the presence or absence of two or more antibodies is detected, but cannot be distinguished. In a preferred embodiment, their absence or presence is detected in a one pot reaction, preferably in two or more reactions in the same reaction vessel without spatial separation, and with no signal distinction. In other words, a signal indicates that at least one of the two antibodies is present, but not which one. In a preferred embodiment, two or more antigens may be present in a mixture.

In a preferred embodiment, the sample comprises a representative set of antibodies, more preferably IgG, IgA and IgM class antibodies, most preferably IgG class antibodies. It is preferably selected from the group comprising whole blood, plasma, serum, cerebrospinal fluid and saliva. The sample may be a liquid sample or a dried blood spot made using the sample, preferably whole blood, plasma, serum or capillary blood, preferably capillary blood.

In a preferred embodiment, the sample is from an organism having a brain and producing autoantibodies, preferably from the group comprising mammal and birds, more preferably a mammal from the group comprising a human, a non-human primate, a rodent, a cow, a horse, a dog, a cat, a bear, a donkey, a sheep, a goat, a camel and a dromedary, most preferably a human. In another preferred embodiment it is from a bird, more preferably from the group comprising a chicken, a parrot and a falcon. The animal may have gone through extensive training, for example for assisting people in need, for riding or for hunting.

The autoantibody to be detected or a means for detecting or capturing an antibody binds specifically to its interaction partner, which is an antigen in the case of an autoantibody or an antibody in the case of a means for detecting an antibody, preferably the autoantibody binding specifically to one or more from the group comprising Gluk1, Gluk2 and Gluk3, preferably Gluk2. In a preferred embodiment, the term “binding specifically”, as used herein, preferably means that the binding reaction is stronger than a binding reaction characterized by a dissociation constant of 1×10⁻⁵ M, more preferably 1×10⁻⁷ M, more preferably 1×10⁻⁸ M, more preferably 1×10⁻⁹ M, more preferably 1×10⁻¹⁰ M, more preferably 1×10⁻¹¹ M, more preferably 1×10⁻¹² M, as determined by surface plasmon resonance using Biacore equipment at 25° C. in PBS buffer at pH 7.

In a preferred embodiment, the term “autoantibody”, as used herein, refers to an antibody which binds specifically to a structure, preferably an antigen, from the organism which produces said antibody. The organism is preferably a patient suspected of or actually suffering from a disease, preferably a mammalian, more preferably human patient. Such an autoantibody has a constant region, as have other antibodies of the same class from the same organism. Particularly preferably, the autoantibody is a mammalian autoantibody, even more preferably a human autoantibody, even more preferably a human autoantibody of class IgG, IgM or IgA, preferably IgG. The variable domain is capable of binding specifically against the antigen. The constant domain binds specifically to molecules recognizing the constant domain of IgG class antibodies such as secondary antibodies.

According to the present invention, an antibody binding specifically to a polypeptide from the group comprising, Gluk1, Gluk2, Gluk3, Gluk4 and Gluk5, preferably Gluk2 is provided or isolated. The person skilled in the art is familiar with the isolation or purification of antibodies. Comprehensive instructions are available in the prior art, for example in Affinity Chromatography Vol. 1 Antibody by GE Healthcare, April 2016 and Thermo Scientific Pierce Antibody Production and Purification Technical Handbook, Version 2, www.thermoscientific.com. For example, specific purification steps may involve affinity chromatography using a polypeptide comprising Gluk1, Gluk2, Gluk3, Gluk4 or Gluk5, preferably Gluk2 or a variant thereof immobilized to beads by coupling using primary amines and/or Protein G.

The teachings of the present invention may not only be carried out using the polypeptides, in particular a polypeptide comprising the native sequence of a polypeptide referred to such as one or more from the group comprising Gluk1, Gluk2, Gluk3, Gluk4 and Gluk5, preferably Gluk2 or nucleic acids having the exact sequences referred to in this application explicitly, for example by function, name, sequence or accession number, or implicitly, but also using variants of such polypeptides or nucleic acids.

In a preferred embodiment, the term “variant”, as used herein, may refer to at least one fragment of the full-length sequence referred to, more specifically one or more amino acid or nucleic acid sequence which is, relative to the full-length sequence, truncated at one or both termini by one or more amino acids. Such a fragment comprises or encodes for a peptide having at least 6, 7, 8, 10, 12, 15, 20, 25, 50, 75, 100, 150, 200, 300, 400, 500, 600, 620, 640, or 660 successive amino acids of the original sequence or a variant thereof. The total length of the variant may be at least 6, 7, 8, 9, 10, 11, 12, 20, 25, 30, 40, 50, 60, 70, 80, 90, 100, 200, 300, 400, 450, 500, 520, 540, 560, 580, 600, 610, 620, 630, 640, 650, 660, 680, 700, 720, 740, 760, 780, 800, 820, 840, 860, 880 or 900 or more amino acids.

The term “variant” relates not only to at least one fragment, but also to a polypeptide or a fragment thereof comprising amino acid sequences that are at least 40, 50, 60, 70, 75, 80, 85, 90, 92, 94, 95, 96, 97, 98, 99, 99.3, 99.4, 99.5, 99.6, 99.7, 99.8 or 99.9, preferably at least 99.3% identical to the reference amino acid sequence referred to or the fragment thereof, wherein amino acids other than those essential for the biological activity, for example the ability of an antigen to bind to an (auto)antibody, or the fold or structure of the polypeptide are deleted or substituted and/or one or more such essential amino acids are replaced in a conservative manner and/or amino acids are added such that the biological activity of the polypeptide is preserved. The state of the art comprises various methods that may be used to align two given nucleic acid or amino acid sequences and to calculate the degree of identity, see for example Arthur Lesk (2008), Introduction to bioinformatics, Oxford University Press, 2008, 3^{rd} edition. In a preferred embodiment, the ClustalW software (Larkin, M. A., Blackshields, G., Brown, N. P., Chenna, R., McGettigan, P. A., McWilliam, H., Valentin, F., Wallace, I. M., Wilm, A., Lopez, R., Thompson, J. D., Gibson, T. J., Higgins, D. G. (2007): Clustal W and Clustal X, Version 2.0, Bioinformatics, 23, 2947-2948) is used using default setting. When designing variants of Gluk1, Gluk2, Gluk3, Gluk4 or Gluk5, the person skilled in the art will preferably consider that the autoantibody to be detected binds to extracellular epitopes, the location of which may be taken from data base entries. For example, extracellular domains of Gluk2 include SEQ ID NO: 6 and SEQ ID NO: 7. The person skilled in the art is aware that autoantibodies often bind best to conformational epitopes. Therefore, the use of an antigen comprising folded parts of the extracellular domain is preferred. Various publications can be used to design variants of the NMDAR. Sharma et al. disclosed that the N-terminal domain is essential for the epitope-autoantibody recognition (Sharma, R., A-Saleem, F. H., Puligedda, R. D., Rattelle, A., Lynch, D. R., and Dessain, S. K. (2018) Membrane-bound and soluble forms of an NMDA receptor extracellular domain retain epitopes targeted in auto-immune encephalitis, BMC Biotechnology 18:41). They show that is may be used in a fusion protein. It is important that at least one NR1 subunit remains that comprises amino acids 25-380 of the NR1 subunit, because the inventors of U.S. Pat. No. 7,972,976 have shown that this is the part of the antigen that is essential for recognition of the autoantibody. Further guidance based on site-directed mutagenesis may be found by Gleichman et al. (2012) who identified an epitope comprising N368 and G369 (Gleichman, A. J., Spruce, L. A., Dalmau, J., Seeholzer, S. H., and Lynch, D. R. (2012) Anti-NMDA Receptor Encephalitis Antibody binding Is Dependent on Amino Acid Identity of a Small Region within the GluN1 Amino Terminal Domain, J. Neuroscience, 32(32)11083-11094).

In a preferred embodiment, the polypeptide and variants thereof may, in addition, comprise chemical modifications, for example isotopic labels or covalent modifications such as glycosylation, phosphorylation, acetylation, decarboxylation, citrullination, methylation, hydroxylation and the like. The person skilled in the art is familiar with methods to modify polypeptides. Any modification is designed such that it does not abolish the biological activity of the variant.

Moreover, variants may also be generated by N- or/and C-terminal fusion of polypeptides, fragments or variants thereof with other known polypeptides or variants thereof or artificial sequences such as linkers and comprise active portions or domains, preferably having a sequence identity of at least 70, 75, 80, 85, 90, 92, 94, 95, 96, 97, 98 or 99% when aligned with the active portion of the reference sequence, wherein the term “active portion”, as used herein, refers to an amino acid sequence, which is less than the full length amino acid sequence or, in the case of a nucleic acid sequence, codes for less than the full length amino acid sequence, respectively, and/or is a variant of the natural sequence, but retains at least some of the biological activity. Preferably the active portion is an active portion of one or more from the group comprising Gluk1, Gluk2, Gluk3, Gluk4 and Gluk5, preferably Gluk2. The polypeptide may comprise additional sequences, preferably artificial sequences for example linkers or binding epitopes. Any fused sequences are chosen such that the ability of one or more from the group comprising Gluk1, Gluk2, Gluk3, Gluk4 and Gluk5, preferably Gluk2 or a variant thereof to bind specifically to the antibody to be detected or the diagnostic reliability, in particular sensitivity and/or specificity, is not significantly altered, let alone abolished.

In a preferred embodiment, the term “variant” of a nucleic acid comprises nucleic acids the complementary strand of which hybridizes, preferably under stringent conditions, to the reference or wild type nucleic acid. Stringency of hybridization reactions is readily determinable by one of ordinary skilled in the art, and in generally is an empirical calculation dependent on probe length, washing temperature and salt concentration. In general, longer probes require higher temperatures for proper annealing, while shorter probes less so. Hybridization generally depends on the ability of denatured DNA to reanneal to complementary strands present in an environment below their melting temperature: The higher the degree of desired homology between the probe and hybridizable sequence, the higher the relative temperature which may be used. As a result, it follows that higher relative temperatures would tend to make the reaction conditions more stringent, while lower temperature less so. For additional details and explanation of stringency of hybridization reactions, see Ausubel, F. M. (1995), Current Protocols in Molecular Biology. John Wiley & Sons, Inc. Moreover, the person skilled in the art may follow the instructions given in the manual Boehringer Mannheim GmbH (1993) The DIG System Users Guide for Filter Hybridization, Boehringer Mannheim GmbH, Mannheim, Germany and in Liebl, W., Ehrmann, M., Ludwig, W., and Schleifer, K. H. (1991) International Journal of Systematic Bacteriology 41: 255-260 on how to identify DNA sequences by means of hybridization. In a preferred embodiment, stringent conditions are applied for any hybridization, i.e. hybridization occurs only if the probe is 70% or more identical to the target sequence. Probes having a lower degree of identity with respect to the target sequence may hybridize, but such hybrids are unstable and will be removed in a washing step under stringent conditions, for example lowering the concentration of salt to 2×SSC or, optionally and subsequently, to 0.5×SSC, while the temperature is, in order of increasing preference, approximately 50° C.-68° C., approximately 52° C.-68° C., approximately 54° C.-68° C., approximately 56° C.-68° C., approximately 58° C.-68° C., approximately 60° C.-68° C., approximately 62° C.-68° C., approximately 64° C.-68° C., approximately 66° C.-68° C. In a particularly preferred embodiment, the temperature is approximately 64° C.-68° C. or approximately 66° C.-68° C. It is possible to adjust the concentration of salt to 0.2×SSC or even 0.1×SSC. Nucleic acid sequences having a degree of identity with respect to the reference or wild type sequence of at least 70, 80, 90, 91, 92, 93, 94, 95, 96, 97, 98, 99% may be isolated. In a preferred embodiment, the term variant of a nucleic acid sequence, as used herein, refers to any nucleic acid sequence that encodes the same amino acid sequence, preferably one or more from the group comprising Gluk1, Gluk2, Gluk3, Gluk4 and Gluk5, preferably Gluk2 and a variant thereof, as the reference nucleic acid sequence, in line with the degeneracy of the genetic code.

In a preferred embodiment, the term “sensitivity” refers to the number of samples correctly determined as positive relative to the total number of samples examined. In a preferred embodiment, the term “specificity” refers to number of samples correctly determined as negative relative to the total number of samples examined.

The variant of the polypeptide, more specifically one or more from the group comprising Gluk1, Gluk2 and Gluk3, preferably Gluk2 has biological activity. In a preferred embodiment, such biological activity is the ability to bind specifically to an autoantibody binding specifically to one or more from the group comprising Gluk1, Gluk2, Gluk3, Gluk4 and Gluk5, preferably Gluk2, or another antigen such as NMDAR, as found in a patient suffering from a neurological autoimmune disease associated with the presence of such autoantibody in a sample, preferably autoimmune encephalitis. For example, whether or not a variant of the polypeptide has such biological activity may be checked by determining whether or not it binds specifically to an autoantibody from a sample of such a patient comprising an autoantibody binding specifically to wild type antigen, preferably as determined by indirect immunofluorescence as described in the experimental section of this application. In a preferred embodiment, the isoform of Gluk1 represented by Uniprot data base code P39086-2 is a variant of Gluk1. In a preferred embodiment, isoforms of Gluk2 including those represented by Uniprot data base codes Q13002-2, Q13002-3, Q13002-4, Q13002-5, Q13002-6 and Q13002-7 are variants of Gluk2. In a preferred embodiment, the isoform of Gluk3 represented by Uniprot data base code Q13003-2 is a variant of Gluk3. In s preferred embodiment, the isoform of Gluk5 represented by Uniprot data base code Q16478-2 is variant of Gluk5.

According to the present invention, a cell is provided which overexpresses a polypeptide comprising one or more from the group comprising Gluk1, Gluk2, Gluk3, Gluk4 and Gluk5, preferably Gluk2 or a variant thereof, preferably in combination with at least one additional cell which overexpresses another antigen from the group comprising Hu, Yo, Ri, CV2, PNMA1, PNMA2, DNER/Tr, ARHGAP26, ITPR1 (EP14003703.7), ATP1A3, NBC1 (EP14003958.7), Neurochondrin (EP15001186), CARPVIII, Zic4, SOX1 (U.S. Pat. No. 7,314,721), Ma, MAG, MP0, MBP, GAD65, amphiphysin, recoverin, GABA A receptor, GABA B receptor, glycine receptor, gephyrin, IgLON5, DPPX, aquaporin-4, MOG, NMDA receptor, AMPA receptors, GRM1, GRM5, LGI1, VGCC, mGluR1, CASPR2, ATP1A3, also referred to as alpha 3 subunit of human neuronal Na(+)/K(+) ATPase (EP14171561.5) and Flotillin1/2 (EP3101424) a variant thereof comprised in a polypeptide, preferably NMDA receptor (NMDAR).

In a preferred embodiment, the term “overexpressing”, as used herein, means that the cell has been transfected with a nucleic acid that comprises a nucleic acid sequence encoding a polypeptide comprising one or more from the group comprising Gluk1, Gluk2, Gluk3, Gluk4 and Gluk5, preferably Gluk2 or the other antigen or a variant thereof under the control of a promotor. Consequently, the transfected cell expresses more polypeptide recognized by the autoantibody binding specifically to be detected than the same type of cell normally would, probably at least 10, 20, 30, 50, 100, 200 or 500% more as judged by quantitative Western Blot. The promotor may be an inducible promotor, which allows for the induction of expression by addition of an inducer. The person skilled in the art is familiar with protocols and vectors for transiently overexpressing a polypeptide in a eukaryotic cell, for example the pTriEx system from Novagen and with protocols and vectors for stably transfecting a eukaryotic cell, for example the pcDNA™4/TO vector system from Invitrogen.

In a preferred embodiment, one or more fixed mammalian cells may be used. In a preferred embodiment, the term “fixed” cell, as used herein, refers to a cell that has been treated with a reactive chemical compound to the effect that the cell is no longer metabolically active, but still presents its epitopes for immunostaining with antibodies and their subsequent detection, for example by fluorescence. More preferably, the reactive chemical compound is selected from the group comprising acetone, formalin, methanol and ethanol or mixtures thereof, preferably all of them. The person skilled in the art is familiar with protocols that may be used to prepare fixed cells.

According to the present invention, the cell is or the cells are on a carrier for microscopic immunofluorescence analysis. Such a carrier may be a glass slide. The cell on the glass slide may be covered with a mounting buffer. A mounting medium is a liquid which helps maintain a near physiological pH to maintain the molecular structure of any diagnostically relevant molecular and their epitopes, is compatible with the emission of a fluorescence signal and prevents a premature loss of fluorescence due to bleaching of the fluorophore. It may be selected from the group comprising water, glycerol, natural oil or plastic or a mixture thereof, preferably water and glycerol. Various compositions are described in the state of the art, for example in “Mountants and Antifades”, published by Wright Cell Imaging Facility, Toronto Western Research Institute University Health Network, (https://de.scribd.com/document/47879592/Mountants-Antifades), Krenek et al. (1989) J. Immunol. Meth 117, 91-97 and Nairn et al. (1969) Clin. Exp. Immunol. 4, 697-705.

A cover glass may be placed on top of the composition comprising the sample and the mounting medium. Slides with cover glasses (FB 112d-1005-1 or ZZ 3000-0112) are available from EUROIMMUN Medizinische Labordiagnostika, AG. However, any carrier compatible with microscopic analysis of the fluorescence pattern may be used. The carrier may comprise a mock-transfected cell, which has been transfected with the same vector as the cell overexpressing a polypeptide comprising one or more from the group comprising Gluk1, Gluk2, Gluk3, Gluk4 and Gluk5, preferably Gluk2 or a variant thereof, but without the nucleic acid encoding for the latter. Such mock-transfected cell may serve as a negative control. The carrier is configured for analysis using an immunofluorescence microscope.

In a preferred embodiment, the carrier may comprise a field comprising the cell according to the invention. In addition, the carrier may comprise additional fields. The fields are preferably surrounded by a hydrophobic surface. Each of these fields may comprise a cell overexpressing another antigen or a variant thereof. A field may comprise a section of primate cerebellum, rat cerebellum or rat hippocampus and thalamus. In a preferred embodiment, the cell is a eukaryotic cell overexpressing the polypeptide, such as a cell selected from the group comprising HEK, Hela, CHO and Jurkat cells and derivatives thereof. In a preferred embodiment, the cell is a recombinant cell overexpressing the polypeptide, which is preferably under the control of a heterologous strong promoter.

In another preferred embodiment, the diagnostically useful carrier is a bead. Various beads for numerous applications are commercially available, mainly based on carbohydrate, for example Sepharose or agarose, or plastic. They may contain active or activatable chemical groups such as a carboxyl or tosyl or ester group, which can be utilized for the immobilization of a means for specifically capturing an antibody. Preferably, the beads are beads having an average diameter of from 0.1 μm to 10 μm, from 0.5 μm to 8 μm, from 0.75 μm to 7 μm or from 1 μm to 6 μm. Preferably, the bead is provided in the form of an aqueous suspension having a bead content of from 10 to 90%, preferably from 20 to 80%, preferably from 30 to 70%, more preferably from 40 to 60% (w/w). The person skilled in the art is familiar with such beads (Diamandis, E. P., Christopoulos. T. K., Immunoassays, 1996, Academic Press), which are commercially available, for example Bio-Plex COOH beads MC10026-01 or 171-506011 from Bio-Rad. Preferably, the carrier comprises at least two beads, one comprising Gluk1, Gluk2, Gluk3, Gluk4 or Gluk5, preferably Gluk2, or a variant thereof, and one comprises another antigen.

In another preferred embodiment, the carrier is a microtiter plate comprising at least 8 wells that may be used for ELISA. At least one of the wells is coated with the means for specifically capturing an antibody, either directly or indirectly, preferably one or more from the group comprising Gluk1, Gluk2, Gluk3, Gluk4 and Gluk5, preferably Gluk2 or a variant thereof. At least 3, preferably 4, more preferably 5 calibrators, at defined concentrations may be used to set up a calibration curve for semi-quantitative analysis. When the inventive method is carried out, the calibrators, which typically cover a range of concentrations covering the calibrating curve, may be processed and developed in parallel to the samples. A secondary antibody comprising a detectable label such as an enzymatically active label may be provided, for example a label having horse radish peroxidase activity or alkaline phosphatase activity or an enzyme capable of chemiluminescence.

In another preferred embodiment, the carrier is a test strip, preferably a blot, more preferably a line blot (Raoult, D., and Dasch, G. A. (1989), The line blot: an immunoassay for monoclonal and other antibodies. Its application to the serotyping of gram-negative bacteria. J. Immunol. Methods, 125 (1-2), 57-65; WO2013041540). One or more from the group comprising Gluk1, Gluk2, Gluk3, Gluk4 and Gluk5, preferably Gluk2, or a variant thereof is immobilized in the form of a band or dot on the test strip. In a preferred embodiment, one or more additional antigen is immobilized on it, preferably from the group comprising NMDAR, LgI1, AMPA1, AMPA2, CASPR2, GABA B, GABA A, DPPX, IGLON5, Hu, Yo, CV2/CRMP5, Ri, Ma1, Ma2, Amphiphysin, Recoverin, RGS8, DAGLA, STX1B, AK5, AP3B2, Flotillin1+2, GRM1, GRM2, GRM5, GLURD2, ITPR1, KCNA2, NCDN, SOX1, TR(DNER), Zic4, Septin 3+5+6+7+11 and Sez6L2, more preferably NMDAR or a variant thereof. If two or more antigens are used, they are preferably spatially separated on the carrier. Preferably, the width of the bands is at least 30, more preferably 40, 50, 60, 70 or 80% the width of the test strip. The test strip may comprise one or more control bands, preferably for confirming that it has been contacted with sample sufficiently long and under adequate conditions, preferably with serum or CSF. The test strip may comprise one or more control bands for confirming that it has been contacted with all necessary reagents for developing bands or with a secondary antibody, preferably a labeled secondary antibody. The test strip may comprise a negative control band. The test strip may comprise a positive control band. The test strip may comprise a cut-off control band indicating the minimum staining that qualifies as an indication that an autoantibody has been detected. The test strip may comprise two or more calibrator bands. Various blots are described in the art, for example in van Oss, J. C., and Regenmortel, M. H. V., Immunochemistry, 1994, Marcell Dekker, in particular Chapter 35.

In another preferred embodiment, the carrier is a microarray. In a preferred embodiment, the term “microarray”, as used herein, refers to a chip spotted with a variety of spatially separate antigens, preferably at least 5, more preferably 10, 20, 30, 40, 50, 80 or 100. Preferably each antigen is a peptide comprising or consisting of 5 to 25, preferably 7 to 15 successive amino acids spanning a fragment of a mammalian. At least one antigen is a polypeptide comprising one or more from the group comprising Gluk1, Gluk2, Gluk3, Gluk4 and Gluk5, preferably Gluk2 or a variant thereof. Preferably two or more antigens are a polypeptide comprising one or more from the group comprising Gluk1, Gluk2, Gluk3, Gluk4 and Gluk5, preferably Gluk2 or a variant thereof. A secondary antibody comprising a label, preferably a fluorescent label, may be used for the detection. Preferably at least one additional antigen or a variant thereof is spotted.

According to the present invention, a means for detecting an antibody or an antibody is used, which is a molecule binding specifically to the antibody and allowing detection, typically comprising a detectable label. In a preferred embodiment, a detectable label may be used to distinguish a population of molecules from others using biophysical detection methods. It is preferably selected from the group comprising a fluorescent, a radioactive, a chemiluminescent label, a heavy metal such as gold label, a nanoparticle, a bead or an enzymatically active label, preferably one catalyzing a colorimetric reaction. In a preferred embodiment, a fluorescent label is selected from the group comprising Alexa dyes, FITC, TRITC and green fluorescent protein (GFP). Iodine-125 may be used as radioactive label. In a preferred embodiment, an enzymatically active label is selected from the group comprising horseradish peroxidase, glucose oxidase, beta galactosidase, alkaline phosphatase and luciferase. In a preferred embodiment, a chemiluminescent label is selected from the group comprising luminol or a derivative, an acridinium ester and luciferase. The person skilled in the art is able to choose suitable labels and to attach them to proteins, nucleic acids and other molecules (Hassanzadeh L, Chen S, Veedu R N. Radiolabeling of Nucleic Acid Aptamers for Highly Sensitive Disease-Specific Molecular Imaging. Pharmaceuticals (Basel). 2018; 11(4):106. Published 2018 Oct. 15. doi: 10.3390/ph11040106 Hassanzadeh L, Chen S, Veedu R N. Radiolabeling of Nucleic Acid Aptamers for Highly Sensitive Disease-Specific Molecular Imaging. Pharmaceuticals (Basel). 2018; 11(4):106. Published 2018 Oct. 15. doi:10.3390/ph11040106, Bioconjugate Techniques, 3rd Edition (2013) by Greg T. Hermanson, Obermaier C, Griebel A, Westermeier R. Principles of protein labeling techniques. Methods Mol Biol. 2015; 1295: 153-65), and a wide range of labeled molecules are commercially available. According to the present invention, a means for capturing an antibody such as an IgG class antibody is a molecule binding specifically to the antibody to be immobilized and capable of immobilizing it, either because it is immobilized itself or configured for immobilization, preferably via an affinity tag. It may be a secondary antibody binding to a class of antibodies, preferably IgG class antibodies, more preferably human IgG class antibodies, or an aptamer or a protein such as protein G. The means for detecting a captured antibody is a ligand binding specifically to one or more from the group comprising Gluk1, Gluk2, Gluk3, Gluk4 and Gluk5, preferably Gluk2 or another antigen. The means for detecting an immobilized antibody is a ligand binding specifically to the class of the antibody to be detected and may be selected from the group comprising a secondary antibody, and a ligand binding specifically, Protein G or variant thereof or an aptamer or an antibody binding specifically to the immobilized antibody.

In accordance with the present invention, the term “secondary antibody” in its broadest sense is to be understood to refer to any kind of “binding moiety”, preferably binding protein, capable of specific binding to an IgA, IgG and/or IgM class antibody or a fragment thereof such as a constant domain of a particular Ig class of a selected species, preferably human species. Non-limiting examples of binding moieties include antibodies, for example antibodies immunologically or genetically derived from any species, for example human, chicken, camel, llama, lamprey, shark, goat, rodent, cow, dog, rabbit, etc., antibody fragments, domains or parts thereof, for example Fab, Fab′, F(ab′)₂, scFab, Fv, scFv, VH, VHH, VL, VLRs, and the like, diabodies, monoclonal antibodies (mAbs), polyclonal antibodies (pAbs), mAbdAbs, phage display-derived binders, affibodies, heteroconjugate antibodies, bispecific antibodies, evibodies, lipocalins, anticalins, affibodies, avimers, maxibodies, heat shock proteins such as GroEL and GroES, trans-bodies, DARPins, aptamers, C-type lectin domains such as tetranectins; human γ-crystallin and human ubiquitin-derived binders such as affilins, PDZ domain-derived binders; scorpion toxin and/or Kunitz-type domain binders, fibronectin-derived binders such as adnectins, receptors, ligands, lectins, streptavidin, biotin, including derivatives and/or combinations thereof such as bi-/multi-specific formats formed from two or more of these binding molecules. Various antibody-derived and alternative (i.e. non-antibody) binding protein scaffolds including methods of generation thereof are known in the art (e.g. reviewed in Chiu M L et al., Antibodies (Basel), (2019); 8(4):55; Simeon R. & Chen Z., Protein Cell. (2018); 9(1):3-14; and Chapter 7—Non-Antibody Scaffolds from Handbook of Therapeutic Antibodies (2007) edited by Stefan Dübel, U.S. Pat. No. 7,166,697, Rothe C & Skerra A., BioDrugs. (2018); 32(3):233-243; Gebauer M & Skerra A, Curr Opin Biotechnol. (2019); 60:230-241; Feldwisch, J & Tolmachev, V. (2012) Methods Mol. Biol. 899:103-126; Wikman M et al., Protein Eng Des Sel. (2004); 17(5):455-62; Silverman J et al. (2005), Nat Biotechnol 23:1558-1561; Plückthun A., Annu Rev Pharmacol Toxicol. (2015); 55:489-511; Hosse R J et al. (2006). Protein Sci 15:14-27; Hackel B J, et al. (2008) J Mol Biol 381:1238-1252. In a preferred embodiment, a secondary antibody is an antibody binding to all antibodies from an antibody or immunoglobulin class, preferably a human antibody class, preferably IgA and/or IgG and/or IgM antibodies, preferably IgG. Secondary antibodies may recognize the constant domain of said class or one or more epitopes across the sequence or 3D structure shared by antibodies of the Ig class of interest. Secondary antibodies are typically from a mammal other than a human or from a bird, preferably from chicken, rabbit, mouse, rat, horse, pig, donkey, goat, cow, camel, llama, or non-human primate. A secondary antibody may be a monoclonal, preferably recombinant antibody or may be a polyclonal antibody. A wide range of them is commercially available.

In a preferred embodiment, a ligand to an affinity tag, as used herein, is an artificial entity binding specifically to an affinity tag, typically a chemically synthesized modification or a recombinant protein or peptide attached to a molecule of interest. The ligand to an affinity tag depends on the type of affinity tag chosen and may be selected from the group comprising His, immobilized nickel, glutathione, chitin, 18A, ACP, Aldehyde, Avi, BCCP, Calmodulin, Chitin binding protein, E-Tag, ELK16, FLAG, flash, poly glutamate, poly aspartate, GST, GFP, HA, Isope, maltose binding protein, myc, nus, NE, ProtA, ProtC, Tho1d4, S-Tag, SnoopTag, SpyTag, SofTag, Streptavidin, Strep-tag II, T7 Epitope Tag, TAP, TC, Thioredoxin, Ty, V5, VSV, biotin, Xpress Tag and a recombinant antibody binding to the ligand to an affinity tag.

According to the present invention, an autoantibody binding specifically to one or more from the group comprising Gluk1, Gluk2, Gluk3, Gluk4 and Gluk5, preferably Gluk2 is detected or provided, preferably in a solution comprising one or more, more preferably all from the group comprising an artificial buffer, a preservative, a stabilizer and an artificial anticoagulant. An artificial buffer is a buffer which is synthetic and/or may not occur in the body of the patient or at least at concentrations well below the concentration used. The buffer may be selected from the group comprising Tris, phosphate, Tricine, acetate, MOPS, MES, carbonate, citrate and HEPES. In a preferred embodiment, the term “preservative” as used herein, refers to a substance inhibiting microbial growth and/or chemical degradation in a liquid solution and may preferably be selected from the group comprising acid, lactic acid, nitrate, nitrite, antibiotics, a protease inhibitor and ethanol. In a preferred embodiment, the term “stabilizer” refers to a substance that will prevent or decrease the unspecific absorption of protein by the reaction vessel. It is preferably a non-human protein, more preferably bovine serum albumin. In a preferred embodiment, the autoantibody is an autoantibody binding specifically to a Gluk1 homomer. In a preferred embodiment, the autoantibody is an autoantibody binding specifically to a Gluk2 homomer. In a preferred embodiment, the autoantibody is an autoantibody binding specifically to a Gluk3 homomer. In a preferred embodiment, the autoantibody is an autoantibody binding specifically to a heteromer comprising Gluk4 and at least one from Gluk1, Gluk2 and Gluk3. In a preferred embodiment, the autoantibody is an autoantibody binding specifically to a heteromer comprising Gluk5 and at least one from Gluk1, Gluk2 and Gluk3.

Various methods or uses according to the invention can be conducted with a sample from a subject as described herein. These methods or uses can also be characterized as “in vitro” methods or “in vitro” uses.

In a preferred embodiment, the term “chemical solution reactive with a detectable label” refers to a compound in a liquid which, upon exposure to the detectable label, emits a detectable signal. The solution may comprise a chromogenic substrate of an enzymatically active label. For example, 3,3′,5,5′ tetramethylbenzidine/H₂O₂ may be used if the label is a peroxidase. The solution may comprise a small inorganic or organic compound capable of reacting with a chemiluminescent label. In the case of an acridinium ester, a mixture of H₂O₂ and sodium hydroxide is frequently used as the chemical solution. Various other chemical solutions and detectable labels are known in the art (Weeks, I., Beheshti, I., McCapra, F., Campbell, A. K., Woodhead, J. S. (1983): Acridinium esters as high specific activity labels in immunoassay, Clin Chem, Volume 29, Page 1474-1479), Thermo Scientific Pierce Antibody Production and Purification Technical Handbook, Version 2, www.thermoscientific.com).

In a preferred embodiment, the term “diagnosis”, as used herein, is to be used in its broadest possible sense and may to any kind of procedure aiming to obtain information instrumental in the assessment whether a patient, known or an anonymous subject from a cohort, suffers or is likely or more likely than the average or a comparative subject, the latter preferably having similar symptoms, to suffer from certain a disease or disorder in the past, at the time of the diagnosis or in the future, to find out how the disease is progressing or is likely to progress in the future or to evaluate the responsiveness of a patient or patients in general with regard to a certain treatment, for example the administration of immunosuppressive drugs, or to find out whether a sample is from such a patient. Such information may be used for a clinical diagnosis but may also be obtained by an experimental and/or research laboratory for the purpose of general research, for example to determine the proportion of subjects suffering from the disease in a patient cohort or in a population. In other words, the term “diagnosis” comprises not only diagnosing, but also prognosticating and/or monitoring the course of a disease or disorder, including monitoring the response of one or more patients to the administration of a drug or candidate drug, for example to determine its efficacy. While the result may be assigned to a specific patient for clinical diagnostic applications and may be communicated to a medical doctor or institution treating said patient, this is not necessarily the case for other applications, for example in diagnostics for research purposes, where it may be sufficient to assign the results to a sample from an anonymized patient. In another preferred embodiment, the detection of an autoantibody binding specifically to one or more from the group comprising Gluk1, Gluk2, Gluk3, Gluk4 and Gluk5, preferably Gluk2 is considered to imply a definitive diagnosis of a neurological autoimmune disease because of the presence of the autoantibody.

In a preferred embodiment, the methods and products according to the present invention may be used for interaction studies, including determining whether a drug candidate or other compound may interfere with the binding of an autoantibody binding specifically to one or more from the group comprising Gluk1, Gluk2, Gluk3, Gluk4 and Gluk5, preferably Gluk2 or may affect any downstream process or the strength of its binding to its target. In preferred embodiment, they may be used for monitoring the immune response, more preferably the emergence and/or titer of antibodies to one or more from the group comprising Gluk1, Gluk2, Gluk3, Gluk4 and Gluk5, preferably Gluk2, following the administration of an immunogenic composition comprising a polypeptide comprising one or more from the group comprising Gluk1, Gluk2, Gluk3, Gluk4 and Gluk5, preferably Gluk2 or an immunogenic variant thereof, for example to a mammal, which may be a mammal other than a human such as a laboratory animal.

In a preferred embodiment, the methods and products may be used for providing reagents such as an antibody to one or more from the group comprising Gluk1, Gluk2, Gluk3, Gluk4 and Gluk5, preferably Gluk2 which may serve as a positive control or a calibrator for a diagnostic test or for developing and/or validating a diagnostic test. In a preferred embodiment, the term “validating”, as used herein, refers to a procedure for establishing an assay in a specific environment based on a previously known principle and confirming that it yields useful results. For example, while this application discloses the usefulness of an antibody to one or more from the group comprising Gluk1, Gluk2, Gluk3, Gluk4 and Gluk5, preferably Gluk2 as a marker for a diagnosis, a clinical or research laboratory may need to confirm the diagnostic value of the results before routinely using the test for their patients or a new group of patients, for example a group of animals, which may not have been known previously to suffer from a disease or condition.

In another preferred embodiment, the methods and products according to the present invention may be used for determining the concentration of an antibody binding specifically to one or more from the group comprising Gluk1, Gluk2, Gluk3, Gluk4 and Gluk5, preferably Gluk2, preferably Gluk2. In a more preferred embodiment, said antibody is an autoantibody from a patient suffering from a neurological autoimmune disease. In another preferred embodiment, said antibody is a recombinant antibody which binds to one or more from the group comprising Gluk1, Gluk2, Gluk3, Gluk4 and Gluk5, preferably Gluk2, but is recognized by a secondary antibody binding specifically to human IgG class antibodies, preferably IgG1, IgG2, IgG3 and IgG4 isotypes. In a more preferred embodiment, such a concentration needs to be determined for the purposes of research, for the preparation or for monitoring the quality of reagents, animal models or devices that may or may not be used for the diagnosis of a neurological autoimmune disease.

In many cases the mere detection of the autoantibody, in other words determining whether or not detectable levels of the antibody are present in the sample, is sufficient for the diagnosis. In a more preferred embodiment, this may involve determining whether the concentration is at least 10%, preferably 20%, 50%, 100%, 200%, 500%, 1,000%, 2,000%, 2,500%, 5,000%, 1,0000%, 2,0000%, 5,0000%, 100,000%, 1,000,000% or 10,000,000% times higher than the concentration of the antibody of interest found in the average healthy subject. If the autoantibody can be detected, this will be information instrumental for the clinician's diagnosis. It may indicate an increased likelihood that the patient suffers from a disease.

The person skilled in the art will appreciate that a clinician does usually not arrive at the conclusion whether or not the patient suffers or is likely to suffer from a disease, condition or disorders solely on the basis of a single diagnostic parameter, but needs to take into account other aspects, for example the presence of other autoantibodies, markers, blood parameters, clinical assessment of the patient's symptoms or the results of medical imaging or other non-invasive methods such as polysomnography, to arrive at a conclusive diagnosis. See Baenkler H. W. (2012), General aspects of autoimmune diagnostics, in Renz, H., Autoimmune diagnostics, 2012, de Gruyter, page 3. The value of a diagnostic agent or method may also reside the possibility to rule out one disease, thus allowing for the indirect diagnosis of another. In a preferred embodiment, the meaning of any symptoms or diseases referred to throughout this application is in line with the person skilled in the art's understanding as of the filing date or, preferably, earliest priority date of this application as evidenced by textbooks and scientific publications. In a preferred embodiment, the inventive methods or uses or products are not used, taken alone, to arrive at a definite, final diagnosis.

In a preferred embodiment, any information or data demonstrating the presence of absence of the autoantibody may be communicated to the patient or a medical doctor treating the patient orally, preferably by telephone, in a written form, preferably fax or letter, or in an electronic form via fax or via the internet, for example as an email or text message.

The inventive teachings may also be used in a method for preventing or treating a disease, preferably after a diagnosis according to the present invention, comprising the steps a) reducing the concentration of autoantibodies binding to the inventive polypeptide in the subject's blood and/or b) administering one or more immunosuppressive pharmaceutical substances, preferably selected from the group comprising rituximab, prednisone, methylprednisolone, cyclophosphamide, mycophenolate mofetil, intravenous immunoglobulin, tacrolimus, cyclosporine, methotrexate and azathioprine.

According to the present invention, the presence of an antibody may be determined in a qualitative or a quantitative manner. In a preferred embodiment, the term “detecting in a quantitative manner”, as used herein, means that not only the presence of an antibody is detected, but that a result is obtained that includes information regarding the absolute or relative amount of the antibody in the sample. In a more preferred embodiment, a value representing an absolute concentration is obtained. In another more preferred embodiment, a value representing a relative concentration or change of concentration is obtained. In another preferred embodiment, also referred to as “semi-quantitative” approach, the concentration of the antibody is placed in one of several groups, most preferably a concentration window meaning that it is virtually absent, a concentration window meaning that a borderline result is obtained and a concentration window meaning that the antibody is present. A further distinction into categories such as “weak positive” or “strong positive” signal is possible.

In a preferred embodiment, the term “autoantibody”, as used herein, refers to an antibody binding specifically to an endogenous molecule of the animal, preferably mammal, more preferably human, which produces said autoantibody, wherein the level of such antibody is more preferably elevated compared to the average healthy subject. The autoantibody may have the sequence of an antibody's constant regions from the animal, preferably human, making it, but the variable region is able to bind specifically to the endogenous molecule of the animal, more specifically one or more from the group comprising Gluk1, Gluk2, Gluk3, Gluk4 and Gluk5, preferably Gluk2. In a preferred embodiment, the autoantibody is isolated, enriched and/or purified from a sample, preferably tissue, serum, plasma, blood or CSF from the animal, preferably human. The autoantibody may be in a sample, which is preferably the type of sample to be analyzed, more preferably serum or CSF. The autoantibody can be isolated as a mixture of polyclonal, native antibodies from the animal or patient. It is not a synthetic, monoclonal or recombinant antibody. A recombinant antibody binding specifically to one or more from the group comprising Gluk1, Gluk2, Gluk3, Gluk4 and Gluk5, preferably Gluk2 may be generated using standard methods. It may be useful as a positive control and for detection assays, for example sandwich assays or competitive assays.

The method according to the present invention is preferably an in vitro method.

According to the present invention, a polypeptide, preferably the polypeptide comprising one or more from the group comprising Gluk1, Gluk2, Gluk3, Gluk4 and Gluk5, preferably Gluk2 or a variant thereof, may be a recombinant protein. In a preferred embodiment, the term “recombinant”, as used herein, refers to a polypeptide produced using genetic engineering approaches at any stage of the production process, for example by fusing a nucleic acid encoding the polypeptide to a strong promoter for overexpression in cells or tissues or by engineering the sequence of the polypeptide itself. The person skilled in the art is familiar with methods for engineering nucleic acids and polypeptides encoded (for example, described in Sambrook, J., Fritsch, E. F. and Maniatis, T. (1989), Molecular Cloning, CSH or in Brown T. A. (1986), Gene Cloning—an introduction, Chapman & Hall) and for producing and purifying native or recombinant polypeptides (for example Handbooks “Strategies for Protein Purification”, “Antibody Purification”, published by GE Healthcare Life Sciences, and in Burgess, R. R., Deutscher, M. P. (2009): Guide to Protein Purification). In another preferred embodiment, a polypeptide provided or used according to the present invention such as a polypeptide comprising a mammalian GLUK2 or a variant thereof or an antibody is an isolated polypeptide, wherein the term “isolated” means that the polypeptide has been enriched compared to its state upon production using a biotechnological or synthetic approach and is preferably pure, i.e. at least 60, 70, 80, 90, 95 or 99 percent of the polypeptide in the respective liquid consists of said polypeptide as judged by SDS polyacrylamide gel electrophoresis followed by Coomassie blue staining and visual inspection. Preferably any polypeptide on a carrier used as a means to capture an antibody is pure.

In a preferred embodiment, patients having autoantibodies to one or more from the group comprising Gluk1, Gluk2, Gluk3, Gluk4 and Gluk5, preferably Gluk2, have an increased risk of suffering from a variety of cancers comprising leukemia, graft versus host disease and non-Hodgkin lymphoma. In a preferred embodiment, the term “cancer”, as used herein, refers to is a group of diseases involving abnormal cell growth with the potential to invade or spread to other parts of the body.

In a preferred embodiment, the term “non-Hodgkin lymphoma”, as used herein, refers to a group of blood cancers that includes all types of lymphomas except Hodgkin lymphomas.

Additional background and definitions related to cancers and their diagnoses or differential diagnoses including the detection of autoantibodies may be taken from neurology textbooks available at the earliest priority date or filing date of this publication such as Kaye, Textbook of Medical Oncology, 3rd edition, Taylor & Francis: Shoenfeld, Meroni and Gershwin, Autoantibodies 3rd edition, Elsevier, in particular Part 11 including Chapter 76; and Darnell and Posner, Paraneoplastic Syndromes, Oxford University Press, 2011.

In a preferred embodiment, patients having autoantibodies to one or more from the group comprising Gluk1, Gluk2, Gluk3, Gluk4 and Gluk5, preferably Gluk2, compared to a subject without such autoantibodies, have an increased risk of suffering from a neurological autoimmune disease, preferably from the group comprising PNS, cerebellar ataxia, gait ataxia, polyneuropathy, encephalitis, preferably limbic encephalitis, epilepsy, dementia, cerebellar syndrome and hypersensitive encephalopathy and from a cancer, preferably from the group comprising as leukemia, graft versus host disease and non-Hodgkin lymphoma. In a preferred embodiment, the methods, uses and products according to the present invention may be used to diagnosis or aid in the diagnosis of to identify a person with an increased risk of suffering from such a neurological autoimmune disease and/or such a cancer.

In a preferred embodiment, the term “ataxia”, as used herein, refers to lack of voluntary coordination of muscle movements that can include gait abnormality, speech changes, and abnormalities in eye movements. Ataxia is a clinical manifestation indicating dysfunction of the parts of the nervous system that coordinate movement, such as the cerebellum.

In a preferred embodiment, the term “polyneuropathy”, as used herein, refers to a disease affecting peripheral nerves (peripheral neuropathy) in roughly the same areas on both sides of the body, featuring weakness, numbness, and burning pain.

In a preferred embodiment, the term “encephalopathy”, as used herein, refers to an altered mental state characterized by impairment of the cognition, attention, orientation, sleep-wake cycle and consciousness

In a preferred embodiment, the term “Encephalitis”, as used herein, refers to an inflammation of the brain, with symptoms including reduced or alternation in consciousness, personality changes, psychotic delusions, rigidity, headache, fever, confusion, a stiff neck, and vomiting. Complications may include seizures, hallucinations, trouble speaking, memory problems, and problems with hearing. The disease may be the result of an infection or may be an autoimmune disease, hence the detection of an autoantibody according to the invention may be used to distinguish these two types of encephalitis.

In a preferred embodiment, the term “cerebellar syndrome”, as used herein, refers to an impaired cerebellar function typically associated with ataxia, nystagmus and dysarthria.

In a preferred embodiment, the term “Paraneoplastic Neurological Syndrome” (PNS), as used herein, refers to neurological syndromes associated with the presence of a cancer associated with tumors which presents an antigen which is normally exclusive to the nervous system. As a result, an autoantibody binding specifically to the neurological antigen is produced which may then damage the nervous system. Manifestations of PNS include, but are not limited to encephalitis, typically associated with seizures, psychiatric manifestations such as hallucinations, anxiety and depression, cerebellar symptoms, such as ataxia, nystagmus and dysarthria, opsoclonus-myoclonus and sensory neuronopathy; and Lambert-Eaton myasthenic syndrome. It should be mentioned that PNS-associated tumors are often small, grow slowly and may not yet be detectable when neurological syndromes surface. PNS may be associated with one or more antibodies, which may bind specifically to an antigen from the group comprising NMDAR, LgI1, AMPA1, AMPA2, CASPR2, GABA B, GABA A, DPPX, IGLON5, Hu, Yo, CRMP5, Ri, Ma2, Amphiphysin, Recoverin, RGS8, DAGLA, NSF, STX1B, DNM1 and VAMP2, Hu, Ri, Ma, Anna-3, Zic-4, SOX1, Yo, PCA2, Tr and glutamic acid decarboxylase.

Additional background and definitions related to neurological syndromes and symptoms and their diagnoses or differential diagnoses including the detection of autoantibodies may be taken from neurology textbooks available at the earliest priority date or filing date of this publication such as Simon, Greenberg, Aminoff, Clinical Neurology, 7th edition, 2009, McGraw; Shoenfeld, Meroni and Gershwin, Autoantibodies 3rd edition, Elsevier, in particular Part 11 including Chapter 76; and Darnell and Posner, Paraneoplastic Syndromes, Oxford University Press, 2011.

As part of a diagnosis relating to the neurological syndromes associated with an autoantibody associated with the presence of one or more from the group comprising Gluk1, Gluk2, Gluk3, Gluk4 and Gluk5, preferably Gluk2, the clinician will initially consider a range of tests and risk factors which may point them either to autoimmune disease or infectious disease for many of the conditions associated with the presence of an autoantibody binding specifically to Gluk1, Gluk2, Gluk3, Gluk4 and Gluk5, preferably Gluk2 (Lancaster, J Clin Neurol. 2016 January; 12(1) https://doi.org/10.3988/jcn.2016.12.1.1, Lee and Lee, The Laboratory Diagnosis of Autoimmune Encephalitis, Journal of Epilepsy Research 6 (2), 45), preferably encephalitis. Detection of an autoantibody binding specifically to one or more from the group comprising Gluk1, Gluk2, Gluk3, Gluk4 and Gluk5, preferably Gluk2 will then confirm an autoimmune background, while the absence of an autoantibody will tempt the clinician to consider autoimmune disease associated with other autoantibodies. However, typically the presence or absence of a range of autoantibodies will then be detected, and a negative result will be a pointer to infectious diseases.

According to the present invention, a kit is provided, comprising the cell or the carrier and further comprising one or more, preferably all reagents from the group comprising a secondary antibody, preferably labeled with a detectable label, a washing solution, a positive control, a negative control, a detergent, a cover glass, a mounting medium and a physiological salt solution, preferably PBS, or salt required to prepare it. In a preferred embodiment, the positive control is a diluted sample, preferably serum or CSF, from a patient suffering from a neurological autoimmune disease or a monoclonal antibody binding specifically to one or more from the group comprising Gluk1, Gluk2, Gluk3, Gluk4 and Gluk5, preferably Gluk2. The negative control may be a diluted sample from a healthy subject, for example a blood donor. The kit may comprise instructions how to carry out the assay. Preferably, the secondary antibody is a secondary antibody binding specifically to IgG class antibodies, preferably human IgG class antibodies.

In a preferred embodiment, the present invention provides a use of the cell, the polypeptide, the carrier for the manufacture of kit a composition for the diagnosis of a disease.

In a preferred embodiment, any method or use according to the present invention may be intended for a non-diagnostic use, i.e. determining the presence of an autoantibody binding specifically to binding to one or more from the group comprising Gluk1, Gluk2, Gluk3, Gluk4 and Gluk5, preferably Gluk2, for a use other than diagnosing a patient. For example, the method or use may be for testing in vitro the efficiency of a medical device designed to remove an autoantibody from a patient's blood, wherein the testing is performed on a liquid other than patient's blood. After the use of the medical device with a patient, its capacity to remove autoantibody may be checked by running a solution comprising antibody binding specifically to one or more from the group comprising Gluk1, Gluk2, Gluk3, Gluk4 and Gluk5, preferably Gluk2 through the device, followed by use of the method according to the present invention to confirm that less or no antibody is in the solution that has been passed through the device, i.e. showing that the device has still the capacity to remove antibody from the solution.

According to the present invention, the method may be used for testing the efficacy of a drug candidate which may be used to treat patients suffering from or likely to suffer from a neurological autoimmune disease. Such a drug candidate may be any molecule capable of interfering with the interaction between one or more from the group comprising Gluk1, Gluk2, Gluk3, Gluk4 and Gluk5, preferably Gluk2 and the autoantibody.

In another preferred embodiment, the method may be for confirming the reliability of a diagnostic assay and may involve detecting an antibody binding specifically to one or more from the group comprising Gluk1, Gluk2, Gluk3, Gluk4 and Gluk5, preferably Gluk2 in a solution, which is not a sample from a patient who requires a diagnosis, but is known to comprise an antibody binding specifically to one or more from the group comprising Gluk1, Gluk2, Gluk3, Gluk4 and Gluk5, preferably Gluk2, preferably at a known concentration. For example, it may be a recombinant antibody or a sample diluted in a dilution buffer such as PBS from an anonymous patient whose identity cannot be traced back. Alternatively, the solution may be a negative control not comprising the antibody binding specifically to check the background. Such method may be run in parallel with, after or before a diagnostic method. In a preferred embodiment, any method or use according to the present invention may be intended for generating an autoantibody profile, preferably for detecting a disease in a mammal, preferably a human.

In a preferred embodiment, any method or use according to the present invention may be for identifying a subject at risk of suffering from or developing a disease and/or a tumor.

In a preferred embodiment, the method may be for detecting an antibody, preferably autoantibody binding specifically to one or more from the group comprising Gluk1, Gluk2, Gluk3, Gluk4 and Gluk5, preferably Gluk2 in a solution which is not a sample from a mammal to be diagnosed or for the purpose of providing a diagnosis. In a preferred embodiment, the problem underlying the present invention is solved by a method comprising the step contacting a device comprising a solid phase on which a polypeptide comprising one or more from the group comprising Gluk1, Gluk2, Gluk3, Gluk4 and Gluk5, preferably Gluk2 or a variant thereof is immobilized with a buffered solution comprising an antibody binding specifically to one or more from the group comprising Gluk1, Gluk2, Gluk3, Gluk4 and Gluk5, preferably Gluk2, which solution is preferably not a sample from a patient in need of a diagnosis, wherein preferably
a) the concentration of the antibody in the solution is known, and/or
b) the antibody is a recombinant antibody and/or
c) the medical or diagnostic device is contacted with two or more solutions comprising the antibody, wherein the two or more solutions have a different concentration of the antibody, and/or
d) the antibody is recognized by secondary antibodies binding specifically to IgG antibodies,

followed by detection of a signal which indicates whether or not the antibody has bound to the polypeptide, optionally a signal relating to the concentration of the antibody in the solution or solutions.

In a preferred embodiment, the present invention provides an apparatus for analyzing a sample from a patient to detect an autoantibody against one or more from the group comprising Gluk1, Gluk2, Gluk3, Gluk4 and Gluk5, preferably Gluk2, indicating an increased likelihood of a neurological autoimmune disease or of developing it, comprising:
a) a carrier, which contains a means for capturing the autoantibody from the sample when the sample is contacted with the carrier, wherein the means is the cell and the carrier is the carrier according to the present invention,
b) a detectable means capable of binding to the antibody captured by the carrier when the detectable means is contacted with the carrier, wherein the detectable means is preferably a labeled secondary antibody capable of binding to the autoantibody captured on the carrier,
c) optionally a means for removing any sample from the carrier and the detectable means, preferably by washing;
d) a detecting device for detecting the presence of the detectable means and converting the results into an electrical signal, for example a fluorescence reader or a fluorescence microscope connected with a software capable of recognizing a pattern characteristic of a stained cell overexpressing a polypeptide comprising one or more from the group comprising Gluk1, Gluk2, Gluk3, Gluk4 and Gluk5, preferably Gluk2 or a variant thereof in an image of the cell taken by the fluorescence reader or camera, and

optionally a means for receiving the electronical signal from the detecting device and determining if the level of the signal is indicative of an increased likelihood of having or developing a disease, by comparing with the patterns characteristic of wild type or non-stained cells, preferably by a mock-transfected cell or cells not positively stained by an autoantibody binding specifically to one or more from the group comprising Gluk1, Gluk2, Gluk3, Gluk4 and Gluk5, preferably Gluk2 on the same carrier, or an input reference value obtained with samples from healthy subjects or by comparing the level of signal obtained with one sample with the level of signal obtained with a second sample obtained at a later time point, preferably at least one month later.

According to the present invention, a device for removing an autoantibody binding specifically to one or more from the group comprising Gluk1, Gluk2, Gluk3, Gluk4 and Gluk5, preferably Gluk2 from blood, preferably serum of a patient suffering from a neurological autoimmune disease, wherein the device comprises a carrier with a solid phase on which a polypeptide comprising one or more from the group comprising Gluk1, Gluk2, Gluk3, Gluk4 and Gluk5, preferably Gluk2 or a variant thereof is immobilized is provided as is an ex vivo method for removing an autoantibody binding specifically to one or more from the group comprising Gluk1, Gluk2, Gluk3, Gluk4 and Gluk5, preferably Gluk2 from a patient. A device on which a polypeptide comprising one or more from the group comprising Gluk1, Gluk2, Gluk3, Gluk4 and Gluk5, preferably Gluk2 or a variant thereof or a secondary antibody or protein capturing all IgG class antibodies, among them IgG class autoantibodies to one or more from the group comprising Gluk1, Gluk2, Gluk3, Gluk4 and Gluk5Gluk3, preferably Gluk2, may be used. Suitable methods are described in Eisei Noiri and Noria Hanafusa, The Concise Manual of Apheresis Therapy, Springer Tokyo, 2014. Hamilton, P., Kanigicheria, D., Hanumapura, P., Walz, L., Kramer, D., Fischer, M., Brenchley, P., and Mitra, S. (2018) J. Clin. Aph. 33(3), 283-290. Another method is disclosed in EP3477300.

The present invention comprises a range of polypeptide sequences, more specifically

(Gluk1, as in P39086 from Uniprot))
SEQ ID NO: 1
MEHGTLLAQPGLWTRDTSWALLYFLCYILPQTAPQVLRIGGIFETVENE
PVNVEELAFKFAVTSINRNRTLMPNTTLTYDIQRINLFDSFEASRRACD
QLALGVAALFGPSHSSSVSAVQSICNALEVPHIQTRWKHPSVDNKDLFY
INLYPDYAAISRAILDLVLYYNWKTVTVYEDSTGLIRLQELIKAPSRYN
IKIKIRQLPSGNKDAKPLLKEMKKGKEFYVIFDCSHETAAEILKQILFM
GMMTEYYHYFFTTLDLFALDLELYRYSGVNMTGFRLLNIDNPHVSSIIE
KWSMERLQAPPRPETGLLDGMMTTEAALMYDAVYMVAIASHRASQLTVS
SLQCHRHKPWRLGPRFMNLIKEARWDGLTGHITFNKTNGLRKDFDLDII
SLKEEGTEKAAGEVSKHLYKVWKKIGIWNSNSGLNMTDSNKDKSSNITD
SLANRTLIVTTILEEPYVMYRKSDKPLYGNDRFEGYCLDLLKELSNILG
FIYDVKLVPDGKYGAQNDKGEWNGMVKELIDHRADLAVAPLTITYVREK
VIDFSKPFMTLGISILYRKPNGTNPGVFSFLNPLSPDIWMYVLLACLGV
SCVLFVIARFTPYEWYNPHPCNPDSDVVENNFTLLNSFWFGVGALMQQG
SELMPKALSTRIVGGIWWFFTLIIISSYTANLAAFLTVERMESPIDSAD
DLAKQTKIEYGAVRDGSTMTFFKKSKISTYEKMWAFMSSRQQTALVRNS
DEGIQRVLTTDYALLMESTSIEYVTQRNCNLTQIGGLIDSKGYGVGTPI
GSPYRDKITIAILQLQEEGKLHMMKEKWWRGNGCPEEDNKEASALGVEN
IGGIFIVLAAGLVLSVFVAIGEFIYKSRKNNDIEQAFCFFYGLQCKQTH
PTNSTSGTTLSTDLECGKLIREERGIRKQSSVHTV
(Gluk2, as in Q13002 from Uniprot)
SEQ ID NO: 2
MKIIFPILSNPVFRRTVKLLLCLLWIGYSQGTTHVLRFGGIFEYVESGP
MGAEELAFRFAVNTINRNRTLLPNTTLTYDTQKINLYDSFEASKKACDQ
LSLGVAAIFGPSHSSSANAVQSICNALGVPHIQTRWKHQVSDNKDSFYV
SLYPDFSSLSRAILDLVQFFKWKTVTVVYDDSTGLIRLQELIKAPSRYN
LRLKIRQLPADTKDAKPLLKEMKRGKEFHVIFDCSHEMAAGILKQALAM
GMMTEYYHYIFTTLDLFALDVEPYRYSGVNMTGFRILNTENTQVSSIIE
KWSMERLQAPPKPDSGLLDGFMTTDAALMYDAVHVSVAVQQFPQMTVSS
LQCNRHKPWRFGTRFMSLIKEAHWEGLTGRITFNKTNGLRTDFDLDVIS
LKEEGLEKIGTWDPASGLNMTESQKGKPANITDSLSNRSLIVTTILEEP
YVLFKKSDKPLYGNDRFEGYCIDLLRELSTILGFTYEIRLVEDGKYGAQ
DDANGQWNGMVRELIDHKADLAVAPLAITYVREKVIDFSKPFMTLGISI
LYRKPNGTNPGVFSFLNPLSPDIWMYILLAYLGVSCVLFVIARFSPYEW
YNPHPCNPDSDVVENNFTLLNSFWFGVGALMQQGSELMPKALSTRIVGG
IWWFFTLIIISSYTANLAAFLTVERMESPIDSADDLAKQTKIEYGAVED
GATMTFFKKSKISTYQKMWAFMSSRRQSVLVKSNEEGIQRVLTSDYAFL
MESTTIEFVTQRNCNLTQIGGLIDSKGYGVGTPMGSPYRDKITIAILQL
QEEGKLHMMKEKWWRGNGCPEEESKEASALGVQNIGGIFIVLAAGLVLS
VFVAVGEFLYKSKKNAQLEKRSFCSAMVEELRMSLKCQRRLKHKPQAPV
IVKTEEVINMHTFNDRRLPGKETMA
(Gluk3, as in Q13003 from Uniprot)
SEQ ID NO: 3
MTAPWRRLRSLVWEYWAGLLVCAFWIPDSRGMPHVIRIGGIFEYADGPN
AQVMNAEEHAFRFSANIINRNRTLLPNTTLTYDIQRIHFHDSFEATKKA
CDQLALGVVAIFGPSQGSCTNAVQSICNALEVPHIQLRWKHHPLDNKDT
FYVNLYPDYASLSHAILDLVQYLKWRSATWYDDSTGLLRLQELIMAPSR
YNIRLKIRQLPIDSDDSRPLLKEMKRGREFRIIFDCSHTMAAQILKQAM
AMGMMTEYYHFIFTTLDLYALDLEPYRYSGVNLTGFRILNVDNPHVSAI
VEKWSMERLQAAPRSESGLLDGVMMTDAALLYDAVHIVSVCYQRAPQMT
VNSLQCHRHKAWRFGGRFMNFIKEAQWEGLTGRIVFNKTSGLRTDFDLD
IISLKEDGLEKVGVWSPADGLNITEVAKGRGPNVTDSLTNRSLIVTTVL
EEPFVMFRKSDRTLYGNDRFEGYCIDLLKELAHILGFSYEIRLVEDGKY
GAQDDKGQWNGMVKELIDHKADLAVAPLTITHVREKAIDFSKPFMTLGV
SILYRKPNGTNPSVFSFLNPLSPDIWMYVLLAYLGVSCVLFVIARFSPY
EWYDAHPCNPGSEWENNFTLLNSFWFGMGSLMQQGSELMPKALSTRIIG
GIWWFFTLIIISSYTANLAAFLTVERMESPIDSADDLAKQTKIEYGAVK
DGATMTFFKKSKISTFEKMWAFMSSKPSALVKNNEEGIQRALTADYALL
MESTTIEYVTQRNCNLTQIGGLIDSKGYGIGTPMGSPYRDKITIAILQL
QEEDKLHIMKEKWWRGSGCPEEENKEASALGIQKIGGIFIVLAAGLVLS
VLVAVGEFVYKLRKTAEREQRSFCSTVADEIRFSLTCQRRVKHKPQPPM
MVKTDAVINMHTFNDRRLPGKDSMACSTSLAPVFP
(Gluk4, as in NP_055434.2 from NCBI)
SEQ ID NO: 4
MPRVSAPLVLLPAWLVMVACSPHSLRIAAILDDPMECSRGERLSITLAK
NRINRAPERLGKAKVEVDIFELLRDSEYETAETMCQILPKGWAVLGPSS
SPASSSIISNICGEKEVPHFKVAPEEFVKFQFQRFTTLNLHPSNTDISV
AVAGILNFFNCTTACLICAKAECLLNLEKLLRQFLISKDTLSVRMLDDT
RDPTPLLKEIRDDKTATIIIHANASMSHTILLKAAELGMVSAYYTYIFT
NLEFSLQRMDSLVDDRVNILGFSIFNQSHAFFQEFAQSLNQSWQENCDH
VPFTGPALSSALLFDAVYAWTAVQELNRSQEIGVKPLSCGSAQIWQHGT
SLMNYLRMVELEGLTGHIEFNSKGQRSNYALKILQFTRNGFRQIGQWHV
AEGLSMDSHLYASNISDTLFNTTLWVTTILENPYLMLKGNHQEMEGNDR
YEGFCVDMLKELAEILRFNYKIRLVGDGVYGVPEANGTWTGMVGELIAR
KADLAVAGLTITAEREKVIDFSKPFMTLGISILYRVHMGRKPGYFSFLD
PFSPGVWLFMLLAYLAVSCVLFLVARLTPYEWYSPHPCAQGRCNLLVNQ
YSLGNSLWFPVGGFMQQGSTIAPRALSTRCVSGVWWAFTLIIISSYTAN
LAAFLTVQRMDVPIESVDDLADQTAIEYGTIHGGSSMTFFQNSRYQTYQ
RMWNYMYSKQPSVFVKSTEEGIARVLNSNYAFLLESTMNEYYRQRNCNL
TQIGGLLDTKGYGIGMPVGSVFRDEFDLAILQLQENNRLEILKRKWWEG
GKCPKEEDHRAKGLGMENIGGIFWLICGLIVAIFMAMLEFLWTLRHSEA
TEVSVCQEMVTELRSIILCQDSIHPRRRRAAVPPPRPPIPEERRPRGTA
TLSNGKLCGAGEPDQLAQRLAQEAALVARGCTHIRVCPECRRFQGLRAR
PSPARSEESLEWEKTTNSSEPE
(Gluk5, as in NP_002079.3 from NCBI)
SEQ ID NO: 5
MPAELLLLLIVAFASPSCQVLSSLRMAAILDDQTVCGRGERLALALARE
QINGIIEVPAKARVEVDIFELQRDSQYETTDTMCQILPKGWSVLGPSSS
PASASTVSHICGEKEIPHIKVGPEETPRLQYLRFASVSLYPSNEDVSLA
VSRILKSFNYPSASLICAKAECLLRLEELVRGFLISKETLSVRMLDDSR
DPTPLLKEIRDDKVSTIIIDANASISHLILRKASELGMTSAFYKYILTT
MDFPILHLDGIVEDSSNILGFSMFNTSHPFYPEFVRSLNMSWRENCEAS
TYLGPALSAALMFDAVHVWSAVRELNRSQEIGVKPLACTSANIWPHGTS
LMNYLRMVEYDGLTGRVEFNSKGQRTNYTLRILEKSRQGHREIGVWYSN
RTLAMNATTLDINLSQTLANKTLVVTTILENPYVMRRPNFQALSGNERF
EGFCVDMLRELAELLRFRYRLRLVEDGLYGAPEPNGSWTGMVGELINRK
ADLAVAAFTITAEREKVIDFSKPFMTLGISILYRVHMGRKPGYFSFLDP
FSPAVWLFMLLAYLAVSCVLFLAARLSPYEWYNPHPCLRARPHILENQY
TLGNSLWFPVGGFMQQGSEIMPRALSTRCVSGVWWAFTLIIISSYTANL
AAFLTVQRMEVPVESADDLADQTNIEYGTIHAGSTMTFFQNSRYQTYQR
MWNYMQSKQPSVFVKSTEEGIARVLNSRYAFLLESTMNEYHRRLNCNLT
QIGGLLDTKGYGIGMPLGSPFRDEITLAILQLQENNRLEILKRKWWEGG
RCPKEEDHRAKGLGMENIGGIFIVLICGLIIAVFVAVMEFIWSTRRSAE
SEEVSVCQEMLQELRHAVSCRKTSRSRRRRRPGGPSRALLSLRAVREMR
LSNGKLYSAGAGGDAGSAHGGPQRLLDDPGPPSGARPAAPTPCTHVRVC
QECRRIQALRASGAGAPPRGLGVPAEATSPPRPRPGPAGPRELAEHE
(sequence of extracellular domain from Gluk2):
SEQ ID NO: 6
TTHVLRFGGIFEYVESGPMGAEELAFRFAVNTINRNRTLLPNTTLTYDT
QKINLYDSFEASKKACDQLSLGVAAIFGPSHSSSANAVQSICNALGVPH
IQTRWKHQVSDNKDSFYVSLYPDFSSLSRAILDLVQFFKWKTVTVYDDS
TGLIRLQELIKAPSRYNLRLKIRQLPADTKDAKPLLKEMKRGKEFHVIF
DCSHEMAAGILKQALAMGMMTEYYHYIFTTLDLFALDVEPYRYSGVNMT
GFRILNTENTQVSSIIEKWSMERLQAPPKPDSGLLDGFMTTDAALMYDA
VHWSVAVQQFPQMTVSSLQCNRHKPWRFGTRFMSLIKEAHWEGLTGRIT
FNKTNGLRTDFDLDVISLKEEGLEKIGTWDPASGLNMTESQKGKPANIT
DSLSNRSLIVTTILEEPYVLFKKSDKPLYGNDRFEGYCIDLLRELSTIL
GFTYEIRLVEDGKYGAQDDANGQWNGMVRELIDHKADLAVAPLAITYVR
EKVIDFSKPFMTLGISILYRKPNGTNPGVFSFLNPLSP
(sequence of extracellular domain from Gluk2)
SEQ ID NO: 7
AFLTVERMESPIDSADDLAKQTKIEYGAVEDGATMTFFKKSKISTYDKM
WAFMSSRRQSVLVKSNEEGIQRVLTSDYAFLMESTTIEFVTQRNCNLTQ
IGGLIDSKGYGVGTPMGSPYRDKITIALLQLQEEGKLHMMKEKWWRGNG
CPEEESKEASALGVQN
(peptide identified by mass spec)
SEQ ID NO: 8
MESPIDSADDLAK
SEQ ID NO: 9
MSTMHLLTFALLFSCSFARAACDPKIVNIGAVLSTRKHEQMFREAVNQA
NKRHGSWKIQLNATSVTHKPNAIQMALSVCEDLISSQVYAILVSHPPTP
NDHFTPTPVSYTAGFYRIPVLGLTTRMSIYSDKSIHLSFLRTVPPYSHQ
SSVWFEMMRVYNWNHIILLVSDDHEGRAAQKRLETLLEERESKAEKVLQ
FDPGTKNVTALLMEARELEARVIILSASEDDAATVYRAAAMLNMTGSGY
VWLVGEREISGNALRYAPDGIIGLQLINGKNESAHISDAVGVVAQAVHE
LLEKENITDPPRGCVGNTNIWKTGPLFKRVLMSSKYADGVTGRVEFNED
GDRKFANYSIMNLQNRKLVQVGIYNGTHVIPNDRKIIWPGGETEKPRGY
QMSTRLKIVTIHQEPFVYVKPTMSDGTCKEEFTVNGDPVKKVICTGPND
TSPGSPRHTVPQCCYGFCIDLLIKLARTMNFTYEVHLVADGKFGTQERV
NNSNKKEWNGMMGELLSGQADMIVAPLTINNERAQYIEFSKPFKYQGLT
ILVKKEIPRSTLDSFMQPFQSTLWLLVGLSVHWAVMLYLLDRFSPFGRF
KVNSEEEEEDALTLSSAMWFSWGVLLNSGIGEGAPRSFSARILGMVWAG
FAMIIVASYTANLAAFLVLDRPEERITGINDPRLRNPSDKFIYATVKQS
SVDIYFRRQVELSTMYRHMEKHNYESAAEAIQAVRDNKLHAFIWDSAVL
EFEASQKCDLVTTGELFFRSGFGIGMRKDSPWKQNVSLSILKSHENGFM
EDLDKTWWRYQECDSRSNAPATLTFENMAGVFMLVAGGIVAGIFLIFIE
IAYKRHKDARRKQMQLAFAAVNVWRKNLQDRKSGRAEPDPKKKATFRAI
TSTLASSFKRRRSSKDTSTGGGRGALQNQKDTVLPRRAIEREEGQLQLC
SRHRES
SEQ ID NO: 10
MESERSKRMGNACIPLKRIAYFLCLLSALLLTEGKKPAKPKCPAVCTCT
KDNALCENARSIPRTVPPDVISLSFVRSGFTEISEGSFLFTPSLQLLLF
TSNSFDVISDDAFIGLPHLEYLFIENNNIKSSSRHTFRGLKSLIHLSLA
NNNLQTLPKDIFKGLDSLTNVDLRGNSFNCDCKLKWLVEWLGHTNATVE
DSYCEGPPEYKKRKINSLSSKDFDCHTEFAKSQDLPYQSLSSDTFSYLN
DEYWIAQPFTGKCIFLEWDHVEKTFRNYDNITGTSTWCKPMETQLYWAQ
LFGGSHIYKRDSFANKFIKIQDIEILKIRKPNDIETFKIENNWYFWADS
SKAGFTTIYKWNGNGFYSHQSLHAWYRDTDVEYLEIVRTPQTLRTPHLI
LSSSSQRPVSYQWNKATQLFTNQTDIPNMEDVYAVKHFSVKGDVYICLT
RFIGDSKVMKWGGSSFQDSQRMPSRGSMVFQPLQINNYQYAILGSDYSF
TQVYNWDAEKAKFVKFQELNVQAPRSFTHVSINKRNFLFASSFKGNTQI
YKHVIVDLSA
SEQ ID NO: 11
MLLLLLLAPLFLRPPGAGGAQTPNATSEGCQIIHPPWEGGIRYRGLTRD
QVKAINFLPVDYEIEYVCRGEREWGPKVRKCLANGSWTDMDTPSRCVRI
CSKSYLTLENGKVFLTGGDLPALDGARVDFRCDPDFHLVGSSRSICSQG
QWSTPKPHCQVNRTPHSERRAVYIGALFPMSGGWPGGQACQPAVEMALE
DVNSRRDILPDYELKLIHHDSKCDPGQATKYLYELLYNDPIKIILMPGC
SSVSTLVAEAARMWNUVLSYGSSSPALSNRQRFPTFFRTHPSATLHNPT
RVKLFEKWGWKKIATIQQTTEVFTSTLDDLEERVKEAGIEITFRQSFFS
DPAVPVKNLKRQDARIIVGLFYETEARKVFCEVYKERLFGKKYVWFLSG
WYADNWFKIYDPSINCTVDEMTEAVEGHITTEIVMLNPANTRSISNMTS
QEFVEKLTKRLKRHPEETGGFQEAPLAYDAIWALALALNKTSGGGGRSG
VRLEDFNYNNQTITDQIYRAMNSSSFEGVSGHWFDASGSRMAWTLSEQL
QGGSYKKIGYYDSTKDDLSWSKTDKWIGGSPPADQTLVIKTFRFLSQKL
FISVSVLSSLGIVLAWCLSFNIYNSHVRYIQNSQPNLNNLTAVGCSLAL
AAVFPLGLDGYHIGRNQFPFVCQARLWLLGLGFSLGYGSMFTKIVWWHT
VFTKKEEKKEWRKTLEPWKLYATVGLLVGMDVLTLAIWQIVDPLHRTIE
TFAKEEPKEDIDVSILPQLEHCSSRKMNTWLGIFYGYKGLLLLLGIFLA
YETKSVSTEKINDHRAVGMAIYNVAVLCLITAPVTMILSSQQDAAFAFA
SLAIVFSSYITLWVLFVPKMRRLITRGEWQSEAQDTMKTGSSTNNNEEE
KSRLLEKENRELEKIIAEKEERVSELRHQLQSRQQLRSRRHPPTPPEPS
GGLPRGPPEPPDRLSCDGSRVHLLYK
SEQ ID NO: 12
MRKSPGLSDCLWAWILLLSTLTGRSYGQPSLQDELKDNTTVFTRILDRL
LDGYDNRLRPGLGERVTEVKTDIFVTSFGPVSDHDMEYTIDVFFRQSWK
DERLKFKGPMTVLRLNNLMASKIWTPDTFFHNGKKSVAHNMTMPNKLLR
ITEDGTLLYTMRLTVRAECPMHLEDFPMDAHACPLKFGSYAYTRAEVVY
EWTREPARSVWAEDGSRLNQYDLLGQTVDSGIVQSSTGEYVVMTTHFHL
KRKIGYFVIQTYLPCIMTVILSQVSFWLNRESVPARTVFGVTTVLTMTT
LSISARNSLPKVAYATAMDWFIAVCYAFVFSALIEFATVNYFTKRGYAW
DGKSWPEKPKKVKDPLIKKNNTYAPTATSYTPNLARGDPGLATIAKSAT
IEPKEVKPETKPPEPKKTFNSVSKIDRLSRIAFPLLFGIFNLVYWATYL
NREPQLKAPTPHQ
SEQ ID NO: 13
MWGLAGGRLFGIFSAPVLVAWCCAQSVNDPGNMSFVKETVDKLLKGYDI
RLRPDFGGPPVCVGMNIDIASIDMVSEVNMDYTLTMYFQQYWRDKRLAY
SGIPLNLTLDNRVADQLWWPDTYFLNDKKSFVHGVTVKNRMIRLHPDGT
VLYGLRITTTAACMMDLRRYPLDEQNCTLEIESYGYTTDDIEFYWRGGD
KAVTGVERIELPQFSIVEHRLVSRNVVFATGAYPRLSLSFRLKRNIGYF
ILQTYMPSILITILSWWSFWINYDASAARVALGITTVLTMTTINTHLRE
TLPKIPYVKAIDMYLMGCFVFVFLALLEYAFVNYIFFGRGPQRQKKLAE
KTAKAKNDRSKSESNRVDAHGNILLTSLEVHNEMNEVSGGIGDTRNSAI
SFDNSGIQYRKQSMPREGHGRFLGDRSLPHKKTHLRRRSSQLKIKIPDL
TDVNAIDRWSRIVFPFTFSLFNLVYWLYYVN
SEQ ID NO: 14
MSSPNIWSTGSSVYSTPVFSQKMTVWILLLLSLYPGFTSQKSDDDYEDY
ASNKTWVLTPKVPEGDVTVILNNLLEGYDNKLRPDIGVKPTLIHTDMYV
NSIGPVNAINMEYTIDIFFAQTWYDRRLKFNSTIKVLRLNSNMVGKIWI
PDTFFRNSKKADAHWITTPNRMLRIWNDGRVLYTLRLTIDAECQLQLHN
FPMDEHSCPLEFSSYGYPREEIVYQWKRSSVEVGDTRSWRLYQFSFVGL
RNTTEWKTTSGDYWMSVYFDLSRRMGYFTIQTYIPCTLIWLSWVSFWIN
KDAVPARTSLGITTVLTMTTLSTIARKSLPKVSYVTAMDLFVSVCFIFV
FSALVEYGTLRYFVSNRKPSKDKDKKKKNPLLRMFSFKAPTIDIRPRSA
TIQMNNATHLQERDEEYGYECLDGKDCASFFCCFEDCRTGAWRHGRIHI
RIAKMDSYARIFFPTAFCLFNLVYWVSYLYL
SEQ ID NO: 15
MSDKMSSFLHIGDICSLYAEGSTNGFISTLGLVDDRCWQPETGDLNNPP
KKFRDCLFKLCPMNRYSAQKQFWKAAKPGANSTTDAVLLNKLHHAADLE
KKQNETENRKLLGTVIQYGNVIQLLHLKSNKYLTVNKRLPALLEKNAMR
VTLDEAGNEGSWFYIQPFYKLRSIGDSWIGDKVLNPVNAGQPLHASSHQ
LVDNPGCNEVNSVNCNTSWKIVLFMKWSDNKDDILKGGDWRLFHAEQEK
FLTCDEHRKKQHVFLRTTGRQSATSATSSKALWEVEWQHDPCRGGAGYW
NSLFRFKHLATGHYLAAEVDPDFEEECLEFQPSVDPDQDASRSRLRNAQ
EKMVYSLVSVPEGNDISSIFELDPTTLRGGDSLVPRNSYVRLRHLCTNT
WWHSTNIPIDKEEEKPVMLKIGTSPVKEDKEAFAIVPVSPAEVRDLDFA
NDASKVLGSIAGKLEKGTITQNERRSVTKLLEDLVYFVTGGTNSGQDVL
EVVFSKPNRERQKLMREQNILKQIFKLLQAPFTDCGDGPMLRLEELGDQ
RHAPFRHICRLCYRVLRHSQQDYRKNQEYIAKQFGFMQKQIGYDVLAED
TITALLHNNRKLLEKHITAAEIDTFVSLVRKNREPRFLDYLSDLCVSMN
KSIPVTQELICKAVLNPTNADILIETKLVLSRFEFEGVSSTGENALEAG
EDEEEVWLFWRDSNKEIRSKSVRELAQDAKEGQKEDRDVLSYYRYQLNL
FARMCLDRQYLAINEISGQLDVDLILRCMSDENLPYDLRASFCRLMLHM
HVDRDPQEQVTPVKYARLWSEIPSEIAIDDYDSSGASKDEIKERFAQTM
EFVEEYLRDWCQRFPFSDKEKNKLTFEVVNLARNLIYFGFYNFSDLLRL
TKILLAILDCVHVTTIFPISKMAKGEENKGNNDVEKLKSSNVMRSIHGV
GELMTQVVLRGGGFLPMTPMAAAPEGNVKQAEPEKEDIMVMDTKLKIIE
ILQFILNVRLDYRISCLLCIFKREFDESNSQTSETSSGNSSQEGPSNVP
GALDFEHIEEQAEGIFGGSEENTPLDLDDHGGRTFLRVLLHLTMHDYPP
LVSGALQLLFRHFSQRQEVLQAFKQVQLLVTSQDVDNYKQIKQDLDQLR
SIVEKSELWYKGQGPDETMDGASGENEHKKTEEGNNKPQKHESTSSYNY
RVVKEILIRLSKLCVQESASVRKSRKQQQRLLRNMGAHAWLELLQIPYE
KAEDTKMQEIMRLAHEFLQNFCAGNQQNQALLHKHINLFLNPGILEAVT
MQHIFMNNFQLCSEINERWQHFVHCIETHGRNVQYIKFLQTIVKAEGKF
IKKCQDMVMAELVNSGEDVLVFYNDRASFQTLIQMMRSERDRMDENSPL
MYHIHLVELLAVCTEGKNVYTEIKCNSLLPLDDIVRWTHEDCIPEVKIA
YINFLNHCYVDTEVEMKEIYTSNHMWKLFENFLVDICRACNNTSDRKHA
DSILEKYVTEIVMSIVTTFFSSPFSDQSTTLQTRQPVFVQLLQGVFRVY
HCNWLMPSQKASVESCIRVLSDVAKSRAIAIPVDLDSQVNNLFLKSHSI
VQKTAMNWRLSARNAARRDSVLAASRDYRNIIERLQDIVSALEDRLRPL
VQAELSVLVDVLHRPELLFPENTDARRKCESGGFICKLIKHTKQLLEEN
EEKLCIKVLQTLREMMTKDRGYGEKLISIDELDNAELPPAPDSENATEE
LEPSPPLRQLEDHKRGEALRQVLVNRYYGNVRPSGRRESLTSFGNGPLS
AGGPGKPGGGGGGSGSSSMSRGEMSLAEVQCHLDKEGASNLVIDLIMNA
SSDRVFHESILLAIALLEGGNTTIQHSFFCRLTEDKKSEKFFKVFYDRM
KVAQQEIKATVTVNTSDLGNKKKDDEVDRDAPSRKKAKEPTTQITEEVR
DQLLEASAATRKAFTTFRREADPDDHYQPGEGTQATADKAKDDLEMSAV
ITIMQPILRFLQLLCENHNRDLQNFLRCQNNKTNYNLVCETLQFLDCIC
GSTTGGLGLLGLYINEKNVALINQTLESLTEYCQGPCHENQNCIATHES
NGIDIITALILNDINPLGKKRMDLVLELKNNASKLLLAIMESRHDSENA
ERILYNMRPKELVEVIKKAYMQGEVEFEDGENGEDGAASPRNVGHNIYI
LAHQLARHNKELQSMLKPGGQVDGDEALEFYAKHTAQIEIVRLDRTMEQ
IVFPVPSICEFLTKESKLRIYYTTERDEQGSKINDFFLRSEDLFNEMNW
QKKLRAQPVLYWCARNMSFWSSISFNLAVLMNLLVAFFYPFKGVRGGTL
EPHWSGLLWTAMLISLAIVIALPKPHGIRALIASTILRLIFSVGLQPTL
FLLGAFNVCNKIIFLMSFVGNCGTFTRGYRAMVLDVEFLYHLLYLVICA
MGLFVHEFFYSLLLFDLVYREETLLNVIKSVTRNGRSIILTAVLALILV
YLFSIGYLFFKDDFILEVDRLPNETAVPETGESLASEFLFSDVCRVESG
ENCSSPAPREELVPAEETEQDKEHTCETLLMCIVTVLSHGLRSGGGVGD
VLRKPSKEEPLFAARVIYDLLFFFMVIIIVLNLIFGVIIDTFADLRSEK
QKKEEILKTTCFICGLERDKFDNKTVTFEEHIKEEHNMWHYLCFIVLVK
VKDSTEYTGPESYVAEMIKERNLDWFPRMRAMSLVSSDSEGEQNELRNL
QEKLESTMKLVTNLSGQLSELKDQMTEQRKQKQRIGLLGHPPHMNVNPQ
QPA
SEQ ID NO: 16
PLALLEDWCRIMSVDEQKSLMVTGIPADFEEAEIQEVLQETLKSLGRYR
LLGKIFRKQENANAVLLELLEDTDVSAIPSEVQGKGGVWKVIFKTPNQD
TEFLERLNLFLEKEGQTVSGMFRALGQEALSPATVPCISPELLAHLLGQ
AMAHAPQPLLPMRYRKLRVFSGSAVPAPEEESFEVWLEQATEIVKEWP
SEQ ID NO: 17
MPPPAPGARLRLLAAAALAGLAVISRGLLSQSLEFNSPADNYTVCEGDN
ATLSCFIDEHVTRVAWLNRSNILYAGNDRWTSDPRVRLLINTPEEFSIL
ITEVGLGDEGLYTCSFQTRHQPYTTQVYLIVHVPARIVNISSPVTVNEG
GNVNLLCLAVGRPEPTVTWRQLRDGFTSEGEILEISDIQRGQAGEYECV
THNGVNSAPDSRRVLVTVNYPPTITDVTSARTALGRAALLRCEAMAVPP
ADFQWYKDDRLLSSGTAEGLKVQTERTRSMLLFANVSARHYGNYTCRAA
NRLGASSASMRLLRPGSLENSAPRPPGLLALLSALGWLWWRM
SEQ ID NO: 18
MKEKAMIKTAKMQGNVMELVGSNPPQRNWKGIAIALLVILVICSLIVTS
VILLTPAEDNSLSQKKKVTVEDLFSEDFKIHDPEAKWISDTEFIYREQK
GTVRLWNVETNTSTVLIEGKKIESLRAIRYEISPDREYALFSYNVEPIY
QHSYTGYYVLSKIPHGDPQSLDPPEVSNAKLQYAGWGPKGQQLIFIFEN
NIYYCAHVGKQAIRVVSTGKEGVIYNGLSDWLYEEEILKTHIAHWWSPD
GTRLAYAAINDSRVPIMELPTYTGSIYPTVKPYHYPKAGSENPSISLHV
IGLNGPTHDLEMMPPDDPRMREYYITMVKWATSTKVAVTWLNRAQNVSI
LTLCDATTGVCTKKHEDESEAWLHRQNEEPVFSKDGRKFFFIRAIPQGG
RGKFYHITVSSSQPNSSNDNIQSITSGDWDVTKILAYDEKGNKIYFLST
EDLPRRRQLYSANTVGNFNRQCLSCDLVENCTYFSASFSHSMDFFLLKC
EGPGVPMVTVHNTTDKKKMFDLETNEHVKKAINDRQMPKVEYRDIEIDD
YNLPMQILKPATFTDTTHYPLLLWDGTPGSQSVAEKFEVSWETVMVSSH
GAWKCDGRGSGFQGTKLLHEVRRRLGLLEEKDQMEAVRTMLKEQYIDRT
RVAVFGKDYGGYLSTYILPAKGENQGQTFTCGSALSPITDFKLYASAFS
ERYLGLHGLDNRAYEMTKVAHRVSALEEQQFLIIHPTADEKIHFQHTAE
LITQLIRGKANYSLQIYPDESHYFTSSSLKQHLYRSIINFFVECFRIQD
KLLTVTAKEDEEED

The present invention is further illustrated by the following non-limiting examples from which further features, embodiments, aspects and advantages of the present invention may be taken.

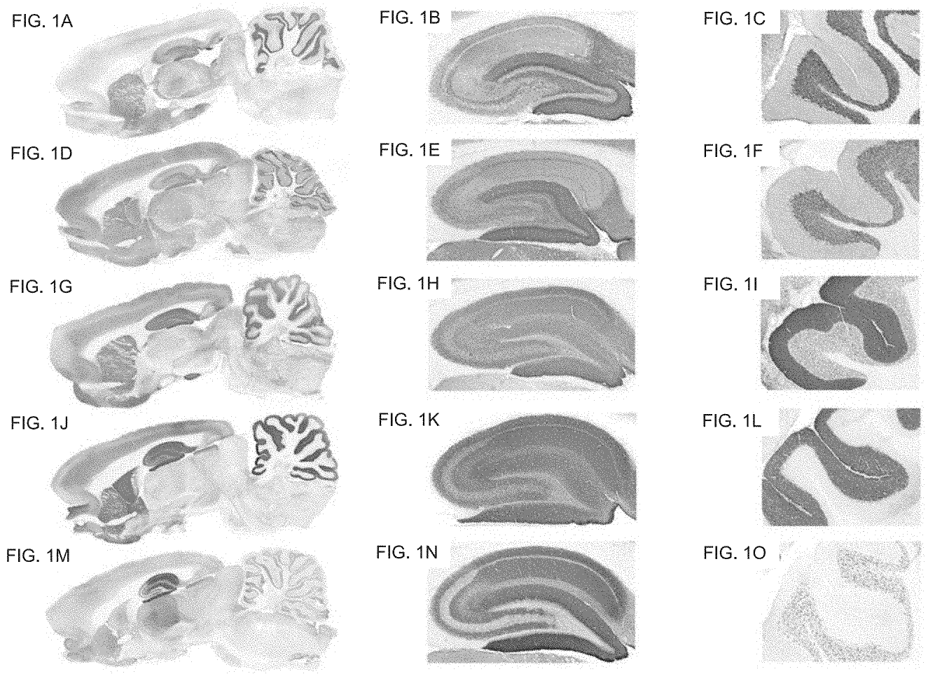

FIGS. 1A-1O shows Brain Immunostaining with CSF from patients with GluK2-abs compared with that of patients with AMPAR, NMDAR, or combined antibodies. Sagittal sections of rat brain immunostained with CSF from two patients (case 1, 1A-1C and case 2, 1D-1F) showing a novel pattern of neuropil reactivity (subsequently characterized as due to GluK2-abs). The third row of panels (1G-1I) corresponds to a patient with antibodies against AMPAR and GluK2. Both antibodies intensively react with hippocampus (1H) producing a mixed immunostaining; however, in cerebellum (1I) the staining of the molecular layer results from AMPAR antibodies, and the staining of granular cells from GluK2 antibodies (1I). For comparison with other glutamate receptor antibodies, the fourth and fifth rows correspond to CSF from a patient with anti-AMPAR (1J-1L) and a patient with NMDAR encephalitis (1M-1O) and show the distinct pattern of brain reactivity of each antibody (none of the two cases had GluK2 antibodies). Scale bars. 1G=2 mm; 1H=250 μm; 1I=250 μm.

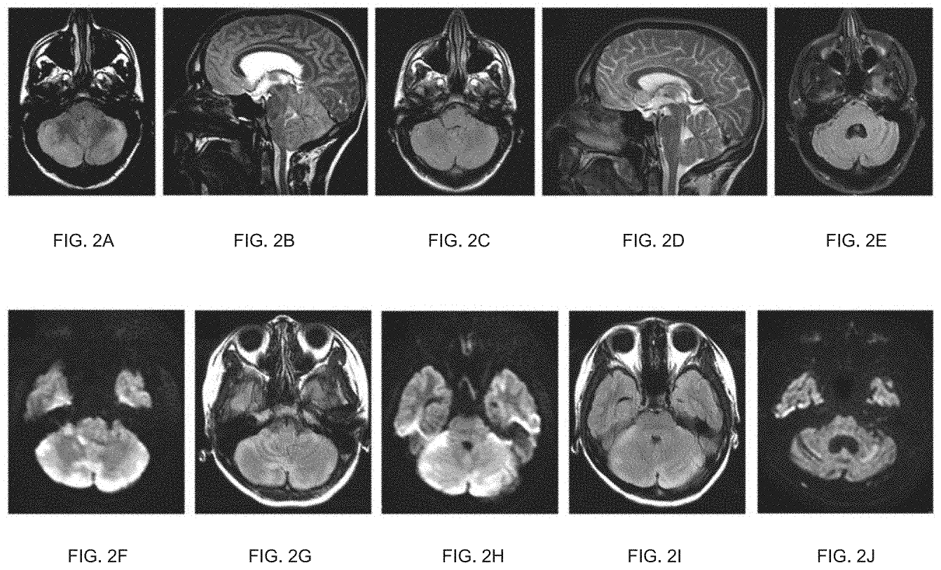

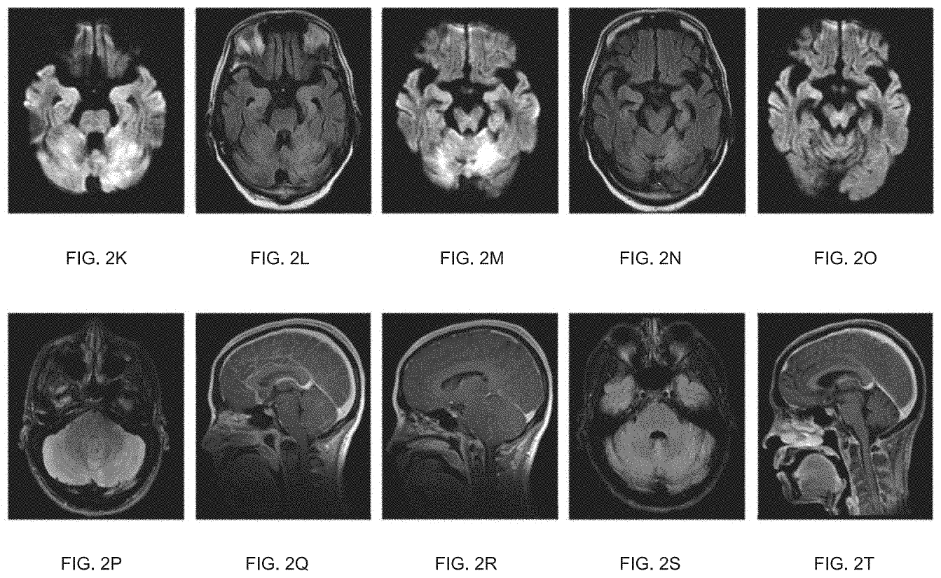

FIGS. 2A-2T: Brain MRIs in 4 patients with GluK2 antibody associated cerebellitis. 2A-2E (patient 1) show fluid-attenuated inversion recovery (FLAIR)-T2 MRI sequences of selected axial and sagittal sections obtained at symptom onset (2A,2B), two weeks later (2C,2D), and at the last follow-up (2E). Note the presence of bilateral cerebellar abnormalities with edema and compression of the 4^{th} ventricle at onset, with partial improvement two weeks later, and normal features 5 years later. 2F-2J (patient 2) show diffusion-weighted imaging (DWI) (2F,2H,2J) and FLAIR-T2 (2G,2I) MRI sequences obtained at symptom onset (2F-2I) and 2 years later (2J). There are bilateral, predominantly cortical, cerebellar abnormalities, best seen with DWI, with mass effect on the 4^{th} ventricle, and the vermis was similarly involved. No restriction was observed on apparent diffusion coefficient (ADC) maps (not shown). At the last follow-up, most of the DWI abnormalities had resolved, but there was moderate residual atrophy (2J). 2K-2O (patient 5) show DWI (2K,2M,2O) and FLAIR-T2 (2L,2N) MRI sequences obtained at symptom onset (2K-2N) and 5 weeks later (2O). Note the extensive abnormalities bilaterally involving the cerebellum and vermis, best seen in DWI sequences; there is milder involvement of the temporal lobes (left>right). On ADC maps no restriction was observed. Five weeks after symptom onset most of the DWI abnormalities had improved. 2P-2T (patient 4) show FLAIR (2P,2S) and T1 with contrast (2Q,2R,2T) MRI sequences obtained at presentation (2P-2R), and 1.5 years after disease onset (2S,2T). At disease onset, there is moderate cerebellar edema with reduction of size of the 4^{th} ventricle.

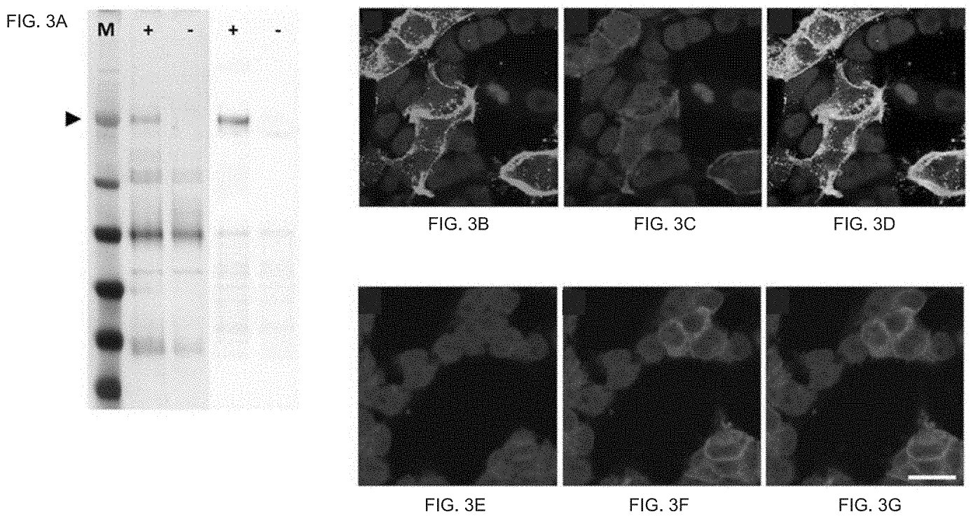

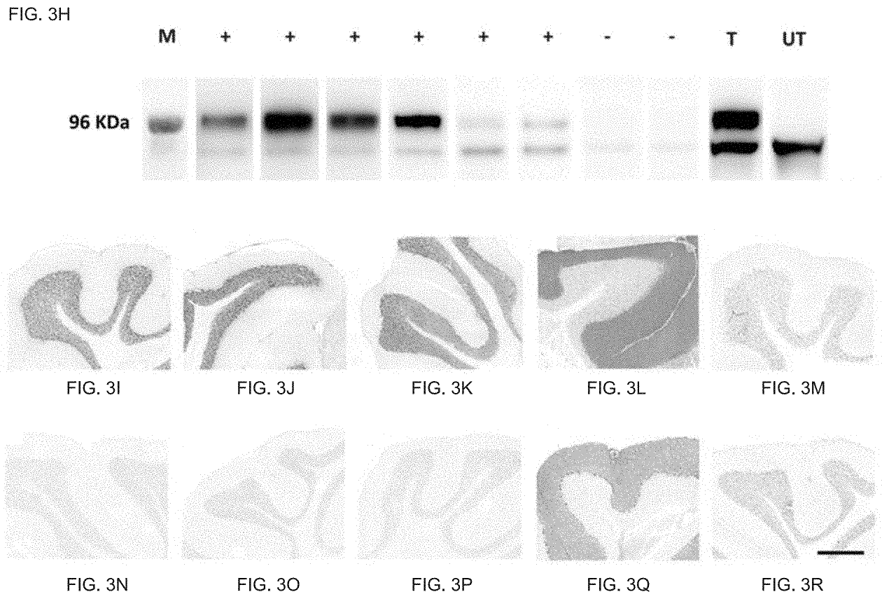

FIGS. 3A-3R: Cell-based assay and immunoprecipitation of GluK2. 3A, lanes 2 and 3 show the silver staining of proteins precipitated with serum of patient 1 (+) and a control serum (−). Molecular markers are shown in lane M (arrowhead ˜96 kDa). Lanes 4 and 5 show the corresponding immunoblot with a commercial GluK2 antibody demonstrating that the top band precipitated with patient's sample is GluK2. 3B-3D, cell-based assay with HEK293 cells expressing GluK2 immunolabelled with patient's serum antibodies (green), or a commercial antibody against Myc-tag to confirm the expression of GluK2 (red). Panel 3D shows the merged reactivities. Panels 3E-3G correspond to a similar CBA using serum from a healthy subject that demonstrates lack of reactivity with GluK2 (3E). The nuclei of the cells (blue) are shown with 4′,6-diamidino-2-phenylindole (DAPI). Scale bar=20 μm. 3H, immunoblot showing the immunoprecipitation of GluK2 from live HEK293 cells expressing GluK2 and patients' or control sera. Lanes (+) correspond to GluK2 precipitated with serum from 6 patients; lanes (−) show the lack of GluK2 precipitation using serum from 2 healthy participants; lane M is the molecular weight marker; lane T, corresponds to a lysate of HEK293 cells expressing GluK2, and lane UT correspond to a lysate of HEK293 cells not transfected with GluK2. In all lanes GluK2 was revealed with a polyclonal GluK2 antibody made in rabbit. 3I-3R, Immunostaining of cerebellum of wild-type mouse (3I-3M) and GluK2 knockout mouse (3N-3R) using CSF of 5 different patients: 3I,3N patient 1; 3J,3O patient 3; 3K,3P patient 5; 3L,3Q patient 10 (with GluK2 and AMPAR antibodies), and 3M,3R patient 14 (with GluK2 and NMDAR antibodies). Scale bar=250 μm.

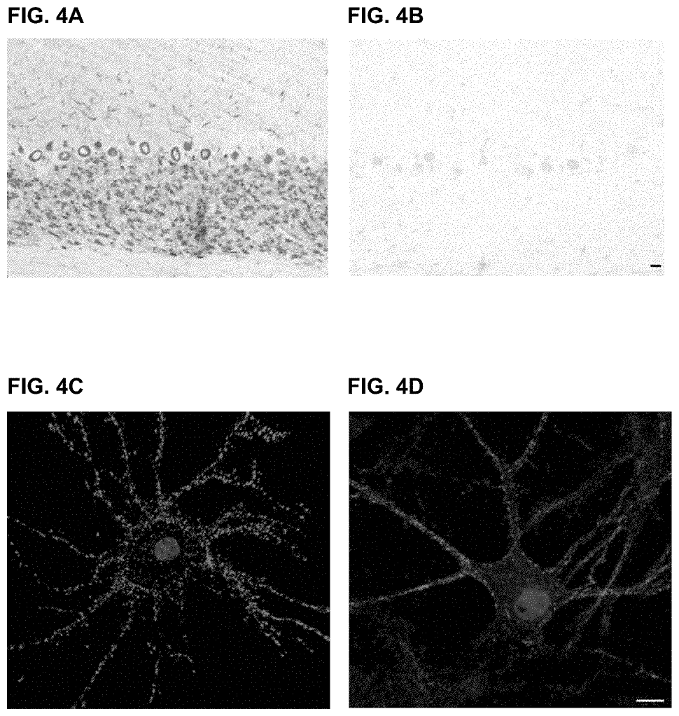

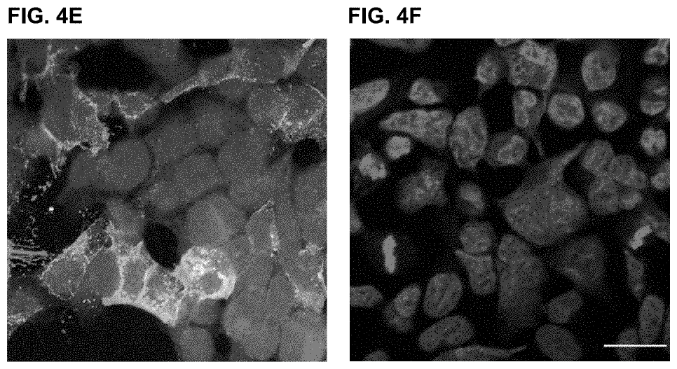

FIG. 4A-4F: Pre-absorption of patient's serum with GluK2 abrogates brain immunoreactivity. Patient's serum reactivity with cerebellum (4A,4B), live hippocampal neurons (4C,4D), and a live cell-based assay expressing GluK2 (4E,4F). Panels on the left correspond to patient's serum preabsorbed with HEK293 cells not expressing GluK2, and panels on the right correspond to the same serum preabsorbed with HEK293 cells expressing GluK2. In 4C and 4D, cells have been co-incubated with patient's serum (green fluorescence) and a commercial GluK2 antibody (red fluorescence); the yellow staining corresponds to merged reactivities. In 4E and 4F the red immunofluorescence is a commercial antibody against Myc-tag to confirm that the CBA cells express GluK2. Note that pre-absorption with GluK2 abrogates the reactivity of patient's serum with cerebellum, live neurons, and live CBA (4B,4D,4F). Scale bars 4B, 4D, 4F=20 μm.

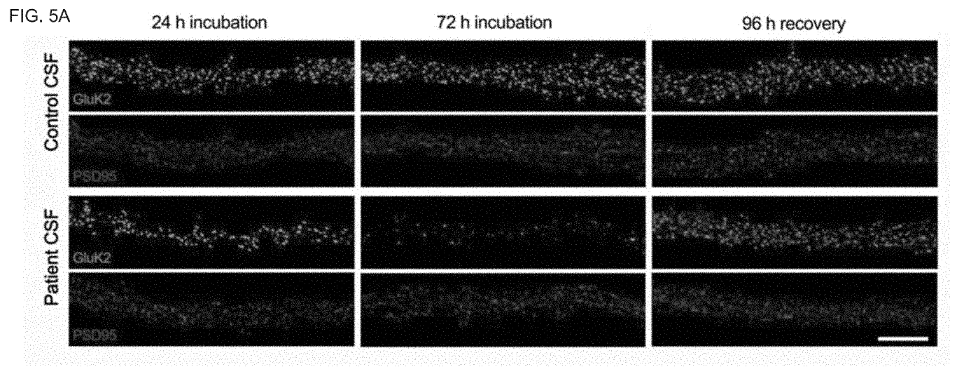

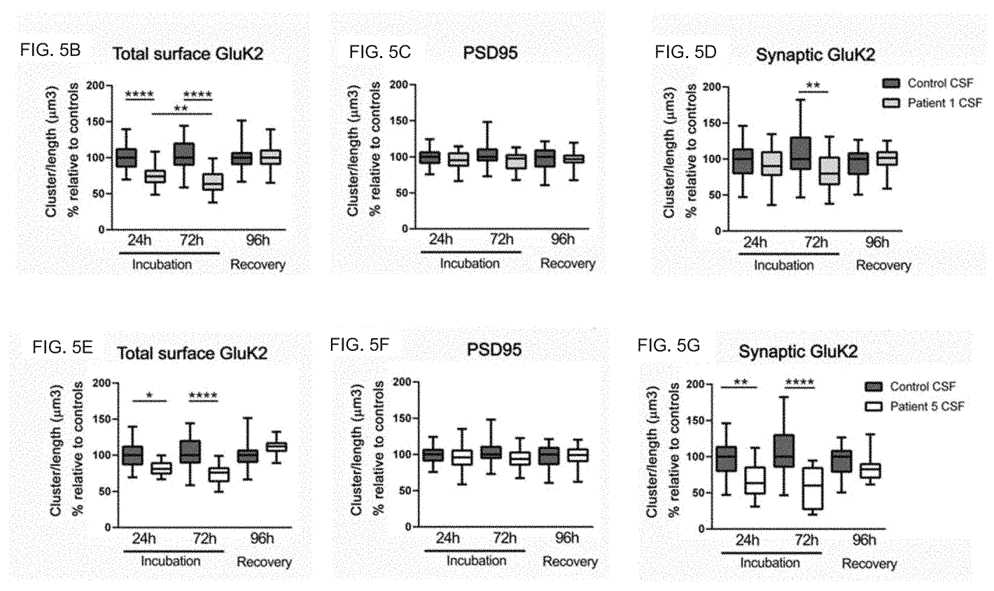

FIG. 5A-5G: Patient's antibodies cause a reduction of cell surface and synaptic GluK2 in cultured neurons. 5A shows that the CSF of a representative patient (case 1), but not control CSF, causes a progressive decrease of GluK2 clusters in representative dendrites of cultures of rat hippocampal neurons. Note that the levels of clusters return to normal after removing the antibodies from the media and allowing the neurons to recover for 96 hours. Scale bar=5 μm. 5B-5D show the quantification of these effects using CSF from patient 1 and 5E-5G from patient 5 on total neuronal surface GluK2 (5B,5E) and synaptic GluK2 (5D,5G, defined by the co-localization of surface GluK2 with PSD95). The effects on GluK2 are reversible after the 96-hour recovery. Compared with control CSF, the CSF of the patients did not change the levels of PSD95 (5C,5F), n=20 dendrites per condition, three independent experiments. Data presented as percentage against the median of the controls. Box plots show the median, and 25^{th} and 75^{th} percentiles; whiskers indicate the minimum and maximum values. Significance of treatment effect was assessed by Kruskal-Wallis with Dunn's multiple comparison *p<0.05; **p<0.01; ****p<0.0001.

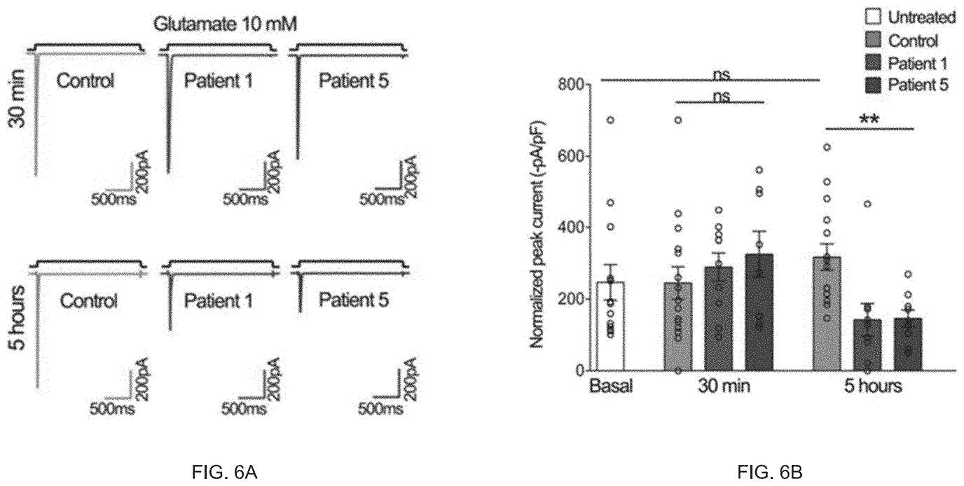

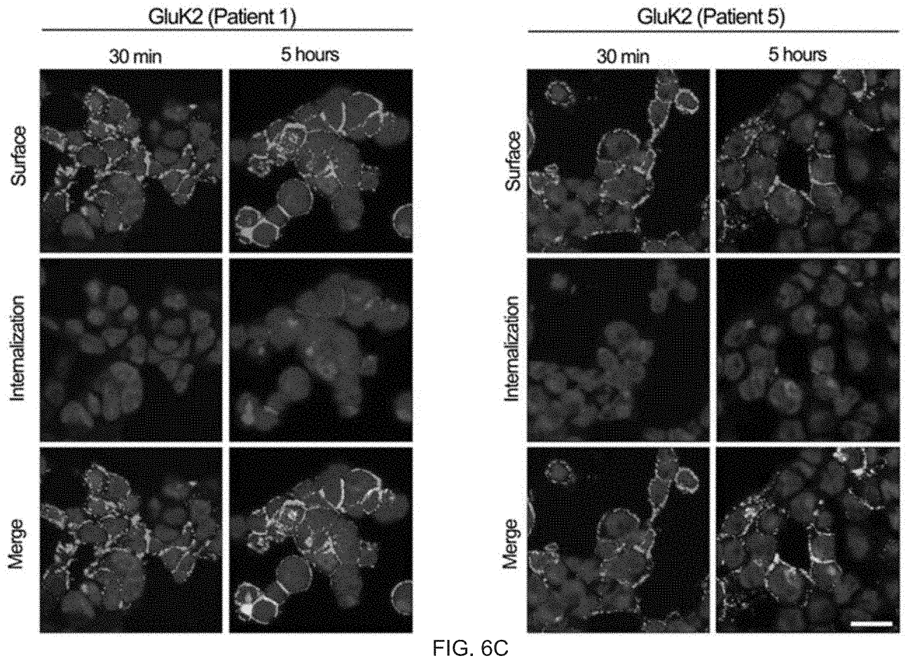

FIGS. 6A-6C: Patient's serum decreases GluK2-mediated currents: 6A,6B correspond to HEK293 cells expressing GluK2 (Q) treated for 30 minutes or 5 hours with control serum or 2 patient's serum. Current responses were activated by ultra-rapid application of 10 mM glutamate in the cells at −60 mV. In 6A the upper traces show the current responses of cells treated for 30 minutes with the indicated samples, and the lower traces show the current responses treated for 5 hours with the same samples (each of the traces represents the average of 4-7 consecutive glutamate applications). The average and S.E.M. of glutamate-evoked normalized peak currents for cells untreated (basal), incubated with control serum, or 2 patients' serum are shown in 6B. Circles denote single values for each experiment. The GluK2-mediated currents of cells treated for 30 minutes (244.7±45.39 pA/pF) or 5 hours with control serum (317±36.84 pA/pF) were similar to those of untreated cells (246.6±49.40 pA/pF) and also similar to those of cells treated for 30 minutes with two different patients' serum (Patient 1=289.1±39.22 pA/pF; Patient 2=324.9±64.1 pA/pF); p>0.99; n=15 recordings for each group from three independent experiments, Kruskal-Wallis with Dunn's comparison test. However, cells treated for 5 hours with the same two patient's serum showed a significant reduction of GluK2-mediated currents compared with the control serum (Patient 1=142.3±45.41 pA/pF, Patient 5=145.8±23.64 pA/pF vs. control=317±36.84 pA/pF); **p=0.0019 and **p=0.006 respectively; three independent experiments and at least n=8 recordings; Kruskal-Wallis with Dunn's comparison test. 6C, Binding of patients' antibodies to HEK cells expressing GluK2 after incubations of 30 minutes and 5 hours. Internalization of receptors is only visible after 5 hours (red fluorescence). Even though there is intense antibody binding at 30 minutes, no electrophysiological effects were observed (6A, 6B). These findings suggest that the reduction of GluK2-mediated currents is secondary to the antibody-mediated internalization of receptors rather than a direct antagonistic (or blocking) effect on receptor function, in which case electrophysiological changes would be visible at 30 minutes. Scale bar=10 μm.

### Examples Methods

Patients and Samples

From January 2013 until September 2020, we identified 127 patients whose serum or CSF were investigated at Hospital Clinic-IDIBAPS for suspected autoimmune CNS disorders and had antibodies that strongly reacted with the neuropil of rat brain and primary cultures of rat hippocampal or cerebellar granule neurons. Of these 127 patients, we selected serum and CSF samples from 8 patients that showed the same pattern of brain immunostaining, but different from those associated with anti-AMPAR, NMDAR, or any other known autoimmune encephalitis. As controls, we included serum of 23 healthy blood donors and serum or CSF of 596 patients with the following disorders: 73 multiple system atrophy with predominant cerebellar phenotype, 73 anti-NMDAR encephalitis, 71 anti-AMPAR encephalitis, 48 cerebellitis of unknown etiology, 40 multiple sclerosis, 37 paraneoplastic cerebellar degeneration, 30 opsoclonus-myoclonus, 29 non-hereditary degenerative ataxia, 23 neuromyelitis optica spectrum disorder, 20 encephalitis with neuronal surface antibodies other than NMDAR or AMPAR, 20 progressive supranuclear palsy, 13 Creutzfeldt-Jakob disease, and the remaining 119 cases of the initial group from which the 8 cases of the study were obtained.

The Ethic Committee of Hospital Clinic, Barcelona, approved the study (R091217-12). Patients or proxies gave consent for the storage and use of serum, CSF, and clinical information for research purposes.

Immunohistochemistry with Brain Tissue and Immunofluorescence with Cultured Neurons

Immunostaining of rat brain and wild-type or GluK2 knockout mice brain (kindly provided by Dr. Juan Lerma¹) was performed with patients serum (diluted 1:200) and CSF (1:2) using a standard immunoperoxidase technique (Ances B M, Vitaliani R, Taylor R A, et al. (2005): Treatment-responsive limbic encephalitis identified by neuropil antibodies: MRI and PET correlates, Brain, Volume 128, Issue 8). Immunofluorescence with primary cultures of rat hippocampal neurons was performed with serum (1:200) and CSF (1:5) (Lai M, Hughes E G, Peng X, et al. (2009): AMPA receptor antibodies in limbic encephalitis alter synaptic receptor location, Ann Neurol, Volume 65, Issue 4).

Immunoprecipitation

Cultures of rat cerebellar granule neurons and immunoprecipitation with patients' or control serum were performed as previously reported (Landa J, Guasp M, Petit-Pedrol M, et al. (2020): Seizure-related 6 homolog like 2 autoimmunity: Neurologic syndrome and antibody effects, Neurol Neuroimmunol Neuroinflamm). Protein precipitates were first assessed by silver staining (A44390, Thermo-Fisher Scientific, Waltham, Mass., USA), but all precipitated proteins were characterized by mass spectrometry without pre-selection of specific bands. In other experiments, HEK293 cells expressing the identified antigen (GluK2) were incubated with patients' or control serum, and subsequently lysed and precipitated as above. Precipitates from these HEK293 cells or neurons were then run in a gel, transferred to nitrocellulose, incubated with a polyclonal GluK2 antibody made in rabbit (diluted 1:500, HPA014623, Atlas antibodies, Sweden), and the reactivity developed with chemiluminescence, as reported (Landa J, et al. (2020): Seizure-related 6 homolog like 2 autoimmunity: Neurologic syndrome and antibody effects, Neurol Neuroimmunol Neuroinflamm).

Cell-Based Assays (CBA)

To determine the repertoire of patients' antibodies against each of the human subunits of kainate receptors (GluK1, GluK2, GluK3, GluK4, GluK5), HEK293 cells were transfected with plasmids containing each of the human subunit sequences tagged with MYC-DDK (all from Origene, Rockville, Md., USA) including: GRIK1 (RC222898), GRIK2 (RC222369), or GRIK3 (RC223571) which are able to form functional kainate-gated homomeric receptors. For GRIK4 (RC214488) and GRIK5 (RC224695) which only form functional receptors when co-expressed with GRIK1, GRIK2 or GRIK3, we used co-transfections with GRIK3/GRIK4 or GRIK3/GRIK5, as well as each subunit separately. The techniques of transfection and incubation of HEK293 cells with patients' samples (or CBA) are identical to those previously reported (Lai M, et al. (2009): AMPA receptor antibodies in limbic encephalitis alter synaptic receptor location, Ann Neurol, Volume 65, Issue 4. Lynch D R, Lawrence J J, Lenz S, Anegawa N J, Dichter M, Pritchett D B (1995): Pharmacological characterization of heterodimeric NMDA receptors composed of NR 1a and 2B subunits: differences with receptors formed from NR 1a and 2A, J Neurochem.). Briefly, live transfected HEK293 cells were incubated with serum (1:50) or CSF (1:5) for 1 hour at 37° C. After fixation with 4% paraformaldehyde (PFA) and permeabilization with 0.3% Triton X-100, the cells were incubated with a mouse MYC-tag antibody (diluted 1:2500, 2276S, Cell Signaling Technology, Danvers, Mass., USA) for 1 hour at room temperature (RT) followed by fluorescent secondary antibodies Alexa Fluor 488 goat anti-human (1:1000, 109-545-088, Jackson ImmunoResearch, Newmarket, UK) and Alexa Fluor 594 goat anti-mouse (1:1000, A-11005, Thermo-Fisher).

The presence of AMPAR or NMDAR antibodies was examined with CBA expressing GluA1/GluA2 subunits of AMPARs or GluN1/GluN2B subunits of NMDARs (Lai M, et al. (2009): AMPA receptor antibodies in limbic encephalitis alter synaptic receptor location, Ann Neurol, Volume 65, Issue 4. Dalmau J, Tuzun E, Wu H Y, et al. (2007): Paraneoplastic anti-N-methyl-D-aspartate receptor encephalitis associated with ovarian teratoma, Ann Neurol).

To determine whether patients' antibodies were able to internalize GluK2 in HEK293 cells, live cells were treated for 30 minutes or 5 hours with patient's serum (diluted 1:100 in the media). After washing, live cells were incubated for 1 hour with excess of Alexa Fluor 488 goat anti-human IgG (diluted 1:20; Jackson ImmunoResearch) to block all cell-surface human IgG, and then washed, permeabilized, and the internalized IgG was demonstrated with a differently labeled secondary anti-human antibody (Alexa Fluor 594 goat anti-human IgG, diluted 1:1000; 109-585-088, Jackson ImmunoResearch). All CBA studies were examined with a Zeiss AxioImager M2 fluorescent microscope with ApoTome.2 system (Carl Zeiss, Jena, Germany) and results scanned with a Zeiss LSM710 confocal microscope (Carl Zeiss).

IgG Subclass

The IgG subclass of the antibodies was assessed using CBA with HEK293 cells expressing GluK2 and secondary anti-human antibodies specific for IgG1, IgG2, IgG3 and IgG4, as reported. (Arino H, Armangue T, Petit-Pedrol M, et al. (2016): Anti-LGI1-associated cognitive impairment: Presentation and long-term outcome, Neurology)

Immunoabsorption Studies

Sera with reactivity restricted to GluK2 (i.e., absent reactivity with other GluK subunits) or sera with GluK1/2/3 reactivity were serially incubated with 8 Petri dishes (60 mm diameter) containing live HEK293 cells expressing GluK2 or mock-transfected cells. Each of the 8 sequential incubations was for 1 hour at RT. The immunoabsorbed sera were then examined with rat brain immunohistochemistry, live hippocampal neurons, and CBA, as reported (Sabater L, Gaig C, Gelpi E, et al. (2014): A novel non-rapid-eye movement and rapid-eye-movement parasomnia with sleep breathing disorder associated with antibodies to IgLON5: a case series, characterisation of the antigen, and post-mortem study, Lancet Neurol).

Quantitative Analysis of GluK2 Clusters in Cultured Neurons Using Confocal Microscopy

Rat hippocampal neurons were treated at 14 DIV with CSF of two patients (case #1 and case #5) or control CSF (both 1:20 diluted) for 24 h or 72 h. Case 1 had antibodies reacting exclusively with GluK2; in case 5 some of the GluK2 epitopes were shared with GluK1; no other antibodies were present in these two cases. After removing the antibodies by changing the media, neurons were allowed to recover for 4 days. At the indicated time points (24 h, 72 h, and post 4-day recovery), neurons that had been exposed to patient's or control CSF were washed and incubated with human IgG isolated from serum with GluK2-abs (1:200, used here as primary antibody) for 1 h at 37° C. All GluK2 cell-surface receptors labeled with human IgG were then demonstrated with Alexa Fluor 488 goat anti-human IgG (1:1000, 109-545-088, Jackson Immuno Research) for 1 hour at RT. Cells were then fixed with 4% PFA for 5 minutes, permeabilized with 0.3% Triton X-100 for 5 minutes and incubated with rabbit polyclonal PSD95 antibody (1:200, ab18258, Abcam, Cambridge, UK) for 1 hour, followed by the secondary antibody Alexa Fluor 594 goat anti-rabbit IgG (1:1000, A-11012, Thermo Fisher Scientific) for 1 hour at RT. Total and synaptic clusters of GluK2 were visualized by confocal imaging (LSM710, Carl Zeiss, Jena, Germany). Images were deconvolved using Huygens Professional version 17.04 (Scientific Volume Imaging, The Netherlands) and quantified using Imaris 8.1 software (Oxford Instruments, Abingdon UK), as reported (Planaguma J, Leypoldt F, Mannara F, et al. (2016): Human N-methyl D-aspartate receptor antibodies after memory and behaviour in mice, Brain. Planaguma J, Haselmann H, Mannara F, et al. (2016): Ephrin-B2 prevents N-methyl-D-aspartate receptor antibody effects on memory and neuroplasticity, Ann Neurol).

Electrophysiology in GluK2 Transfected HEK293 Cells

HEK293T cells were transfected with 1 μg total DNA from constructs codifying for GluK2(Q) and eGFP in a 9:1 (GluK2(Q):GFP) ratio, as reported (Lai M, et al. (2009): AMPA receptor antibodies in limbic encephalitis alter synaptic receptor location, Ann Neurol, Volume 65, Issue 4. Lynch D R, et al. (1995): Pharmacological characterization of heterodimeric NMDA receptors composed of NR 1a and 2B subunits: differences with receptors formed from NR 1a and 2A, J Neurochem). After 3-hour transfection, cells were split and yielded in glass coverslips treated with poly-L-lysine at low density. Twenty-four hours after transfection, coverslips were treated with patients' (case #1, case #5) or control serum (diluted 1/100) for 30 minutes or 5 hours, at 37° C. and 5% CO₂.

Electrophysiological recordings were obtained within less than 1 hour after serum was removed and cells were placed in the recording chamber. Untagged non-edited GluK2(Q) subunit expression was determined by patching GFP positive cells. Transfected cells were visualized with an inverted epifluorescence microscope (Axio-Vert.A1; Carl Zeiss). Cells were continuously perfused at RT with extracellular physiological solution (in mM): 145 NaCl, 2.5 KCl, 1 CaCl₂, 1 MgCl₂, 10 HEPES, and 10 glucose, adjusted to pH 7.4 with NaOH. Whole-cell recordings were made from isolated cells using electrodes with open-tip resistances of 2-4 MΩ (2.4±0.1 MΩ) made from borosilicate glass (1.5 mm o.d., 0.86 mm i.d., Harvard Apparatus, Sandbach, UK), pulled with a P-97 vertical puller (Narishige, London, UK) and, giving a final series resistance of typically 6-10 MΩ (8.7±0.4 MΩ). Glutamate 10 mM (Sigma-Aldrich) was applied to cells by piezoelectric translation (P-601.30; Physik Instrumente, Karlsruhe, Germany) of a theta-barrel application tool made from borosilicate glass (1.5 mm o.d.; Sutter Instruments) at a holding potential of −60 mV. Glutamate pulses were applied during 2 s every 20 s to allow GluK2 homomeric receptors to recover from desensitization. At the end of each recording, the adequacy of the solution exchange was tested by destroying the seal and measuring the liquid-junction current at the open pipette (10-90% rise time normally 600-800 μs). Currents were acquired at 5 kHz and low pass filtered at 2 kHz with an Axopatch 200B amplifier, Digidata 1440A interface and pClamp10 software (Molecular Devices, CA, USA). Intracellular pipette solution contained (in mM): 145 CsCl, 2.5 NaCl, 10 HEPES, 1 Cs-EGTA and 4 MgATP, adjusted to pH 7.2 with CsOH. Recordings were analysed using IGOR Pro (Wavemetrics, OR, USA) with NeuroMatic (Jason Rothman, UCL, UK).

Statistical Analyses

In confocal microscopy studies, data are presented as box plots showing the median, and 25^{th} and 75^{th} percentile; whiskers indicate the minimum and maximum values. The effect of patients' serum compared with control serum, on the number of neuronal clusters of GluK2 and PSD95 was normalized as percentage against the median of the controls and analyzed with Kruskal-Wallis With Dunn's multiple comparison test. In electrophysiological studies, data are presented in the figures as bar plots of the mean with error bars denoting the S.E.M. Comparisons between groups were performed using the non-parametric Kruskal-Wallis with Dunn's multiple comparisons test. Differences were considered significant at p<0.05. Statistical analysis was performed using GraphPad Prism version 5.0d for Mac OS X (GraphPad Software, San Diego Calif. USA, www.graphpad.com).

Results

Sera of two patients (cases 1 and 2, Table 1) were used for antigen characterization. In both cases the samples immunoreacted with an antigen expressed on the cell-surface of neurons and produced an identical pattern of brain immunostaining (FIG. 1A-F).

Patient #1:

A 24-year-old man was brought to the emergency room for acute onset headache, nausea and vomiting. Eight years earlier he was treated for Hodgkin's lymphoma and since he had no evidence of disease. Preceding the current symptoms, the patient described several weeks of dizziness and photophobia. At exam, the memory, cognition, and cranial nerve function were intact, and no motor or sensory deficits were detected. The CSF showed 52 white blood cells/mm³ and increased protein concentration (1.18 g/L). Because of the suspicion of herpes simplex encephalitis, he was started on acyclovir until the PCR results came back negative. On day 3 after admission, the headache worsened, and he developed clinical and MRI features of cerebellitis with edema and mass effect causing obstructive hydrocephalus (FIG. 2, top row). Treatment with intravenous steroids and mannitol resulted in neurologic improvement. A body fluorodeoxyglucose (FDG)-PET scan showed a hypermetabolic supraclavicular adenopathy with biopsy findings consistent with Hodgkin's lymphoma. He received chemotherapy and radiotherapy resulting in progressive improvement. At the last follow-up, 60 months after symptom onset, he was fully recovered.

Identification of GluK2 as the Main Target Antigen

Live neuron immunoprecipitation with the indicated patients' serum revealed 21 and 25 unique peptides respectively, covering the 26% and the 33% of the rat GluK2 protein sequence (NP_062182.1). Peptides from other GluK subunits were also immunoprecipitated but with lower score: GluK1 (2 unique peptides), GluK3 (2 unique peptides), GluK4 (3 unique peptides), GluK5 (5 unique peptides). The most abundant peptide was precipitated with both serum samples (peptide: MESPIDSADDLAK) and corresponds to an identical sequence shared by GluK1 (NCBI accession number: NP_001104584; 664-676 amino acids), GluK2 (NP_062182.1; 664-676 amino acids) and GluK3 (NP_001106187.1; 666-678 amino acids) located in the extracellular S2 segment of the ligand binding domain of the protein. Silver-stained gel run in parallel with an immunoblot showing the GluK2 band is shown in FIG. 3A.

GluK2 CBA Test and IgG1 Antibody Subclass

All 8 patients initially selected for their similar pattern of brain immunostaining showed robust reactivity with HEK293 cells transfected with GluK2, providing a cell-based assay (CBA) (FIG. 3B-G). Considering that the amino acid sequence of GluK2 is ˜80% identical to GluK1 and GluK3, and ˜40% identical to GluK4 and GluK5 (Bettler B, Egebjerg J, Sharma G, et al. (2020): Cloning of a putative glutamate receptor a low affinity kainate-binding subunit, Neuron), we examined the samples of all 8 patients for additional reactivity with GluK1 and GluK3, and available samples from 7 patients for GluK4 and GluK5 reactivity. In 2 cases, some of the GluK2 epitopes were shared with GluK1 or GluK3 (Table 1). In all 8 cases the GluK2-abs titers, measured by serial sample dilution, were >1:50 CSF, and >1:200 serum, and all were IgG1 (not shown).

The specificity of GluK2 antibody reactivity was confirmed by CBA (FIG. 3B-G), immunoprecipitation (FIG. 3H), and immunohistochemistry with GluK2 knockout brain tissue (FIG. 3I-R). Among the 8 index cases, samples of 7 did not react with GluK2 knockout brain including the 2 cases in which some GluK2 epitopes were shared with GluK1 or GluK3, suggesting the shared epitopes did not contribute to tissue staining. In addition, immunoabsorption with GluK2 abrogated the reactivity of the samples of the 7 patients (example shown in FIG. 4); the only case with mild residual reactivity was case 4 who had low titer additional antibodies with NMDAR, AMPAR and GlyR (not shown).

Detection of GluK2-abs in Other Anti-Glutamate Receptor Encephalitis

The finding of additional antibodies against other glutamate receptors in case 4, and the fact that GluK2 has ˜40% amino acid identity with GluA1/GluA2 subunits of the AMPAR (which are the antigens in anti-AMPAR encephalitis), and ˜25% amino acid identity with subunits of the NMDARs, led us to determine GluK2-abs in 71 randomly selected patients with anti-AMPAR encephalitis and 73 with anti-NMDAR encephalitis. Among these 144 cases, GluK2-abs were found in 5/71 with anti-AMPAR encephalitis and 1/73 with anti-NMDAR encephalitis. All 6 GluK2-abs positive patients in this group had high NMDAR and AMPAR antibody titers (all CBA, CSF titer >1:200) and relative lower titers of GluK2-abs (CSF CBA <1:50). A summary of the clinical information, antibody associations, and co-morbidities is provided in the last section of results. GluK2-abs were not found in the serum or CSF of the remaining 458 patients with autoimmune or neurodegenerative disorders or in the serum of 23 healthy blood donors.

Patients' Antibodies Cause a Decrease of GluK2 Clusters in Live Rat Hippocampal Neurons

Neurons treated with patient's CSF (cases 1 and 5) compared with those treated with control CSF, showed a significant reduction of cell-surface clusters of GluK2 (FIG. 5). Longer treatment with patient's CSF (e.g., 72 vs 24 hours) caused more robust effects (FIG. 5A,B,E).

In contrast, the clusters of PSD95 were unaffected (FIG. 5A,C,F). After changing the media to remove the antibodies, allowing the neurons to recover for 4 days, the levels of GluK2 clusters returned to values similar to those of controls (FIG. 5A,B, D,E,G).

Patients' Antibodies Impair GluK2-Mediated Currents

The effects of patients' antibodies on GluK2-mediated currents were examined with samples from patients 1 and 5 using HEK293 cells expressing GluK2 (FIG. 6A, B). Compared with non-treated cells or cells treated with serum of a healthy subject, the cells treated with patients' sera showed a significant decrease of GluK2-mediated currents (FIG. 6A, B). The impairment of GluK2-mediated currents occurred only in cells that had been exposed for several hours (˜5 hours) to patients' serum but not in cells that had very short incubations (e.g., 30 minutes). Considering that after 30 minutes incubation there was extensive antibody binding to GluK2 (FIG. 6C), the lack of electrophysiological effects by the bound antibodies suggests that they do not block or have a direct antagonistic effect on GluK2 function. Thus, the reduction of GluR2-mediated currents observed at 5 hours is likely due to receptor internalization and clustering (Crisp S J, Dixon C L, Jacobson L, et al. (2019): Glycine receptor autoantibodies disrupt inhibitory neurotransmission, Brain) This paradigm is similar to that of other glutamate receptor antibodies (NMDAR or AMPAR) which impair receptor function mainly by internalization (Haselmann H, Mannara F, Werner C, et al. (2018): Human autoantibodies against the AMPA receptor subunit GIuA2 induce receptor reorganization and memory dysfunction, Neuron. Moscato E H, Peng X, Jain A, Parsons T D, Dalmau J, Balice-Gordon R J. (2014): Acute mechanisms underlying antibody effects in anti-N-methyl-D-aspartate receptor encephalitis, Ann Neurol), and is different from that of glycine receptor antibodies which cause functional blocking of the receptor after very short (<30) incubations (Crisp S J, et al. (2019): Glycine receptor autoantibodies disrupt inhibitory neurotransmission, Brain).

Clinical and Immunological Spectrum of GluK2 Autoantibodies

A summary of the 8 patients that originated this study is shown in Table 1 (Cases #1-8). The median age at disease onset was 28 years (range: 14-75 years), 5 were male. In all but one patient (who had previous HIV-related neurological symptoms) the new onset of neurological symptoms progressed rapidly (<6 weeks) until reaching the peak of the disease. Four patients, median age 19 years (range 14-33) presented with prominent clinical manifestations compatible with cerebellitis, including opsoclonus in one of them. Two of them developed obstructive hydrocephalus, one treated with ventricular drainage and the other controlled with mannitol. Three patients (23, 67 and 75 years old) developed a more diffuse encephalitis with prodromal flu-like symptoms followed by limb or gait ataxia (2 cases) and confusion, disorientation, delusional thoughts, psychomotor agitation, myoclonus, or seizures. The other patient was a 73-year-old man with history of alcohol abuse, HIV positive for 13 years (well controlled with 4 antiretrovirals), who developed rapid (˜3 months) neurological deterioration without evidence of HIV exacerbation (CD4 count 240/ml; viral load suppressed) and CSF inflammatory findings (20 WBC/ml; 5 oligoclonal bands, normal tau and phospho-tau) that suggested an autoimmune encephalitis (Table 1, case 6).

MRI studies were available from 7 patients: 4 showed multifocal T2-FLAIR abnormalities in the cerebellum with or without cerebral involvement (FIG. 2, 1^{st} and 2^{nd} rows), one (case #5) had a clinical-radiological dissociation with mild cerebellar symptoms but extensive MRI cerebellar abnormalities (FIG. 2, 3^{rd} row). Another patient showed moderate cerebellar edema (FIG. 2, 4^{th}, row) with increased FDG-PET activity, and the remaining 2 patients had brain and cerebellar atrophy (not shown). Six of these 7 patients had CSF pleocytosis. Two patients had active tumors (1 relapsing Hodgkin lymphoma, and 1 ovarian teratoma) that were successfully treated, and 1 had a retroperitoneal teratoma removed 1 year before neurological symptom onset, without tumor relapse. Seven patients were treated with steroids, and 2 of them received immunomodulation (patient 6: IVIg; and patient 4: IVIg, plasma exchange, rituximab, cyclophosphamide). Three patients had partial or full recovery, 2 died in the acute phase of the disease (1 multiple systemic complications, 1 sepsis), 1 with HIV died 27 months after disease onset of unknown cause, and 1 was lost to follow-up. From the remaining patient (case 3) information beyond symptom presentation was not available: however, she was a 14-year-old girl who developed cerebellitis with acute cerebellar ataxia.

Separate from the 8 index cases with GluK2-only or predominant antibodies, there were 5 patients with anti-AMPAR and 1 with anti-NMDAR encephalitis that had concurrent GluK2-abs (Table 2). Four of the 5 cases with AMPAR antibodies had tumors (3 thymomas, 1 SCLC). The 3 cases with thymoma had several additional antibodies (2 CRMP5; 1 AChR, CASPR2, and GABAbR) and all showed a pattern of brain immunostaining composed of mixed reactivities (FIG. 1, 3^{rd} row). In one of the cases with concurrent AMPAR antibodies and in the case with NMDAR antibodies, the brain immunostaining did not show the GluK2 reactivity. None of the 6 patients with concurrent antibodies developed clinical-radiological features of cerebellar dysfunction, and all showed a syndrome compatible with the concurrent antibodies.

Discussion

We found that glutamate kainate receptors containing GluK2 (previously known as GluR6) are the target of antibodies in some patients with encephalitis or cerebellitis of unclear etiology, and that these antibodies are likely pathogenic. This is supported by several findings: 1) the antibodies react with extracellular GluK2 epitopes in cultures of live rat hippocampal neurons and GluK2-expressing HEK293 cells; 2) they are IgG1 and internalize GluK2 receptors leading to a significant, but reversible, decrease of synaptic and extrasynaptic clusters of GluK2; 4) they also cause a significant reduction of GluK2-mediated currents in HEK293 cells expressing these receptors, and 5) some patients responded to empiric steroids or first line immunotherapy.

The kainate receptors are tetrameric ionotropic glutamate receptors that may include GluK1, GluK2, GluK3, GluK4 or GluK5, previously known as GluR5, GluR6, GluR7, KA1 and KA2 (Contractor A, Mulle C. Swanson G T (2011): Kainate receptors coming of age: milestones of two decades of research, Trends Neurosci). GluK1, GluK2 and GluK3 form functional homo- and heterotetrameric receptors, whereas GluK4 and GluK5 form functional receptors only when co-expressed with GluK1 to GluK3 (Contractor A, et al. (2011): Kainate receptors coming of age: milestones of two decades of research, Trends Neurosci). As in the case of the GluA2 subunit of the AMPARs, GluK1 and GluK2 present Q/R editing at the second transmembrane domain (TM2). This Q-to-R substitution abolishes Ca²⁺ permeability while increases Cl⁻ permeation of the channel. (Egebjerg J, Heinemann S F (1993): Ca2+ permeability of unedited and edited versions of the kainate selective glutamate receptor GluR6, Proc. Natl. Acad. Sci. USA, Volume 90. Burnashev N, Villarroel A, Sakmann B (1996): Dimensions and ion selectivity of recombinant AMPA and kainate receptor channels and their dependence on Q/R site residues, J Physiol, Volume 496, Part 1, Page 165-173) In addition, Q residues account for larger conductance and inward rectification (Sommer B, Kohler M, Sprengel R, Seeburg P H (1991): RNA editing in brain controls a determinant of ion flow in glutamate-gated channels, Cell). In the current study Q/R editing did not modify the antibody reactivity with GluK2 (data not shown).

The kainate receptors are unconventional members of the glutamate receptor family in that, different from NMDAR or AMPAR, they are not predominantly found in excitatory postsynaptic complexes. In addition to function as ionotropic receptors, kainate receptors act as modulators for synaptic transmission and neuronal excitability interacting with metabotropic signaling pathways (Contractor A, et al. (2011): Kainate receptors coming of age: milestones of two decades of research, Trends Neurosci). They play a crucial role as presynaptic regulators of neurotransmitter release at both excitatory and inhibitory synapses by mechanisms not completely understood (Pinheiro P S, Mulle C (2006): Presynaptic glutamate receptors: physiological functions and mechanisms of action, Nat Rev Neurosci. Lerma J. Kainate (2006): receptor physiology, Curr Opin Pharmacol), are able to facilitate short-term and long-term plasticity (Contractor A, et al. (2011): Kainate receptors coming of age: milestones of two decades of research, Trends Neurosci. Schmitz D, Mellor J, Breustedt J, Nicoll R A (2003): Presynaptic kainate receptors impart an associative property to hippocampal mossy fiber long-term potentiation. Nat Neurosci) and can act as modulators of GABAergic transmission (Lourenco J, Cannich A, Carta M, Coussen F, Mulle C, Marsicano G (2010): Synaptic activation of kainate receptors gates presynaptic CB(1) signaling at GABAergic synapses, Nat Neurosci, Page 197-204).

Our initial goal to characterize the autoantigen of a group of 8 patients with antibodies that showed a similar pattern of brain immunostaining was expanded after finding that the antigen was GluK2, which has a protein sequence highly similar to other glutamate receptor subunits (˜80% identity with GluK1, GluK3; ˜40% with GluK4, GluK5 and GluA1, GluA2 of AMPAR; and ˜25% with NMDAR subunits) (Bettler B, et al. (2020): Cloning of a putative glutamate receptor: a low affinity kainate-binding subunit, Neuron). Thus, it was not surprising that the antibodies of some patients cross-reacted with epitopes shared with GluK1/GluK3, as demonstrated by immunoabsorption studies. However, investigations with patients that had otherwise classical anti-AMPAR or NMDAR receptor encephalitis resulted in the identification of a group of 6 patients, mostly with AMPAR antibodies, who had concurrent GluK2-abs. Compared with the 8 index cases with GluK-only or predominant antibodies, those with concurrent anti-AMPAR or anti-NMDAR encephalitis were more likely to have active tumors (mainly thymoma), multiple autoantibodies, and the pattern of brain immunostaining was usually composed of the expected mix of immunoreactivities. Moreover, in this group of patients the syndrome and outcome were consistent with those of the accompanying antibodies and tumors. These findings suggest that the GluK2 epitopes in both groups of patients may differ and are probably less clinically relevant in the second group. Future studies with immunocompetition between samples representative of both groups of patients may clarify this hypothesis, but the currently available small amounts of serum/CSF prevented to perform this experiment.

We found that a prolonged treatment (>5 hours) of GluK2-expressing HEK293 cells with patients' samples induced a robust reduction of GluK2-mediated currents that was not observed after a short treatment (˜30 minutes), suggesting that the impairment of GluK2-currents was secondary to the reduced cell-surface receptors (remaining fraction, not internalized) rather than an antagonistic effect of the antibody on receptor function. This paradigm is similar to the pathogenic mechanisms described in other anti-glutamate receptor encephalitis (NMDAR (Hughes E G, Peng X, Gleichman A J, et al. (2010): Cellular and synaptic mechanisms of anti-NMDA receptor encephalitis, J Neurosci. Mikasova L, P. D R, Bouchet D, et al. (2012): Disrupted surface crosstalk between NMDA and Ephrin-B2 receptors in anti-NMDA encephalitis, Brain, Page 1606-1621) or AMPAR (Haselmann H, Mannara F, Werner C, et al. (2018): Human autoantibodies against the AMPA receptor subunit GluA2 induce receptor reorganization and memory dysfunction, Neuron. Peng X, Hughes E G, Moscato E H, Parsons T D, Dalmau J, Balice-Gordon R J (2015): Cellular plasticity induced by anti-alpha-amino-3-hydroxy-5-methyl-4-isoxazolepropionic acid (AMPA) receptor encephalitis antibodies, Ann Neurol, Volume 77, Page 381-398)) that are largely mediated by internalization of receptors, whereas for GlyR antibodies a robust antagonistic effect (along with mild-moderate internalization), have been demonstrated. (Crisp S J, et al. (2019): Glycine receptor autoantibodies disrupt inhibitory neurotransmission, Brain. Peng X, Hughes E G, Moscato E H, Parsons T D, Dalmau J, Balice-Gordon R J (2015): Cellular plasticity induced by anti-alpha-amino-3-hydroxy-5-methyl-4-isoxazolepropionic acid (AMPA) receptor encephalitis antibodies, Ann Neurol, Volume 77, Page 381-398)

Although several autoimmune encephalitis can manifest with cerebellar symptoms, they almost never present as acute cerebellitis (Dalmau J, Graus F (2018): Antibody-mediated encephalitis, N Engl J Med, Volume 378, Page 840-851). The fact that 4/8 patients with GluK2-only or predominant antibodies developed early and prominent clinical or MRI findings of cerebellar-brainstem dysfunction or cerebellitis (causing obstructive hydrocephalus in two) is notable and provide a clue to suspect this disorder. In general, most reported patients with acute cerebellitis are children or young adults and they usually respond to steroids (Van Samkar A, Poulsen M N F, Bienfait H P, Van Leeuwen R B (2017): Acute cerebellitis in adults: a case report and review of the literature, BMC Res Notes. Emelifeonwu J A, Shetty J, Kaliaperumal C, et al. (2018): Acute Cerebellitis in Children: A Variable Clinical Entity, J Child Neurol, Volume 33, Page 675-684). GluK2-abs represent the first relevant antibody found in patients with this syndrome. In 5 Japanese children with acute cerebellitis, IgM or IgG antibodies against the glutamate receptor delta 2 were identified (Shimokaze T, Kato M, Yoshimura Y, Takahashi Y, Hayasaka K (2007): A case of acute cerebellitis accompanied by autoantibodies against glutamate receptor delta2, Brain Dev, Volume 29, Page 224-226. Kubota M, Takahashi Y (2008): Steroid-responsive chronic cerebellitis with positive glutamate receptor delta 2 antibody, J Child Neurol, Volume 23, Page 228-230). Unlike the GluK2-abs reported here, the clinical or pathogenic significance of glutamate receptor delta 2 antibodies is unclear as they have been also described in multiple different disorders (Berridge G, Menassa D A, Moloney T, et al. (2018): Glutamate receptor delta2 serum antibodies in pediatric opsoclonus myoclonus ataxia syndrome. Neurolog, Volume 91, Page 714-723. Fukuoka T, Takeda H, Ohe Y, Deguchi I, Takahashi Y, Tanahashi N (2012): Anti-glutamate receptor delta2 antibody-positive migrating focal encephalitis, Clin Neurol Neurosurg).

In contrast to the group of patients with GluK2-only or predominant antibodies, none of the 6 patients with GluK2-abs concurrent with anti-AMPAR encephalitis (with or without CRMP5 antibodies) or anti-NMDAR encephalitis, and none of the additional randomly selected 138 patients with anti-NMDAR or AMPAR encephalitis, developed acute cerebellitis, obstructive hydrocephalus, or opsoclonus-myoclonus (data not shown). However, some of these features, such as cerebellar dysfunction with AMPAR antibodies (Joubert B, Kerschen P, Zekeridou A, et al. (2015): Clinical spectrum of encephalitis associated with antibodies against the alpha-amino-3-hydroxy-5-methyl-4-isoxazolepropionic acid receptor. Case series and review of the literature, JAMA Neurol, Volume 72, Page 1163-1169), or opsoclonus with NMDAR antibodies, have been reported and it is unknown whether they had GluK2-abs.

Our study has limitations posed by the retrospective analysis and small number of cases that usually occurs in first descriptions of autoimmune encephalitis (Lai M, et al. (2009): AMPA receptor antibodies in limbic encephalitis alter synaptic receptor location, Ann Neurol, Volume 65, Issue 4. Dalmau J, et al. (2007): Paraneoplastic anti-N-methyl-D-aspartate receptor encephalitis associated with ovarian teratoma, Ann Neurol. Lancaster E, Lai M, Peng X, et al. (2010): Antibodies to the GABA(B) receptor in limbic encephalitis with seizures: case series and characterisation of the antigen, Lancet Neurol. Petit-Pedrol M, Armangue T, Peng X, et al. (2014): Encephalitis with refractory seizures, status epilepticus, and antibodies to the GABAA receptor: a case series, characterisation of the antigen, and analysis of the effects of antibodies, Lancet Neurol, Volume 13, Page 276-286) It is unclear the long-term outcome of patients with GluK-only antibodies because only 2 received immunomodulation (the rest of assessable cases only received steroids), 2 died in the acute phase of systemic complications, and 1 died 27 months after an episode of rapid progression of symptoms superimposed to slow progression of HIV-related deficits. Yet, 3 patients showed substantial improvement, 1 with full recovery, suggesting that prompt diagnosis and an immunotherapy approach similar to that used in other autoimmune encephalitis may be effective.

The current findings have important clinical implications. GluK2 antibody-associated encephalitis should be suspected in cases of rapid presentation of encephalitis of unknown cause with clinical-radiological cerebellar involvement, ranging from mild cerebellar symptoms to severe cerebellitis, which may be accompanied by extensive MRI T2-FLAIR abnormalities not restricted to cerebellum. Accompanying findings may include encephalopathy (memory deficit, behavioral change, seizures), signs of corticospinal tract involvement (hyperreflexia, upgoing toes, ataxic-spastic gait), or opsoclonus-myoclonus. At symptom onset, patients should be monitored for potential life-threatening posterior fossa edema and obstructive hydrocephalus. If possible, antibodies should be examined in CSF and serum with CBA and brain immunostaining. The presence of concurrent AMPA or NMDAR antibodies associates with syndromes and comorbidities usually related to these antibodies. Future studies should focus on refining epitope-syndrome associations, assess the efficacy of immunotherapy, and develop animal models of the disease.

1. A method, comprising:
detecting in a sample an autoantibody binding specifically to one or more selected from the group consisting of Gluk1, Gluk2, Gluk3, Gluk4, and Gluk5.

2. A device, comprising:
a carrier with a solid phase with one or more polypeptides each of which comprising one or more antigens selected from the group consisting of Gluk1, Gluk2, Gluk3, Gluk4, Gluk5, and a variant thereof, immobilized on the solid phase, and a) a negative control and/or b) at least one additional antigen, wherein the one or more polypeptides and the negative control or the at least one additional antigen are spatially separated on the carrier,
or
a first carrier with a solid phase comprising the one or more polypeptides, immobilized on the solid phase, and a second carrier comprising a solid phase with an immobilized a) negative control and/or b) at least one additional immobilized polypeptide comprising an antigen or a variant thereof.

3. A kit, comprising:
a polypeptide comprising one or more selected from the group consisting of Gluk1, Gluk2, Gluk3, Gluk4, Gluk5, and a variant thereof, and a carrier, wherein
a) the polypeptide is immobilized on the carrier, and the kit further comprises a means for detecting an autoantibody binding specifically to one or more selected from the group consisting of Gluk1, Gluk2, Gluk3, Gluk4, and Gluk5,
b) the polypeptide and the carrier are configured for immobilizing the polypeptide on a surface of the carrier, and the kit further comprises a means for detecting an immobilized antibody binding specifically to one or more selected from the group consisting of Gluk1, Gluk2, Gluk3, Gluk4, and Gluk5,
c) the carrier is immobilized with a means for capturing an antibody and the kit further comprises a means for detecting an immobilized antibody binding specifically to one or more selected from the group consisting of Gluk1, Gluk2, Gluk3, Gluk4, and Gluk5,
d) the carrier and a means for capturing an antibody are configured for immobilizing the means for capturing an antibody on a surface of the carrier, and the kit further comprises a means for detecting an immobilized antibody binding specifically to one or more selected from the group consisting of Gluk1, Gluk2, Gluk3, Gluk4, and Gluk5, or
e) the polypeptide is present in the form of a cell overexpressing the polypeptide, wherein the cell is the carrier.

4. The method according to claim 1, wherein detecting an antibody comprises using a carrier comprising a solid surface, whereon one or more polypeptides each comprising one or more antigens selected from the group consisting of Gluk1, Gluk2, Gluk3, Gluk4, Gluk5, and a variant thereof is immobilized.

5. The method according to claim 1, wherein the sample is from a patient having or suspected of having a neurological autoimmune disease or a cancer.

6. The method according to claim 5, wherein the neurological autoimmune disease is selected from the group consisting of PNS, cerebellar ataxia, gait ataxia, polyneuropathy, encephalitis, limbic encephalitis, epilepsy, dementia, cerebellar syndrome, and hypersensitive encephalopathy.

7. The method according to claim 5, wherein the cancer is selected from the group consisting of leukemia, graft versus host disease, and non-Hodgkin lymphoma.

8. The method according to claim 4, wherein the one or more polypeptides is a recombinant, isolated, and/or purified polypeptide.

9. The method according to claim 1, wherein the autoantibody is detected using a detection method selected from the group consisting of immunodiffusion, electrophoresis, light scattering immunoassays, agglutination, labeled immunoassays, enzyme immunoassays, chemiluminescence immunoassays, and immunofluorescence.

10. The method according to claim 1, wherein the sample is a human sample comprising a representative set of antibodies, wherein the human sample is selected from the group consisting of whole blood, plasma, serum, cerebrospinal fluid, and saliva.

11. The method according to claim 4, wherein the carrier is selected from the group consisting of a glass slide, a biochip, a microtiter plate, a lateral flow device, a test strip, a membrane, a chromatography column, and a bead.

12. The device according to claim 2, wherein the at least one additional antigen is at least one antigen selected from the group consisting of NMDAR, LgI1, AMPA1, AMPA2, CASPR2, GABA B, GABA A, DPPX, IGLON5, Hu, Yo, CV2/CRMP5, Ri, Ma2, Amphiphysin, Recoverin, RGS8, DAGLA, STX1B, AK5, AP3B2, Flotillin1+2, GRM1, GRM2, GRM5, GLURD2, ITPR1, KCNA2, NCDN, Septin 3+5+6+7+11, GLUK2, Sez6L2, and a variant thereof.

13. The device according to claim 2, wherein the carrier, the first carrier, and/or the second carrier is selected from the group consisting of a glass slide, a biochip, a microtiter plate, a lateral flow device, a test strip, a membrane, a chromatography column, and a bead.

14. The device according to claim 2, wherein the carrier, the first carrier, and/or the second carrier is a test strip, wherein one or more from the group consisting of Gluk1, Gluk2, Gluk3, Gluk4, Gluk5, and a variant thereof is immobilized in the form of a band or dot on the test strip.

15. The device according to claim 2, wherein the carrier, the first carrier, and/or the second carrier is configured for analysis using an immunofluorescence microscope.

16. The kit according to claim 3, wherein the carrier is selected from the group consisting of a glass slide, a biochip, a microtiter plate, a lateral flow device, a test strip, a membrane, a chromatography column, and a bead.

17. The kit according to claim 3, wherein the carrier is a test strip, wherein one or more from the group consisting of Gluk1, Gluk2, Gluk3, Gluk4, Gluk5, and a variant thereof is immobilized in the form of a band or dot on the test strip.

18. The kit according to claim 17, wherein one or more additional antigens are immobilized on the test strip, selected from the group consisting of NMDAR, LgI1, AMPA1, AMPA2, CASPR2, GABA B, GABA A, DPPX, IGLON5, Hu, Yo, CV2/CRMP5, Ri, Ma1, Ma2, Amphiphysin, Recoverin, RGS8, DAGLA, STX1B, AK5, AP3B2, Flotillin1+2, GRM1, GRM2, GRM5, GLURD2, ITPR1, KCNA2, NCDN, SOX1, TR(DNER), Zic4, Septin 3+5+6+7+11, Sez6L2, and a variant thereof.

19. The kit according to claim 3, wherein the carrier is configured for analysis using an immunofluorescence microscope.

20. The kit according to claim 3, wherein the variant has at least 80% sequence identity to an amino acid sequence selected from the group consisting of SEQ ID NOs 1-5.
